(19) United States
(12) Patent Application Publication
(10) Pub. No.: US 2009/0005364 A1
(43) Pub. Date: Jan. 1, 2009

(54) Azole Derivatives With Antimuscarinic Activity

(76) Inventors: Ilaria Peretto, Parma (IT); Francesca Scarpitta, Parma (IT); Elena La Porta, Parma (IT); Luca Raveglia, Parma (IT); Giuseppe Arnaldo Maria Giardina, Parma (IT); Bruno Pietro Imbimbo, Parma (IT); Andrea Rizzi, Parma (IT); Gino Villetti, Parma (IT)

Correspondence Address: LADAS & PARRY LLP, 26 WEST 61ST STREET, NEW YORK, NY, 10023, US

(21) Appl. No.: 11/794,051

(22) PCT Filed: Dec. 22, 2005
(86) PCT No.: PCT/EP05/13887

§ 371 Date: Mar. 13, 2008

(30) Foreign Application Priority Data
Dec. 23, 2004 (EP) 04030549.2

(51) Int. Cl. A61K 31/55; C07D 401/04; C07D 403/04; C07D 401/14; A61P 1/00; A61P 11/00; A61K 31/4439; A61K 31/454

(52) U.S. Cl. 514/217.09; 546/210; 514/326; 540/603; 546/274.4; 514/341

(57) ABSTRACT

The present invention relates to compounds of formula (I) wherein R1, R2, x, X, Y and B are as defined in the description for the treatment of muscarinic acetylcholine receptor mediated diseases, in particular M3 muscarinic receptor mediated diseases.

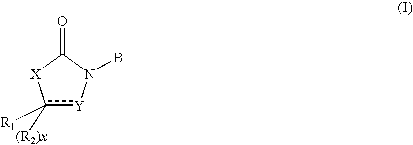

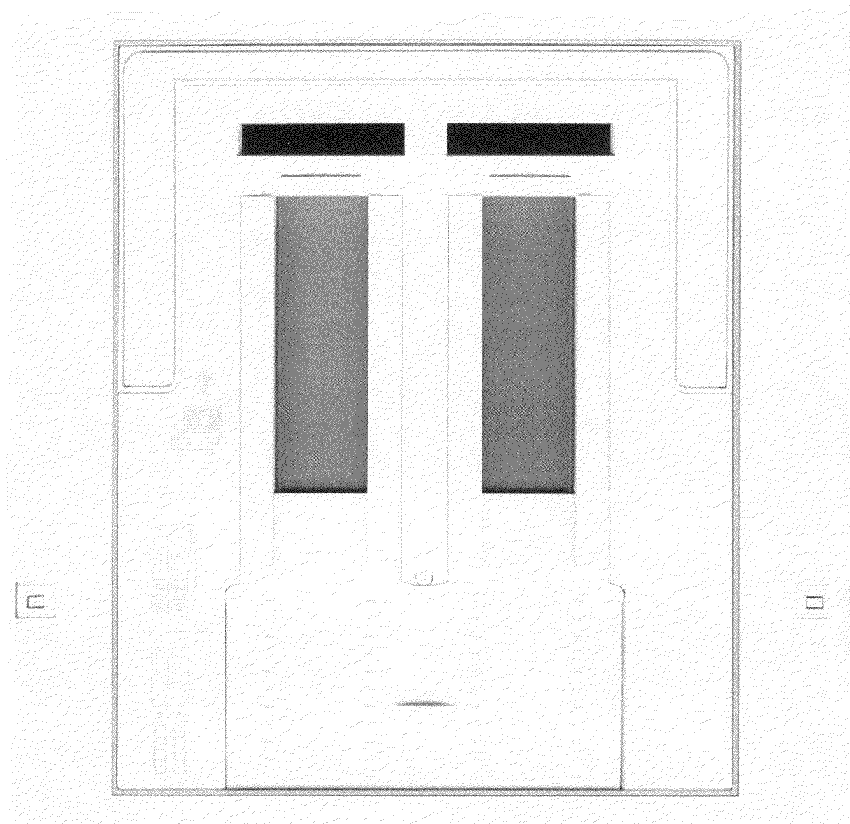

### FIELD OF THE INVENTION

The present invention relates to antimuscarinic compounds, in particular to azole derivatives.

### BACKGROUND OF THE INVENTION

The neurotransmitter acetylcholine, released from cholinergic neurons in the peripheral and central nervous systems, affects several biological processes through interaction with two major classes of acetylcholine receptors, namely the nicotinic and the muscarinic acetylcholine receptors. Muscarinic receptors are members of the G-Protein Coupled Receptors (GPCRs) superfamily and are composed of 5 receptors subtypes (M₁, M₂, M₃, M₄, M₅) that are activated by acetylcholine. These receptors are widely distributed in multiple organs and tissues and are critical to maintain the central and peripheral cholinergic neurotransmission, and can mediate both excitatory and inhibitory actions. The distribution of these receptor subtypes has been studied and documented; for example, the M₁ subtype is expressed mainly in neuronal tissues (cerebral cortex, autonomic ganglia); the M₂ subtype is located mainly in the heart (mediating cholinergically induced bradycardia), while the M3 subtype is present mainly in smooth muscle (in the airways, bladder, gastrointestinal tract) and salivary glands (Nature, 1986, 323-411; Science, 1987, 237-527). Each receptor subtype displays unique pharmacological properties (The Muscarinic Receptors, The Humana Press, Inc., 1989, Clifton, N.J.).

The therapeutic and medical aspects of muscarinic class of agonists and antagonists have been described (Molecules 2001, 6-142).

Muscarinic acetylcholine receptor disfunction has been noted in various pathophysiological states.

For example, incontinence due to bladder hypercontractility has been demonstrated to be mediated through increased stimulation of M₃ receptor subtype.

Inflammation of the gastrointestinal tract in irritable bowel syndrome (IBS) results in M₃-mediated hypermotility.

In asthma and in chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation. This disfunction results in airway hyperreactivity mediated by increased stimulation of M₃. Increased vagally-mediated reflex bronchoconstriction is seen after viral infection, exposure to ozone, or inhalation of antigen. In all cases, dysfunction of inhibitory M₂ muscarinic receptors on vagal nerve endings may contribute to an increased acetylcholine release. Because of the increased reflex bronchoconstriction resulting from these triggers of asthma attacks, anticholinergic compounds may be particularly useful for example in acute asthma. Improved anticholinergic medications, including selective M₃ antagonists, may offer effective interruption of these reflex.

Even if muscarinic agonists (pilocarpine) and antagonists (atropine) have been known for over a century, little progress has been made in the discovery of receptor subtype selective compounds and relatively few anti-muscarinic compounds are in use in the clinic, due to significant side effects. For example, although muscarinic antagonists such as atropine are potent bronchodilators, their clinical utility is limited because of the high incidence of peripheral and central adverse effects, such as tachycardia, blurred vision, dryness of mouth, constipation, etc. Subsequently, development of the quaternary derivatives of atropine, such as ipratropium (Molecules, 2001, 142-193) or other quaternary derivatives of scopine, such as tiotropium (Molecules, 2001, 142-193; Eur. Resp. J. 1993, 1031-1036), offered a way to identify more tolerated drugs. Nevertheless, most of them are not ideal anti-cholinergic bronchodilators, due to lack of selectivity for muscarinic receptor sybtypes.

U.S. Pat. No. 2,954,381 discloses 3-substituted oxazolidinediones with antiinflammatory and bronchodilatory activity;

WO 99/32481 discloses azole derivatives having muscarinic activity;

WO 01/44200 discloses azole derivatives as selective neurokinin antagonists;

WO 03/035638 discloses 4-imidazolin-2-one derivatives as MAP kinase inhibitors useful as medicaments, in particular as antiinflammatory agents;

WO 04/032856 discloses oxazolidin-2-ones as inhibitors of the chemokine receptor CCR8 useful for the treatment of respiratory diseases, such as asthma.

WO 05/072308 discloses diarepanone derivatives ad CGRP receptor antagonists useful in headache, micraine and cluster headache.

However, there is still therefore the need for highly selective muscarinic antagonists which can interact with distinct subtypes, thus avoiding the occurrence of adverse effects.

### DETAILED DESCRIPTION OF THE INVENTION

The present invention relates to compounds of formula (I) or pharmaceutically acceptable salts thereof for the treatment of muscarinic acetylcholine receptor mediated diseases, in particular M3 receptor mediated diseases.

The present invention relates to compounds of general formula (I)

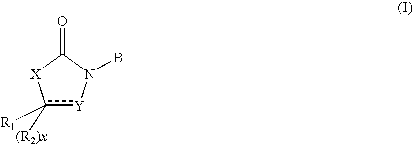

wherein:

R₁ represents

linear or branched C₁-C₇ alkyl;

C₃-C₇ cycloalkyl;

phenyl, benzyl, phenyloxymethyl, or a single or fused heterocycle, optionally substituted with one or more of the following groups: F, Cl, Br, linear or branched C₁-C₆ alkyl, C₃-C₆ cycloalkyl, methylendioxy, ethylendioxy, vinyl, CF₃, NO₂, CN, COOH, OCF₃, CH₂OR₄, OR₄, NR₄R₅, SO₂NR₄R₅, CONR₄R₅, SR₄, SO₂R₄, COR₄, wherein R₄ is H, linear or branched C₁-C₆ alkyl, phenyl, benzyl or a single or fused heterocycle optionally substituted with F, Cl, Br, linear or branched C₁-C₆ alkyl, C₃-C₆ cycloalkyl, methylendioxy, ethylendioxy, vinyl, CF₃, NO₂, CN, CH₂OH and R₅ is H, linear or branched C₁-C₇ alkyl, CO-(linear or branched C₁-C₇ alkyl) or R₄ and R₅ can form a single or fused heterocycle comprising up to 8 atoms;

x is 0 when [symbol] is a double bond and 1 when [symbol] is a single bond;

R₂ is H or has the same meanings as R₁
Y represents:

C═O;

CHOH;

(CH₂)ₘ, wherein m is an integer from 1 to 3;

or a CH group;

X represents:

sulfur

or a NR₇ group, wherein R₇ is hydrogen or a G-R₆ group, in which G is selected from CO, SO₂, (CH₂)ₙ, (CH₂)ₙCONH with n=0-3 and R₆ is H, a COOH group or has the same meanings as R₁;

B is selected from one of the following groups:

a1)

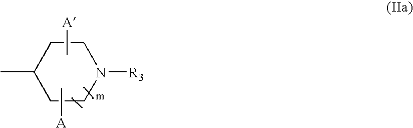

wherein A and A′ represent, independently from one another, hydrogen, linear or branched C₁-C₄ alkyl groups, with m=0-2 and R₃ is a M-R₆ group, wherein M is selected from CO, CONH, SO₂, (CH₂)ₙ, (CH₂)ₙCONH with n=1-3 and R₆ is H, a COOH group or has the same meanings as R₁;

a2)

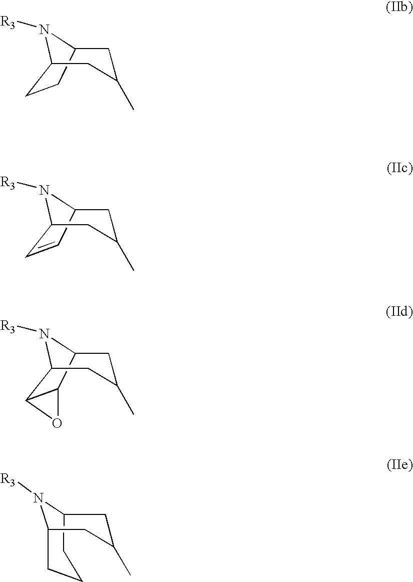

wherein A and A′ represent, independently from one another, hydrogen, linear or branched C₁-C₄ alkyl groups, with m=0-2 and R₃ is a M-R₆ group, wherein M is selected from CO, CONH, SO₂, (CH₂)ₙ, (CH₂)ₙCONH with n=0-3 and R₆ is H, a COOH group or has the same meanings as R₁;

b)

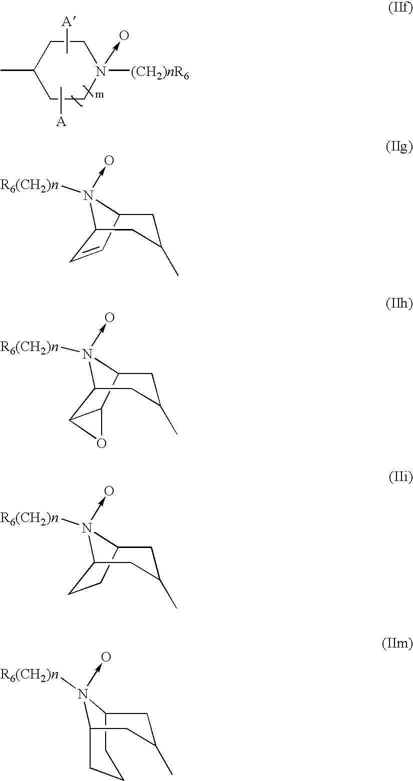

wherein R₆, m and n are as defined above;

c)

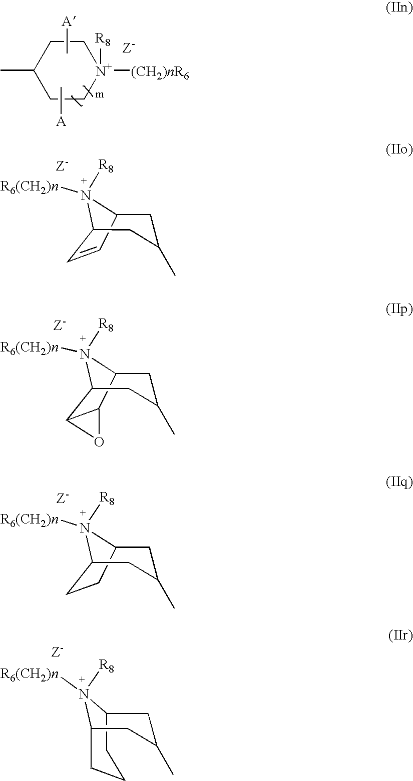

wherein:

R₆, m and n are as defined above and R₈ has the same meanings as R₁, or (CH₂)ₙR₆ and R₈, together with the nitrogen atom they are bound to, form a 4 to 7-membered heterocyclic ring, optionally substituted by a phenyl ring or optionally fused with a benzene ring or a single or fused heterocycle; and

Z- is a pharmaceutically acceptable anion, preferably selected from chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate, and more preferably selected from chloride, bromide, formate, trifluoroacetate or methanesulfonate.

The term “single or fused heterocycle” means heterocyclic rings containing from 5 to 10 ring atoms, and comprising up to 4 heteroatoms selected from S, N, O in each ring, selected from:

pyrrole, pyrazole, furan, thiophene, indole, benzofuran, benzothiophene, imidazole, oxazole, isoxazole, thiazole, benzimidazole, benzoxazole, benzothiazole, pyridine, pyrimidine, pyrazine, quinoline, isoquinoline, quinazoline, and all the corresponding saturated and partially saturated heterocycles;

More preferably, the heterocyclic ring is selected from thiophene, benzothiophene, furan, pyridine.

A first preferred group of compounds of formula (I) is the group of compounds of formula (IA)

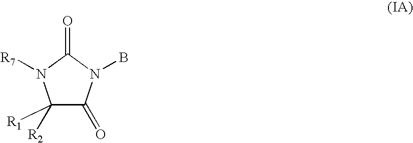

wherein:

B is a group of formula (IIa)

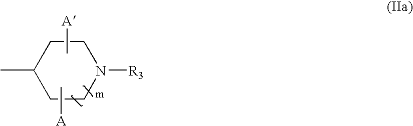

in which A and A′ are preferably hydrogen atoms, with m=0-2 and R₃ is preferably a M-R₆ group wherein M is (CH₂)ₙ with n=1 and R₆ is hydrogen, substituted or unsubstituted phenyl or single or fused heterocycle; or

a group of formula (IIb)

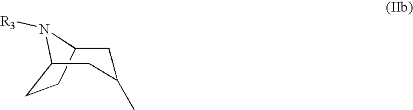

wherein R₃ is preferably a M-R₆ group wherein M is (CH₂)ₙ with n=1 and R₆ is hydrogen, methyl, substituted or unsubstituted phenyl or single or fused heterocycle;

or a group of formula (IId)

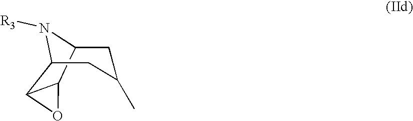

wherein R₃ is preferably a M-R₆ group wherein M is (CH₂)ₙ with n=1 and R₆ is hydrogen, methyl, substituted or unsubstituted phenyl or single or fused heterocycle;

or a group of formula (IIn)

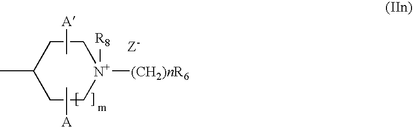

or a group of formula (IIq)

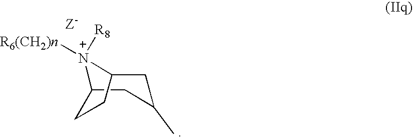

In the groups of formula (IIn) and (IIq) A and A′ are preferably hydrogen, m is 0-1, n is 1, R₆ is hydrogen, methyl, substituted or unsubstituted phenyl or single or fused heterocycle, and R₈ is selected from: methyl, 2-thienyl-propyl, cyclohexylmethyl, 2-N,N-dimethylaminoethyl, 2-tetrahydrofurylmethyl, carboxymethyl, 2-(5-aminopiridinylmethyl), 2-(SO₃H)-ethyl or (CH₂)ₙR₆ and R₈, together with the nitrogen atom they are bound to, form one of the following groups:

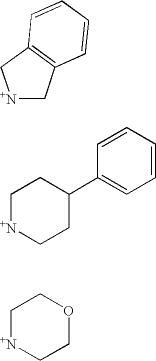

Moreover, in the compounds of formula (IA):

R₁ is preferably selected from phenyl, optionally substituted as defined above; cyclopentyl; cyclohexyl; benzyl; 2-thienyl and hydrogen;

R₂ is preferably selected from the group consisting of hydrogen, phenyl, optionally substituted as defined above, phenoxymethyl, optionally substituted as defined above; cyclohexyl; 2-thienyl and methyl and

R₇ is preferably hydrogen or a G-R₆ group wherein G is (CH₂)ₙ with n=1 and R₆ is methyl, substituted or unsubstituted phenyl or single or fused heterocycle.

A second preferred group of compounds of formula (I) is the group of compounds of formula (IB)

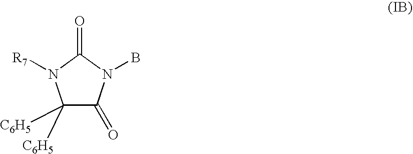

wherein:

R₇ is hydrogen;

B is a group of formula (IIa)

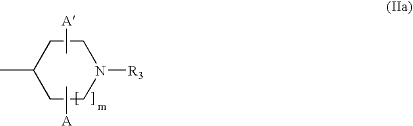

in which A and A′ are preferably hydrogen atoms, with m=0-1 and R₃ is preferably a M-R₆ group wherein M is (CH₂)ₙ with n=1 and R₆ is hydrogen or substituted phenyl or single or fused heterocycle;

or a group of formula (IIn)

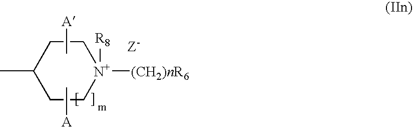

or a group of formula (IIq)

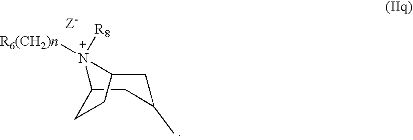

In the groups of formula (IIn) and (IIq) A and A′ are preferably hydrogen, m is 0-1, n is 1, R₆ is hydrogen, methyl, substituted or unsubstituted phenyl or single or fused heterocycle, and R₈ is selected from: methyl, 2-thienyl-propyl, cyclohexylmethyl, 2-N,N-dimethylaminoethyl, 2-tetrahydrofurylmethyl, carboxymethyl, 2-(5-aminopiridinylmethyl), 2-(SO₃H)-ethyl or (CH₂)ₙR₆ and R₈, together with the nitrogen atom they are bound to, form one of the following groups:

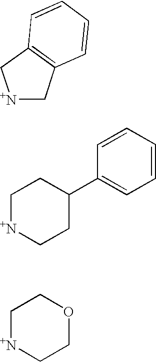

A third preferred group of compounds of formula (I) is the group of compounds of formula (IC)

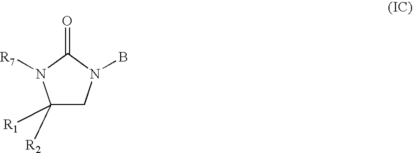

wherein, B is a group of formula (IIa)

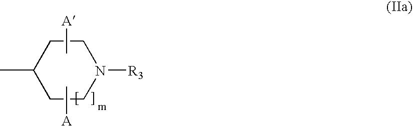

or a group of formula (IIf)

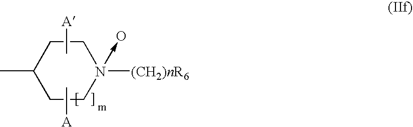

wherein A and A′ are preferably hydrogen with m=0-1 and R₃ is a M-R₆ group, wherein M is (CH₂)ₙ with n=1-3 and R₆ is hydrogen, substituted or unsubstituted phenyl, phenoxy, cyclohexyl or single or fused heterocycle,

or a group of formula (IIb)

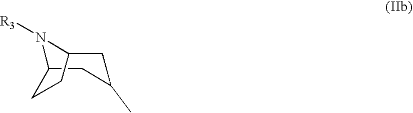

wherein R₃ is preferably a M-R₆ group, wherein M is (CH₂)ₙ with n=0 or 1 and R₆ is hydrogen, substituted or unsubstituted phenyl or single or fused heterocycle;

or a group of formula (IId)

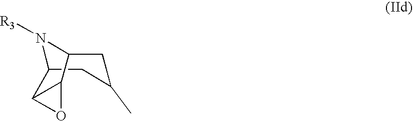

wherein R₃ is preferably a M-R₆ group wherein M is (CH₂)ₙ with n=1 and R₆ is hydrogen, methyl or substituted phenyl or fused heterocycle; or a group of formula (IIn)

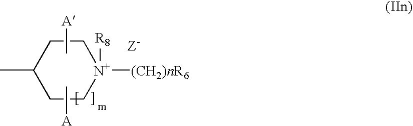

or a group of formula (IIq)

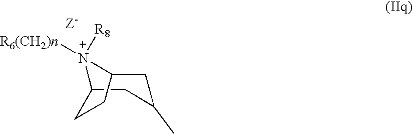

or a group of formula (IIp)

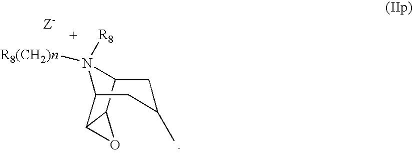

In the groups of formula (IIn), (IIq) and (IIp) A and A′ are preferably hydrogen, m is 0-2, n is 1-3, R₆ is hydrogen, phenyl, single or fused heterocycle, or C₁-C₄ alkyl optionally substituted by SR₄, SO₂R₄, CN, OR₄, COR₄, CONHR₄, wherein R₄ is selected from optionally substituted phenyl, benzyl, 2- or -3-thienyl, 2-, 3-, or 4-pyridinyl, C₁-C₄ alkyl and R₈ is selected from: methyl, 2-thienyl-propyl, cyclohexylmethyl, optionally substituted benzyl, phenoxyethyl, 2-N,N-dimethylaminoethyl; 2-tetrahydrofurylmethyl, carboxymethyl, 2-(5-aminopiridinylmethyl), 2-(SO₃H)-ethyl or (CH₂)ₙR₆ and R₈, together with the nitrogen atom they are bound to, form one of the following groups:

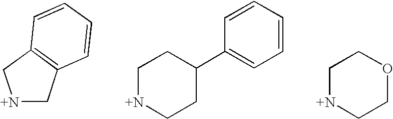

In the compounds of formula (IC):

R₁ is preferably selected from phenyl, optionally substituted as defined above, cyclopentyl, cyclohexyl, benzyl, 2-thienyl;

R₂ is preferably selected from the group consisting of hydrogen, optionally substituted phenyl or phenoxymethyl, as defined above; cyclopentyl; cyclohexyl; 2-thienyl and methyl and

R₇ is preferably hydrogen or a G-R₆ group wherein G is (CH₂)ₙ with n=1 and R₆ is hydrogen, methyl, substituted or unsubstituted phenyl or single or fused heterocycle.

A fourth preferred group of compounds of formula (I) is the group of compounds of formula (IE)

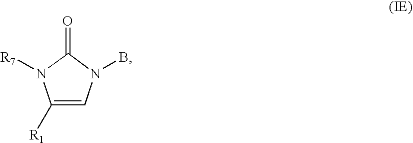

wherein:

R₁ is phenyl,

R₇ is a G-R₆ group wherein G is (CH₂)ₙ with n=1 and R₆ is phenyl and

B is preferably a group of formula (IIa)

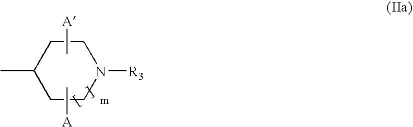

or a group of formula (IIb)

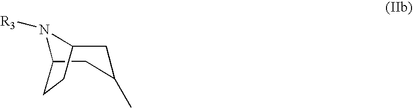

wherein R₃ is as defined above, preferably a M-R₆ group, wherein M is (CH₂)ₙ with n=0-1 and R₆ is hydrogen, substituted or unsubstituted phenyl or single or fused heterocycle.

The compounds of the invention can be prepared according to the synthetic pathway described in the following Schemes 1-9. In Schemes 3-7 and 9 the synthetic pathway has been exemplified with piperidine derivatives (corresponding to formula (IIa), in which m=1). The same synthetic pathway can be used for the preparation of the compounds of the invention bearing (IIa) (in which m=0-2), IIb, IIc, IId, IIe.

When X═NR7 and Y═CO, intermediate hydantoin derivatives (1) and (2) can be prepared according to methods described in the literature (Page, P. et al., Tetrahedron 1992, 48, 7265-7274; Stalker, R. A. et al., Tetrahedron 2002, 58, 4863-4839); representative synthetic pathways employed for the synthesis are reported in Scheme 1. Hydantoin derivatives (1), where X═NH, were prepared starting from the corresponding ketones (3a) via Bucherer-Bergs reaction with potassium cyanide and ammonium carbonate at high temperatures in a stainless steel sealed tube, or alternatively from the corresponding diketo derivatives (3b) with urea and potassium hydroxide in ethanol.

In a second procedure, the same derivatives (1) were prepared from the corresponding amino acid primary amides (4) by cyclization with urea in the same conditions described for the Bucherer-Bergs reaction (Davies, M. A. et al., J. Med. Chem. 1964, 7, 439-445). Such aminoacids can be prepared as described in the literature: for example, starting from ketoacid derivatives (5) by reaction with a Grignard reagent to introduce the R2 substituent; the hydroxyl ester thus obtained can be converted to amino amides (4) by heating with ammonia in a sealed tube (Turner, W. B. et al., J. Chem. Soc Perkin Trans. 1967, 2225-2228).

In order to prepare substituted hydantoin derivatives (2), where R7≠H, amino-amide derivatives (4) are reacted with a suitable reagent R7-Z, where Z is a suitable leaving group, in order to functionalize the amino group as in derivatives (6) (for a general review of the reactivity of amino groups, see Smith, M. B., March, J. Advanced Organic Chemistry, Wiley, 2001). Then cyclization to hydantoin is obtained as described for compounds (I) with urea in a sealed tube or alternatively in two steps by formation of the corresponding p-nitrophenyl carbamate and cyclization with sodium hydroxide (De Lucca, G. V. et al., J. Med. Chem. 2002, 45, 3794-3804).

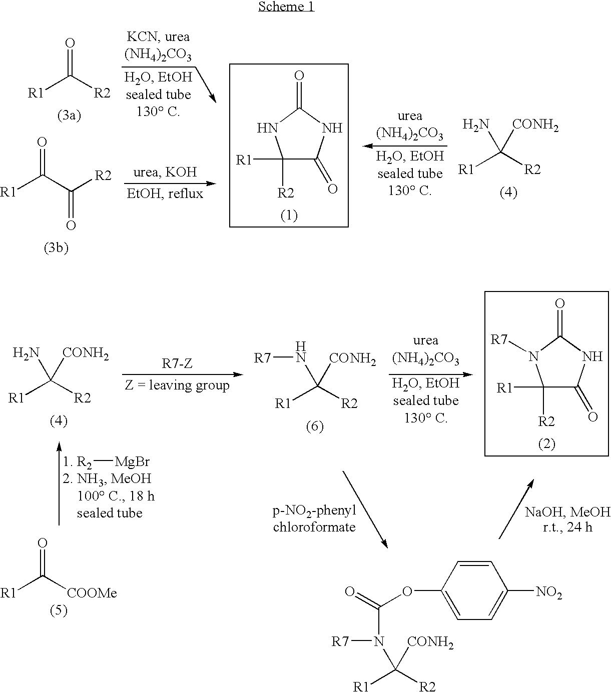

In order to prepare heterocyclic intermediates where Y═O and X═O, S, such as (7) or (8), the synthetic pathway indicated in Scheme 2 was followed.

Keto-ester or thio-ketoester derivatives (9) were reacted with Grignard compounds to give the corresponding α-hydroxy or α-mercapto esters as described in the literature (Mayrargue, J. et al., Bull. Soc. Chim. Fr. 1984, 129-132). The ester were converted to primary amides (10) by treatment with ammonia in methanol at 60° C. Cyclization was afforded by heating with urea in a sealed tube or alternatively in a two-step procedure involving the formation of the p-nitrophenyl carbonate (or thiocarbonate) and subsequent cyclization with sodium hydroxide.

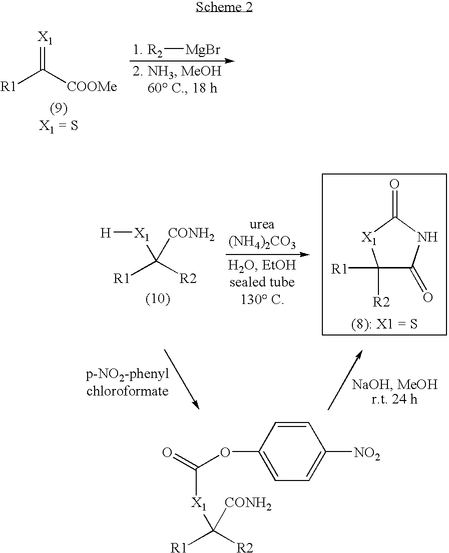

Intermediates (1), (2), (8), in which X═S, NR7, were further functionalized at the nitrogen atom in position 3 to give intermediates (11) as described in Scheme 3. Reactant (12), consisting of an amino-alcohol suitably protected at the amino group with a protecting group (PG), for example as tert-butyloxycarbonyl (BOC) or benzyloxycarbonyl (Cbz) derivative, or alternatively as benzyl or methyl derivative, was reacted with intermediates (1), (2), (8) via Mitsunobu reaction to give (11), as described in the literature (Pelletier, J. C. et al., Tetr Lett. 2001, 41, 797-800) for hydantoins or similar compounds.

In a different synthetic pathway, functionalization of intermediates (1), (2), (8), can be achieved by deprotonation of the nitrogen at position 3 and subsequent reaction with mesylate derivative (13), or with a similar derivative in which the alcohol group has been activated as leaving group. Intermediates (13) can be obtained as described in the literature (Bentley, J. et al., J. Chem. Soc. Perkin Trans. 1994, 2, 2531) from compounds (12) for example by reaction with mesyl chloride and triethyl amine in methylene chloride.

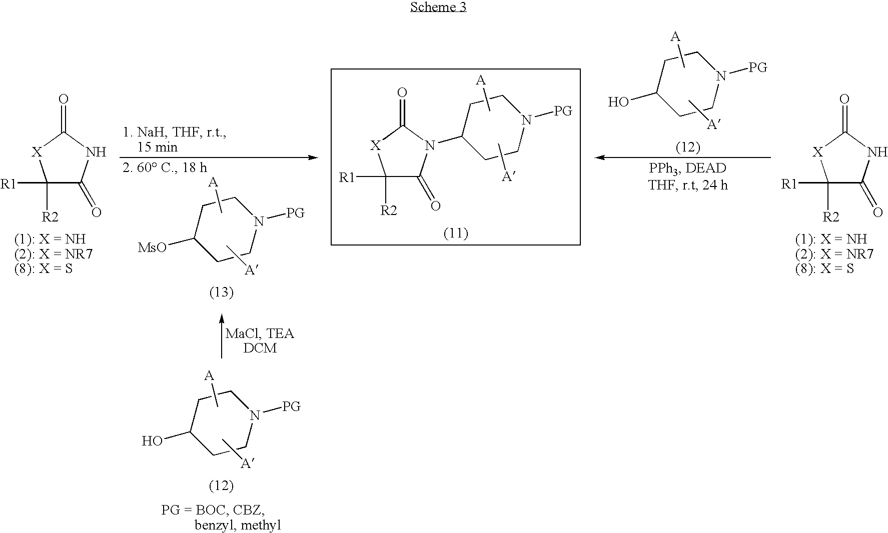

Subsequent modification of intermediates (11) were achieved as described in Scheme 4, in order to obtain derivatives in which Y═CHOH or Y═CH and R2 forms a bond with Y.

By reduction with sodium bis-(2-methoxyethoxy)aluminium hydride (Red-Al) or with a mixture of LiAlH₄ and AlCl₃ at room temperature (Knabe, J. et al., Arch. Pharm. (Weinheim), 1993, 326, 331-334), the hydroxy derivatives (14) were obtained which, if R2=H, can be transformed in the unsaturated compound (15) in mild acidic conditions. Reduction of derivatives (11) with Red-Al or with LiAlH₄+AlCl₃ at reflux in THF afforded the saturated intermediates (16) (Marquez, V. E. et al., J. Org. Chem. 1972, 37(16), 2558-2561).

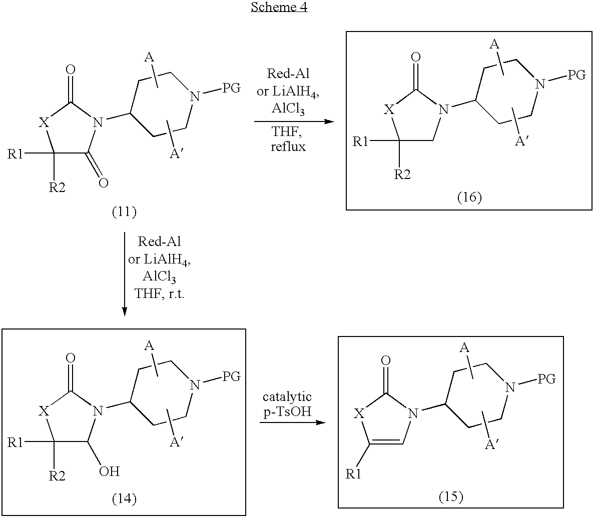

Further functionalization to introduce substituent R3 and give final compounds (17), (18), (19), (20), (21) was achieved as indicated in Scheme 5. The protecting group PG, suitably BOC or CBz or benzyl or methyl, was removed by methods described in the literature, i.e for example HCl in Et₂O for removal of BOC group (Stahl, G. L. et al., J. Org. Chem. 1978, 43, 2285), hydrogenation with palladium supported on charcoal for removal of CBz or benzyl groups (Bergman, M. et al., Chem. Ber. 1932, 65, 1192), treatment with α-chloroethyl chloroformiate and subsequent hydrolysis with sodium hydroxide for methyl group (Olofsen, R. A. et al., J. Org. Chem. 1984, 49, 2081-2082).

Compounds described as (17) can be considered final compounds, or as intermediates for the introduction of residue R3.

Residue R3 can be introduced by all means described in the literature to functionalize secondary amino groups, i.e. alkylation, reductive amination, arylation, acylation, sulphonylation, reaction with isocyanides (for a general review of the reactivity of amino groups, see Smith, M. B., March, J. Advanced Organic Chemistry, Wiley, 2001).

Compounds (18), when R3 is such that the nitrogen atom maintains the character of a tertiary amino group, can be further functionalized to give ammonium salts of type (19), by treatment with an organic or inorganic acid (for example, hydrochloric acid, hydrobromic acid, oxalic acid, fumaric acid, tartaric acid, citric acid, etc.); compounds (18) can also be transformed in quaternary ammonium salts such as compounds (20) (Lim, L. et al., Tetr. Lett. 1997, 38, 3243), or N-oxide derivatives (21) (Albin, A. et al., Heterocyclic N-Oxides, CRC Press, 1991, 31).

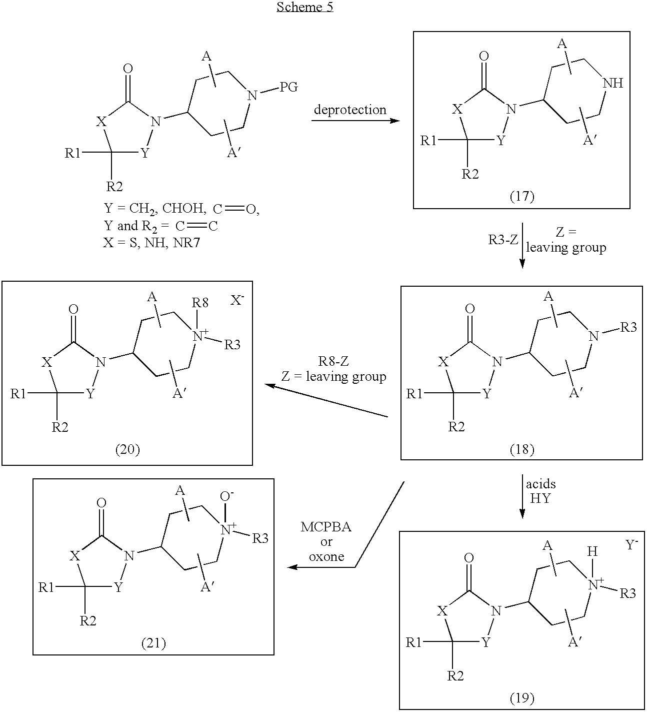

In an alternative synthetic pathway, substituent R3 can be introduced at an earlier stage of the synthesis as indicated in Scheme 6.

Compounds (22) can be functionalized by all means described in the literature to functionalize secondary amino groups, i.e. alkylation, reductive amination, arylation, acylation, sulphonylation, reaction with isocyanate, (for a general review of the reactivity of amino groups, see Smith, M. B., March, J. Advanced Organic Chemistry, Wiley, 2001) to give intermediates (23). According to the same procedure described in the previous Scheme 3, compounds (23) can be reacted with intermediates (1), (2) or (8) via Mitsunobu reaction or alkylation to give compounds (24). Such compounds can be final compounds or can be partially or totally reduced to give final compounds (26) or (27), respectively, similarly to the procedure described in the previous Scheme 4.

Moreover, all the final compounds (24), (26), (27) can be further functionalized as ammonium salts, quaternary ammonium salts or N-oxide derivatives as described in the previous Scheme 5.

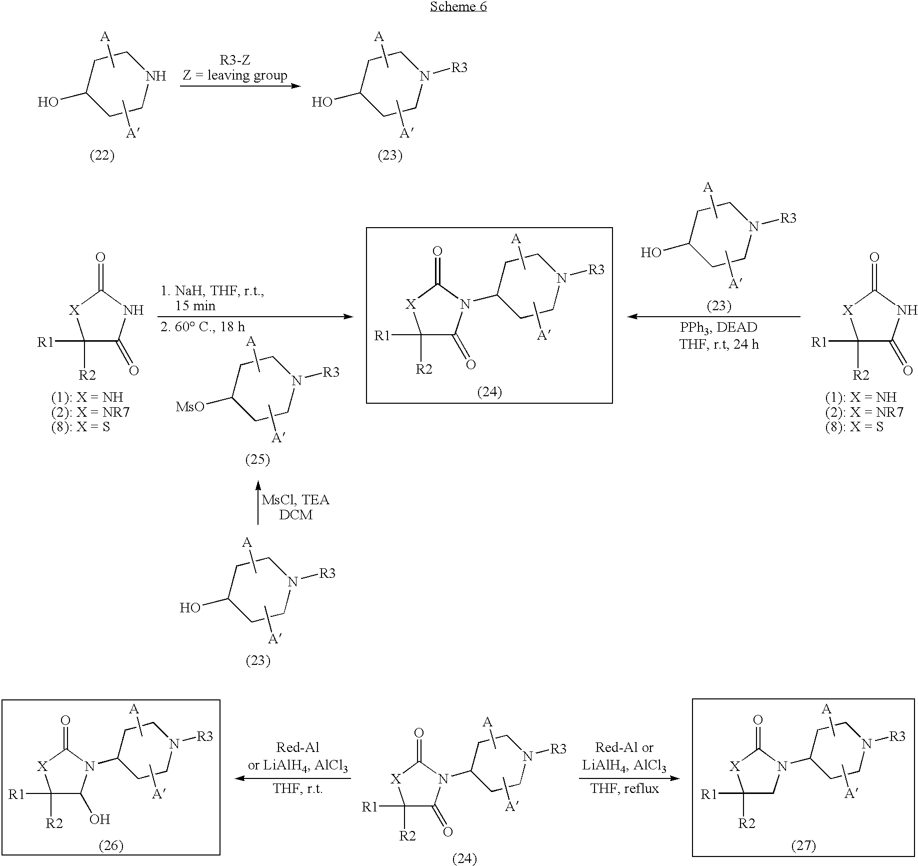

For final compounds in which X═N—R7, an alternative synthetic pathway can be adopted as described in Scheme 7, in order to introduce substituent R7 at a later stage of the synthesis.

Compounds (28), which can be considered as final compounds and can be prepared as described in the previous Scheme 6 (for X═NH), can undergo alkylation or arylation or acylation or sulfonylation reactions with a suitable reagent R7-Z (where Z is a suitable leaving group). Final compounds (29) thus obtained can be further reacted to give final compounds (30) and (31), as described in the previous Scheme 4.

Moreover, all the final compounds (29), (30), (31) can be further functionalized as ammonium salts, quaternary ammonium salts or N-oxide derivatives as described in the previous Scheme 5.

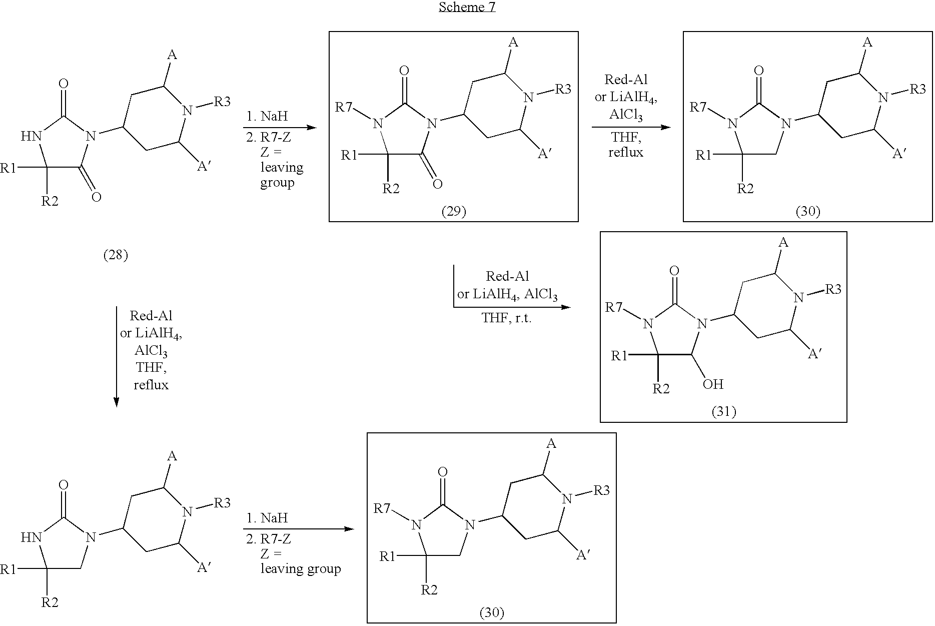

Alternatively to what described in Schemes 1 and 2, a different synthetic pathway can be adopted when Y═CH₂ for the synthesis of intermediates (37) and (38), as described in the following Scheme 8.

Amino acid primary amides (4) were prepared as described in Scheme 1.

The primary amide group can be reduced to primary amino group by reaction conditions described in the literature (Challis, B. C. et al., The Chemistry of Amides, 1970, Wiley, 795; Brown, H. C. et al., Tetr. 1992, 41, 996), for example with borane-dimethylsulfide complex or with lithium aluminium hydride, to give the intermediate (33). Alternatively, the primary amino group in compounds (4) can be functionalized by reaction with a suitable reagent R7-Z, where Z is a suitable leaving group, to give intermediates (34), which in turn can be reduced to primary amines (35).

Intermediates (33), (35) can then undergo cyclization reaction to give imidazolidinones (37) and (38) respectively. The cyclization step can be realized using procedures described in the literature, for example with carbonyldiimidazole (De Cicco, P. et al., Bioorg. Med. Chem. Lett. 1997, 7(18), 2331-2336) or phosgene or triphosgene (Gawley, R. E. et al., J. Org. Chem. 1996, 61, 8103-8112) as source of the carbonyl group; alternatively, the same cyclization can be achieved in two steps by formation of the corresponding p-nitrophenyl carbamate or carbonate and cyclization with sodium hydroxide (Akiba, T. et al., Tetrahedron 1994, 50(13), 3905-3914).

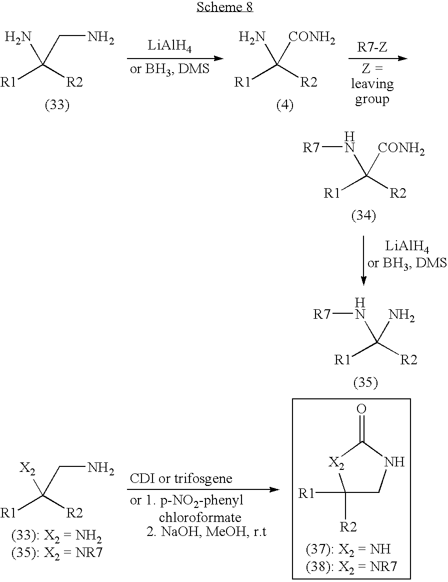

Similarly to what described in Scheme 8, an alternative synthetic pathway can be adopted for final compounds (27) described in Scheme 6. The synthetic pathway is outlined in Scheme 9.

Amino acid amides (40) were prepared as described in the literature (Challis, B. C. et al., The Chemistry of Amides, 1970, Wiley, 795), for example with condensing agents such as dicyclohexylcarbodiimide and 1-hydroxybenzotriazole.

The amide group can be reduced to primary amino group by reaction conditions described in the literature (Challis, B. C. et al., The Chemistry of Amides, 1970, Wiley, 795; Brown, H. C. et al., Tetr. 1992, 41, 996), for example with borane-dimethylsulfide complex or with lithium aluminium hydride, to give intermediate (42). This intermediate can be cyclized, for example with carbonyldiimidazole or trifosgene, to give final compound (44).

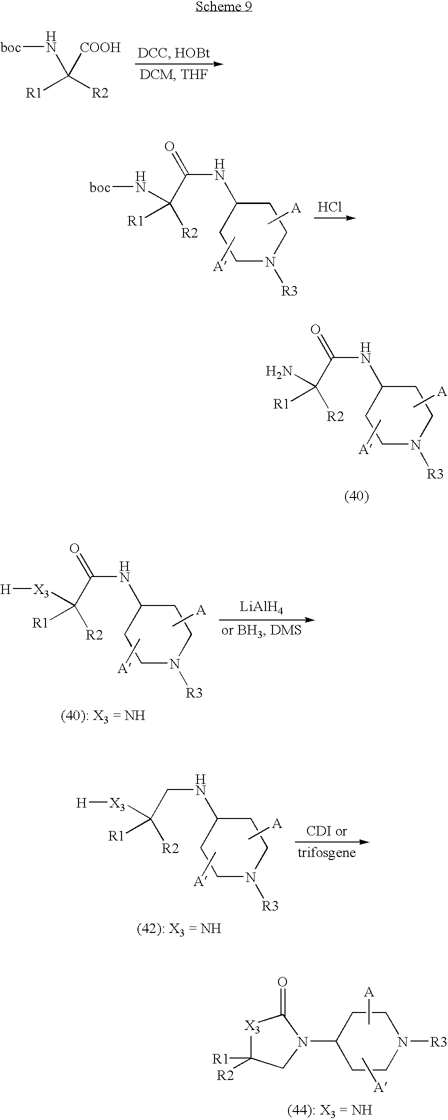

The compounds of formula (I) have antimuscarinic activity and, in particular, they show potent interaction with the M3 subtype. They also show different selectivity with respect to the muscarinic receptors M1 and M2 and can be used for the preparation of pharmaceutical compositions for the treatment of respiratory, urinary or gastrointestinal diseases such as asthma, chronic obstructive pulmonary disease (COPD), chronic bronchitis, cough, emphysema and rhinitis; urinary incontinence, bladder-related diseases; irritable bowel syndrome.

The compound or composition of the invention may be formulated for administration by any route, and is preferably in unit dosage form or in a form that a human patient may administer to himself in a single dosage. Advantageously, the composition is suitable for oral, rectal, topical, parenteral, intravenous or intramuscular administration. Preparations may be designed to give slow release of the active ingredient.

Standard pharmaceutical compositions can be prepared with conventional methods and excipients.

The invention will be hereinafter illustrated in greater detail in the following experimental section.

### EXPERIMENTAL SECTION

The affinity of the compounds of the invention for the muscarinic receptor subtypes M₁, M₂, M₃ was determined by a radioligand binding assay, which was performed as described below:

Cell Lines and Membrane Preparations

CHO—K1 clone cells expressing the human M₁, M₂ or M₃-receptors (Swissprot P11229, P08172, P20309 respectively) were harvested in Ca⁺⁺/Mg⁺⁺ free phosphate-buffered saline and collected by centrifugation at 1500 rpm for 3 min. The pellets were resuspended in ice cold buffer A (15 mM Tris-HCl pH 7.4, 2 mM MgCl₂, 0.3 mM EDTA, 1 mM EGTA) and homogenized by a PBI politron (setting 5 for 15 s). The crude membrane fraction was collected by two consecutive centrifugation steps at 40000 g for 20 min at 4° C., separated by a washing step in buffer A. The pellets obtained were finally resuspended in buffer B (75 mM Tris HCl pH 7.4, 12.5 mM MgCl₂, 0.3 mM EDTA, 1 mM EGTA, 250 mM sucrose), and aliquots were stored at −80° C.

### Radioligand Binding Conditions

The day of experiment, frozen membranes were resuspended in buffer C (50 mM Tris-HCl pH 7.4, 2.5 mM MgCl₂, 1 mM EDTA). The non selective muscarinic radioligand [³H]-N-methyl scopolamine (Mol. Pharmacol. 45:899-907) was used to labelled the M1, M2, and M3 binding sites. Binding experiments were performed in duplicate (ten point concentrations curves) in 96 well plates at radioligand concentration of 0.1-0.3 nM. The non specific binding was determined in the presence of cold N-methyl scopolamine 10 μM. Samples (final volume 0.75 ml) were incubated at room temperature for 120 min for Ml, 60 min for M₂ and 90 min for M₃ binding assay. The reaction was terminated by rapid filtration through GF/B Unifilter plates and two washes (0.75 ml) with cold buffer C using a Packard Filtermate Harvester. Radioactivity on the filters was measured by a microplate scintillation counter TopCount NXT (Camberra Packard).

### PREPARATIVE EXAMPLES

### Procedure 1

### Intermediates (1) Described in Scheme 1

### Synthesis of 5-phenyl-5-cyclohexyl Hydantoin (1a)

Cyclohexyl phenyl ketone (0.564 g, 3 mmoles) is dissolved in 20 mL of a 1:1 mixture of ethanol and water, in a stainless steel sealed tube. Potassium cyanide (0.585 g, 9 mmoles) and ammonium carbonate (3.28 g, 30 mmoles) are added and the mixture is heated at 100° C. for 18 hours.

The reaction mixture is then allowed to cool to room temperature, diluted with 20 mL of water and cooled to 0° C.: the desired product precipitates as a white solid and is filtered to give 0.70 g of pure product.

Hydantoin derivatives (1b)-(1g) were synthesized following the same procedure, starting from the corresponding commercially available ketones. For compounds (1b) and (1c), the starting ketone derivative was prepared as described by Reichard, G. A. et al, Org. Lett. 2003 5(23), 4249-4251.

TABLE 1

| 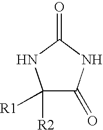 | R1 | R2 | analytical |
|---|---|---|---|
| (1a) | phenyl | cyclohexyl | LC-MS (ESI pos): 259.37 |
| | | | (MH+) |
| (1b) | 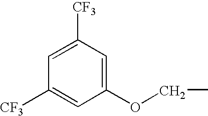 | phenyl | LC-MS (ESI pos): 419.26(MH+) |
| (1d) | benzyl | benzyl | LC-MS (ESI pos): 281.20 |
| (1e) | 4-OMe phenyl | 4-OMe phenyl | LC-MS (ESI pos): 313.10 |
| (1f) | 3-OH phenyl | phenyl | LC-MS (ESI pos): 269.10 |
| (1g) | 2-thienyl | 2-thienyl | LC-MS (ESI pos): 265.18 |

Hydration derivative (1i) was synthesized following the same Procedure 1, starting from the corresponding commercially available ketone.

TABLE 1
bis

| 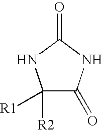 | R1 | R2 | analytical |
|---|---|---|---|
| (1i) | cyclo- | phenyl | LC-MS (ESI pos): 245.28 (MH+) |
| | pentyl | | |

### Procedure 2

### Intermediates (1) Described in Scheme 1

### Synthesis of 5,5-di-(4-fluorophenyl)hydantoin (1h)

Bis-(4-fluorophenyl)-ethandione (1.23 g, 5 mmoles) is dissolved in ethanol (20 mL) and added with urea (0.39 g, 6.5 mmoles) and potassium hydroxide (pellets, 0.476 g, 8.5 mmoles); the resulting mixture is heated at 80° C. for 24 hours. The reaction is allowed to cool to room temperature and diluted with water (40 mL) and cooled to 0° C.: the desired product precipitates as a white solid and is filtered to give 0.62 g of pure product.

TABLE 2

| 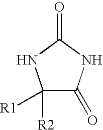 | R1 | R2 | analytical |
|---|---|---|---|
| (1h) | 4-F phenyl | 4-F phenyl | LC-MS (ESI pos): |
| | | | 389.22 (MH+) |

Hydration derivative (1j) was synthesized following the same Procedure 2, starting from the corresponding commercially available ketone.

TABLE 2
bis

| 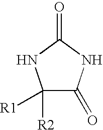 | R1 | R2 | analytical |
|---|---|---|---|
| (1j) | 3-F | 3-F | LC-MS (ESI pos): 389.21 (MH+) |
| | phenyl | phenyl | |

### Procedure 3

### Intermediates (2) Described in Scheme 1

### Synthesis of 1-benzyl-5-phenyl Hydantoin (2a)

### 1. N-benzyl Phenyl Glycine Amide

N-benzyl phenyl glycine ethyl esther (prepared as described by Browne, L. et al, J. Org. Chem. 1952, 17, 1187-1190) in the amount of 0.57 g (2.1 mmoles) is dissolved in ethanol (20 mL) and added to a 30% aqueous solution of ammonia (8 mL). The mixture is heated at 60° C. for 8 hours, then the solvent is evaporated and the product is obtained as an oil (0.51 g) which is employed without further purification in the next step.

### 2. N-benzyl-N-(4-Nitrophenyl Carbamoyl)Phenylglycine Amide

N-benzyl phenyl glycine amide (0.51 g, 2.08 mmoles) is dissolved in dry THF (15 mL) under nitrogen atmosphere. N-methyl morpholine is added, the reaction mixture is cooled to 0° C. and 4-nitrophenyl chloroformate (642 mg, 3.18 mmoles) is added. The reaction is stirred at room temperature for 2 hours; the solvent is evaporated under vacuum, the residue is dissolved in ethyl acetate and extracted with water, then with brine, finally dried and concentrated under vacuum to give an orange oil (0.82 g) which is submitted to the next step without further purification.

### 3.1-benzyl-5-phenyl Hydantoin (2a)

N-benzyl-N-(4-nitrophenyl carbamoyl)phenyl glycine amide (0.82 g, 2.02 mmoles) is dissolved in methanol (20 mL) and 3 mL of a 2M solution of sodium hydroxide are added. The mixture is stirred at room temperature for 2 hours.

The solvent is evaporated under vacuum, the residue is dissolved in ethyl acetate and extracted with a saturated NaHCO₃ solution, water, then with brine, finally dried and concentrated under vacuum to give a yellow solid which is crystallized from methanol to give the desired product as a white solid (0.27 g).

TABLE 3

| 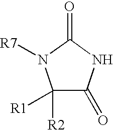 | R1 | R2 | R7 | analytical |
|---|---|---|---|---|
| (2a) | phenyl | H | benzyl | LC-MS (ESI pos): |
| | | | | 267.10 (MH+) |

### Procedure 4

### Intermediates (11) Described in Scheme 3 and Final Compounds (24) Described in Scheme 6

### Synthesis of 3-(1-benzyl-piperidin-4-yl)-5,5-diphenyl-imidazolidine-2,4-dione (24a)

Commercially available 5,5-diphenyl hydantoin (phenyloin, 0.50 g, 1.98 mmoles) is dissolved in dry THF (20 mL); triphenyl phosphine (0.78 g, 2.97 mmoles) and 4-hydroxy-N-benzyl piperidine (0.567 g, 2.97 mmoles) are added to the reaction mixture. The resulting solution is cooled to 0° C. and diethyl aza-dicarboxylate (DEAD, 0.47 mL) is added dropwise. The reaction is then stirred at room temperature for 24 hours. The solvent is evaporated under vacuum and the product is purified by chromatography on silica gel (500 g, eluent: AcOEt:hexane=2:8 to AcOEt), to yield 0.8 g of pure product as a white solid.

Compounds described in Table 4 were synthesized following the same procedure, starting from the corresponding hydantoins or oxazolidine-2,4-diones.

TABLE 4

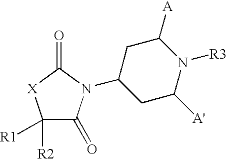

| Starting | | | | | | | | |
|---|---|---|---|---|---|---|---|---|
| material | Product | R1 | R2 | X | R3/PG | A | A′ | analytical |
| phenytoin | (24a) | phenyl | phenyl | NH | benzyl | H | H | MS (EI pos): M+. 425.13, 334.16 |
| | | | | | | | | ¹H-NMR (DMSO − 343 K): |
| | | | | | | | | 9.45 (s br, 1 H); |
| | | | | | | | | 7.53-7.32 (m, 15 H); |
| | | | | | | | | 4.25 (s, br, 2 H); 4.20 (m, 1 H); |
| | | | | | | | | 3.40 (m, 2 H); 3.10 (m, 2 H); |
| | | | | | | | | 2.58 (dq, 2 H); 1.87 (m, 2 H). |
| phenytoin | (24b) | phenyl | phenyl | NH | methyl | H | H | LC-MS (ESI pos): 350.10 (MH+) |
| (1a) | (24c) | phenyl | cyclohexyl | NH | benzyl | H | H | LC-MS (ESI pos): 432.10 (MH+) |
| (1b) | (24d) | 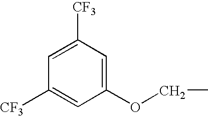 | phenyl | NH | benzyl | H | H | MS (EI pos): (M+.) 591.23,500.24, 243.03¹H-NMR (DMSO − 373 K): 8.73 (s,1 H); 7.67-7.60 (m, 2 H);7.60-7.54 (m, 3 H);7.49-7.34 (m, 8 H); 4.82 (d, 1 H);4.53 (d, 1 H); 4.22-3.90 (m,2 H); 3.36-3.18 (m, 3 H);2.83-2.39 (m, 4 H); 1.88-1.66 (m, 2 H). |
| (1d) | (24e) | benzyl | benzyl | NH | benzyl | H | H | LC-MS (ESI pos): 454.10 (MH+) |
| (1e) | (24f) | 4-OMe phenyl | 4-OMe phenyl | NH | benzyl | H | H | LC-MS (ESI pos): 486.03 (MH+) |
| (1f) | (24g) | 3-OH phenyl | phenyl | NH | benzyl | H | H | LC-MS (ESI pos): 442.04 (MH+) |
| (1h) | (24h) | 4-F phenyl | 4-F phenyl | NH | benzyl | H | H | LC-MS (ESI pos): 462.35 (MH+) |
| (1g) | (24i) | 2-thienyl | 2-thienyl | NH | benzyl | H | H | LC-MS (ESI pos): 438.00 (MH+) |
| (2a) | (24j) | phenyl | H | N-benzyl | benzyl | H | H | LC-MS: 440.2 (MH+) |
| | | | | | | | | ¹H-NMR (CDCl3): |
| | | | | | | | | 7.45-7.27 (m, 10 H); |
| | | | | | | | | 7.17-7.08 (m, 5 H); |
| | | | | | | | | 5.11 (d, 1 H); 4.58 (s br, 1 H); |
| | | | | | | | | 4.00 (m, 1 H); 3.71 (d, 1 H); 3.54 (m, |
| | | | | | | | | 2 H); 2.99 (m, 2 H); 2.55 (m, 2 H); |
| | | | | | | | | 2.09 (m, 2 H); 1.66 (m, 2 H). |

| | | | |
|---|---|---|---|
| phenytoin | (24k) | 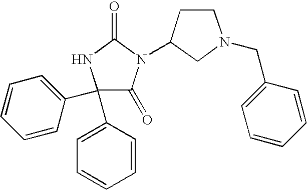 | LC-MS (ESI pos): 412.18 (MH+) |
| phenytoin | (24p) | 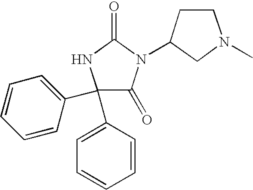 | MS (ESI POS): 336.39 (MH+) |
| phenytoin | (24q) | 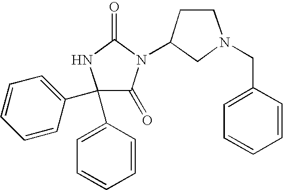 | MS (ESI POS): 412.2 (MH+) |
| phenytoin | (24r) | 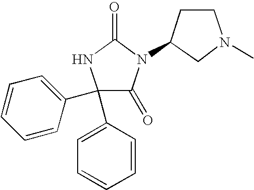 | MS (ESI POS): 336.39 (MH+) |
| phenytoin | (24s) | 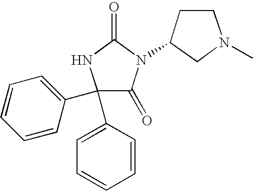 | MS (ESI POS): 336.39 (MH+) |
| phenytoin | (24t) | 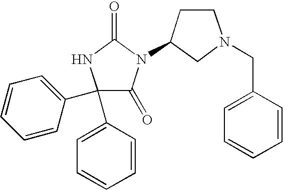 | MS (ESI POS): 412.2 (MH+) |
| phenytoin | (24u) | 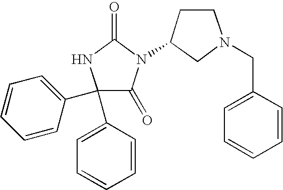 | MS (ESI POS): 412.2 (MH+) |
| (1h) | (24v) | 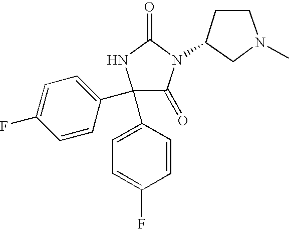 | MS (ESI POS): 371.26 (MH+) |
| (1i) | (24w) | 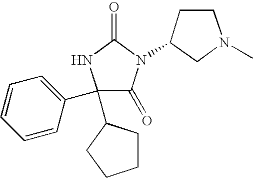 | MS (ESI POS): 327.18 (MH+) |
| phenytoin | (24x) | 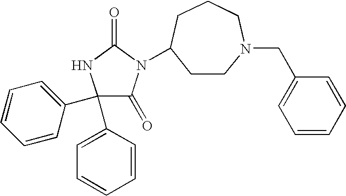 | MS (ESI POS): 439.35 (MH+) |
| (1j) | (24y) | 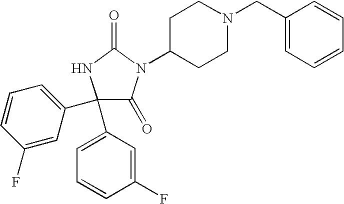 | MS (ESI POS): 462.20 (MH+) |
| (1i) | (24z) | 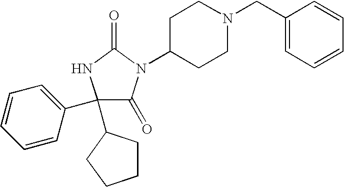 | MS (ESI POS): 417.24 (MH+) |
| (1h) | (24aa) | 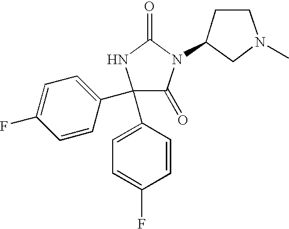 | MS (ESI POS): 371.26 (MH+) |
| phenytoin | (24ab) | 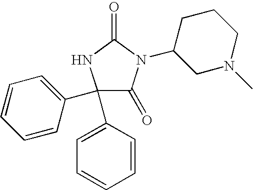 | MS (ESI POS): 350.28 (MH+) |

### Procedure 5

### Intermediates (11) Described in Scheme 3 and Final Compounds (24) Described in Scheme 6

### 1. synthesis of methanesulfonic acid 8-methyl-8-aza-bicyclo[3.2.1]oct-3-yl ester (tropine mesilate)

Tropine (0.150 g, 1.063 mmoles) is added to a solution of triethyl amine (0.207 mL, 1.5 mmoles) in dry DCM (10 mL; the resulting mixture is cooled to 0° C. and mesyl chloride (0.099 mL, 1.276 mmoles) is added. The reaction is stirred at 0° C. for 1 hour, then the solvent is evaporated in vacuum and the product is obtained as a white solid which is employed in the next step without purification.

### 2. Synthesis of 3-(8-Methyl-8-aza-bicyclo[3.2.1]oct-3-yl)-5,5-diphenyl-imidazolidine-2,4-dione (241)

Commercially available 5,5-diphenyl hydantoin (phenyloin, 0.20 g, 0.793 mmoles) is dissolved in dry DMF (10 mL) and K₂CO₃ (0.33 g, 2.38 mmoles) and tropine mesilate (prepared in the previous step), dissolved in dry DMF (3 mL) are added. The reaction is then stirred at 80° C. for 18 hours. The reaction mixture is diluted with water and extracted with ethyl acetate; the organic phase is washed with water, then with brine, finally dried and concentrated under vacuum to give a yellowish solid which is crystallized from ethyl ether to give the desired product as a white solid (0.18 g).

Compounds described in Table 5 were synthesized following the same procedure, starting from the corresponding hydantoins.

TABLE 5

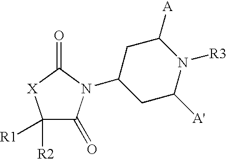

| | | | | | | | | |
|---|---|---|---|---|---|---|---|---|
| Starting | | | | | | | | |
| material | Product | R1 | R2 | X | R3/PG | A | A′ | analytical |

| | | | | | | | |
|---|---|---|---|---|---|---|---|
| phenytoin | (24l) | phenyl | phenyl | NH | methyl | CH₂CH₂ | LC-MS: 376.1 (MH+) |
| | | | | | | | ¹H-NMR ‘(CDCl3): 7.41-7.29 |
| | | | | | | | (m, 10 H); 5.89 (s br, |
| | | | | | | | 1 H); 4.45 (m, 1 H); |
| | | | | | | | 3.53 (m, 2 H); 2.72 (m, 2 H); 2.72 |
| | | | | | | | (s, 3 H); 2.33 (m, 2 H); |
| | | | | | | | 1.82 (m, 2 H); 1.27 (m, 2 H). |
| (1b) | (24m) | 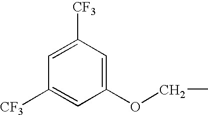 | phenyl | NH | methyl | CH₂CH₂ | MS (ESI pos): 542.1 (MH+)¹H-NMR (CDCl3 +Na2CO3— +D2O − 333 K): 7.63-7.32 (m, 8 H); 3.2 (s br,2 H); 2.66-2.49 (m, 2 H);2.41 (s, 3 H); 2.15-1.97 (m,3 H); 1.72-1.61 (m,2 H); 1.44-1.22 (m, 4 H). |
| (1c) | (24n) | 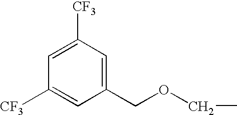 | phenyl | NH | methyl | CH₂CH₂ | MS (ESI pos): 556.13 (MH+)¹H-NMR (CDCl3): 7.79 (s, 1 H);7.65 (s, 2 H); 7.56-7.48 (m, 2 H); 7.44-7.33 (m,3 H); 5.75 (s, 1 H); 4.62(dd, 2 H); 4.50-4.34 (m, 1 H);4.12 (d,1 H); 3.78 (d,1 H); 3.23-3.09 (m, 2 H);2.31-2.02 (m, 4 H); 2.16(s, 3 H); 1.90-1.59 (m, 4 H). |
| (2^{o}) | (24o) | phenyl | H | N- | methyl | CH₂CH₂ | LC-MS: 390.1 (MH+) |
| | | | | benzyl | | | ¹H-NMR (CDCl3): |
| | | | | | | | 7.43-7.27 (m, 6 H); 7.17-7.07 |
| | | | | | | | (m, 4 H); 5.10 (d, 1 H); |
| | | | | | | | 4.58 (s, 1 H); 4.40 (m, 1 H); |
| | | | | | | | 3.73 (d, 1 H); 3.35 (m, 2 H); |
| | | | | | | | 2.63 (m, 2 H); 2.57 (s, |
| | | | | | | | 3 H); 2.14 (m, 2 H); 1.70 |
| | | | | | | | (m, 2 H); 1.44 (m, 2 H). |
| phenytoin | (24 pp) | phenyl | phenyl | NH | methyl | 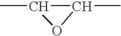 | LC-MS: 390.2 (MH+) |

### Procedure 6

### Intermediates (16) Described in Scheme 4 and Final Compounds (27) Described in Scheme 6

### Synthesis of 3-(1-benzyl-piperidin-4-yl)-5 5-diphenyl-imidazolidin-2-one (27a)

Compound (24a) (1.0 g, 2.3 mmoles) is dissolved in dry THF (20 mL) under nitrogen atmosphere. The solution is cooled to 0° C. and a 3.5 M solution of sodium bis-(2-methoxyethoxy)aluminium hydride (Red-Al) in toluene (5.37 mL, 18.4 mmoles) is added. The mixture is then heated to 85° C. for 4 hours. The reaction is cooled again to 0° C. and quenched with water (5 mL); then 2M sodium hydroxide is added (10 mL) and the mixture is extracted with ethyl acetate; the organic phase is washed with water, then with brine, finally dried and concentrated under vacuum to give a yellowish solid which is crystallized from acetone to give the desired product as a white solid (0.80 g).

Compounds described in Table 6 were synthesized following the same procedure, starting from the corresponding hydantoins.

### Procedure 7

### Intermediates (16) Described in Scheme 4 and Final Compounds (27) Described in Scheme 6

### Synthesis of 1-benzyl-3-(1-benzyl-piperidin-4-yl)-5-phenyl-imidazolidin-2-one (27r)

Lithium aluminium hydride (0.309 g, 8.16 mmoles) is suspended in dry THF (15 ml) under nitrogen atmosphere. The suspension is cooled to 0° C. and a solution of aluminium trichloride (1.085 g, 8.16 mmoles) in dry THF (10 mL) is added. The resulting mixture is stirred at 0° C. for 30 minutes. A solution of compound (24o) (0.5 g, 2.04 mmoles) in dry THF (12 mL) is then added to the mixture of LiAlH₄+AlCl₃: the resulting suspension is heated at 65° C. for 3 hours. The reaction is cooled again to 0° C. and quenched with water (5 mL); then 1M sodium hydroxide is added (10 mL) and the mixture is extracted with ethyl acetate; the organic phase is washed with water, then with brine, finally dried and concentrated under vacuum to give a yellowish solid which is crystallized from acetone to give the desired product as a white solid (0.310 g).

Compounds described in Table 7 were synthesized following the same procedure, starting from the corresponding hydantoins.

TABLE 6

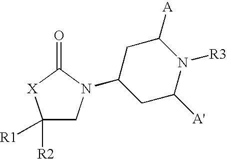

| Starting | | | | | | | | |
|---|---|---|---|---|---|---|---|---|
| material | Product | R1 | R2 | X | R3/PG | A | A′ | analytical |
| (24a) | (27a) | phenyl | phenyl | NH | benzyl | H | H | MS (ESI POS): 412.1 (MH+) |
| | | | | | | | | NMR (CDCl3 + D2O): 7.38-7.19 (m, 15 H); |
| | | | | | | | | 3.94 (s, 2 H); 3.85 (m, 1 H); 3.49 (s, 2 H); 2.93 |
| | | | | | | | | (m, 2 H); 2.09 (m, 2 H); 1.78-1.68 (m, 4H). |
| (24b) | (27b) | phenyl | phenyl | NH | methyl | H | H | MS (ESI POS): 336.2 (MH+) |
| | | | | | | | | ¹H-NMR (DMSO − 343 K): 7.80 (s br, 1 H); |
| | | | | | | | | 7.40-7.21 (m, 10 H); 3.91 (s, 2 H); 3.80 (m, 1 H); |
| | | | | | | | | 3.41 (m, 2 H); 3.09 (m, 2 H); 2.72 (s, 3 H); 2.01 |
| | | | | | | | | (m, 2 H); 1.80 (m, 2 H). |
| (24c) | (27c) | phenyl | cyclohexyl | NH | benzyl | H | H | MS (EI, 70 eV, 150° C., 50-270° C.): |
| | | | | | | | | ¹H-NMR (CDCl3): 7.40-7.19 (m, 10 H); 4.75 (s, |
| | | | | | | | | 1 H); 3.80 (m, br, 1 H); 3.75 (d, 1 H) 3.52 (m br, 1 H); |
| | | | | | | | | 3.47 (d, 1 H); 2.94 (m br, 2 H); 2.13 (m br, 2 H); 1.86- |
| | | | | | | | | 1.52 (m, 8 H); 1.37-1.02 (m, 7 H); 0.78 (m, 1 H). |
| (24e) | (27e) | benzyl | benzyl | NH | benzyl | H | H | MS (EI pos): 439.3 (M+.) |
| | | | | | | | | ¹H-NMR (CDCl3 − 338 K): 7.35-7.10 (m, 15 H); 4.23 |
| | | | | | | | | (s, 1 H); 357-3.39 (m br, 3 H); 3.26 (s, 2 H); 2.87-2.73 |
| | | | | | | | | (m, 6 H); 2.15-1.84 (m, 2 H); 1.33-1.20 (m, 4 H). |
| (24g) | (27g) | 3-OH phenyl | phenyl | NH | benzyl | H | H | |
| (24h) | (27h) | 4-F phenyl | 4-F phenyl | NH | benzyl | H | H | MS (ESI POS): 448.14 (MH+) |
| | | | | | | | | ¹H-NMR (CDCl3): 7.33-7.19 (m, 7 H); 6.99- |
| | | | | | | | | 6.93 (m, 4 H); 4.74 (s br, 1 H); 3.93 (s, 2 H); |
| | | | | | | | | 3.85 (m, 1 H); 3.49 (s, 2 H); 2.94 (m, 2 H); 2.08 |
| | | | | | | | | (m, 2 H); 1.77-1.64 (m, 4 H) |
| (24i) | (27i) | 2-thienyl | 2-thienyl | NH | benzyl | H | H | MS (ESI POS): MH + 424.0 |
| | | | | | | | | ¹H-NMR (CDCl3): 7.33-7.19 (m, 7 H); 6.99- |
| | | | | | | | | 6.93 (m, 4 H); 4.74 (s br, 1 H); 3.93 (s, 2 H); |
| | | | | | | | | 3.85 (m, 1 H); 3.49 (s, 2 H); 2.94 (m, 2 H); 2.08 |
| | | | | | | | | (m, 2 H); 1.77-1.64 (m, 4 H) |
| (24k) | (27k) | 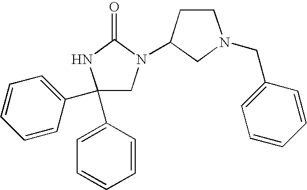 | | | | | | MS (ESI POS): MH + 398.17¹H-NMR (CDCl3): 7.39-7.17 (m, 15 H);4.63 (m, 1 H); 4.11 (d, 1 H); 3.99 (d, 1 H);3.66 (d, 1 H); 3.49 (d, 1 H); 2.88 (ddd, 1 H);2.68 (dd, 1 H); 2.46 (dd, 1 H); 2.27 (dt, 1 H);2.15 (m, 1 H); 1.86-1.71 (m, 1 H) |
| (24f) | (27f) | 4-OMe phenyl | 4-OMe | NH | benzyl | H | H | MS (ESI POS): MH + 472.1 |
| | | | phenyl | | | | | ¹H-NMR (CDCl3 + D2O + Na2CO3): 7.36-7.20 |
| | | | | | | | | (m, 5 H); 7.14 (d, 4 H); 6.84 (d, 4 H); 3.88 (s, 2 H); |
| | | | | | | | | 3.84 (m, 1 H); 3.79 (s, 6 H); 3.49 (s, 2 H); 2.92 |
| | | | | | | | | (m, 2 H); 2.09 (m, 2 H); 1.77-1.66 (m, 4 H). |

| | | | | | | | |
|---|---|---|---|---|---|---|---|
| (24l) | (27l) | phenyl | phenyl | NH | methyl | CH₂CH₂ | MS (ESI POS): 362.16 (MH+) |
| | | | | | | | ¹H-NMR (CDCl3): 7.38-7.22 (m, 10 H) 5.03 (s br, |
| | | | | | | | 1 H); 4.06 (m, 1 H), 3.91 (s, 2 H) 3.26 (m, 2 H); 2.44 (m, |
| | | | | | | | 2 H); 2.29 (s, 3 H); 2.02 (m, 2 H); 1.92-1.44 (m, 4 H). |
| (24 pp) | (27 pp) | phenyl | phenyl | NH | methyl | 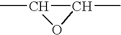 | MS (ESI POS): 376.15 (MH+)¹H-NMR (CDCl3 + D2O + Na2CO3): 7.39-7.16 (m, 10 H); 4.24 (m, 1 H); 3.86 (s, 2 H);3.44 (s, 2 H); 3.21 (d, 2 H); 2.50 (s, 3 H);2.13 (ddd, 2 H); 1.19 (dd, 2 H). |

| | | | |
|---|---|---|---|
| (24p) | (27p) | 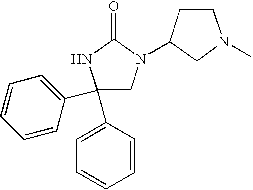 | MS (ESI POS): 322.39 (MH+) |
| (24q) | (27q) | 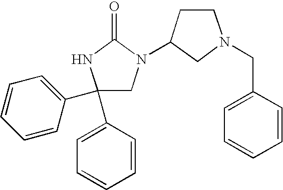 | MS (ESI POS): 398.17 (MH+)1 H NMR (CDCl3 + D2O + Na2CO3): 7.40-7.18 (m, 15 H); 4.63 (m, 1 H); 4.11 and3.99 (ABq, 2 H); 3.66 and 3.50 (ABq, 2 H);2.88 (ddd, 1 H); 2.68 (dd, 1 H); 2.46 (dd, 1 H);2.28 (ddd, 1 H); 2.17 (ddd, 1 H); 1.79 (m, 1 H). |
| (24r) | (27r) | 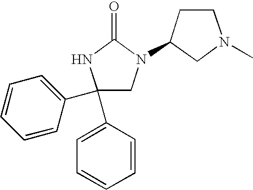 | MS (ESI POS): 322.39 (MH+) |
| (24s) | (27s) | 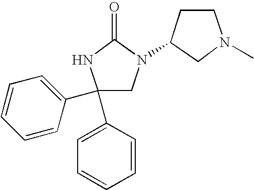 | MS (ESI POS): 322.39 (MH+) |
| (24t) | (27t) | 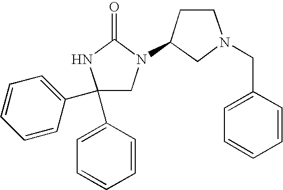 | MS (ESI POS): 397.9 (MH+)1 H NMR (CDCl3 + D2O + Na2CO3): 7.39-7.19 (m, 15 H); 4.62 (m, 1 H); 4.11-3.99 (ABq,2 H); 3.66-3.49 (ABq, 2 H); 2.88 (ddd, 1 H);2.68 (dd, 1 H); 2.46 (dd, 1 H); 2.34-2.09 (m, 2 H);1.78 (m, 1 H). |
| (24u) | (27u) | 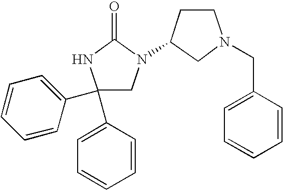 | MS (ESI POS): 397.9 (MH+)1 H NMR ‘(1 H CDCl3 + D2O + Na2CO3):7.38-7.19 (m, 15 H); 4.62 (m, 1 H); 4.11-3.99 (ABq, 2 H); 3.66-3.49 (ABq, 2 H);2.88 (ddd, 1 H); 2.68 (dd, 1 H); 2.46 (dd, 1 H);2.34-2.09 (m, 2 H); 1.78 (m, 1 H). |
| (24v) | (27v) | 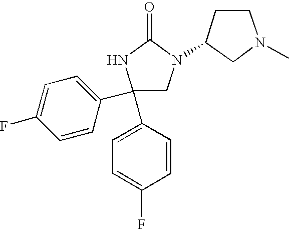 | MS (ESI POS): 358.32 (MH+) |
| (24w) | (27w) | 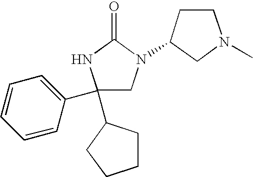 | MS (ESI POS): 313.34 (MH+) |
| (24x) | (27x) | 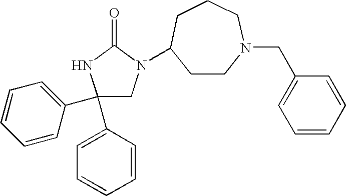 | MS (ESI POS): 426.2 (MH+)1 H NMR (CDCl3 + D2O + Na2CO3 328 K):7.38-7.16 (m, 10 H); 4.13 (m, 1 H); 3.96 (s, 2 H);3.95-3.86 (m, 2 H); 3.69 (m, 1 H); 3.62 (s, 2 H);3.47-3.18 (m, 2 H); 2.77-2.56 (m, 4 H); 2.05-1.47 (m, 6 H). |
| (24y) | (27y) | 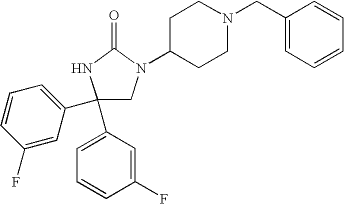 | MS (ESI POS): 447.9 (MH+)1 H NMR (CDCl3 + D2O + Na2CO3): 7.35-7.20 (m, 8 H); 7.03-6.92 (m, 5 H); 3.90 (s, 2 H);3.85 (m, 1 H); 3.49 (s, 2 H); 2.94 (m, 2 H);2.09 (m, 2 H); 1.78-1.67 (m, 4 H). |
| (24z) | (27z) | 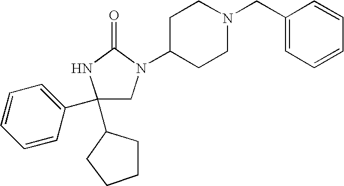 | MS (ESI POS): 404.3 (MH+)1 H NMR (1 H CDCl3 + D2O + Na2CO3): 7.37-7.18 (m, 10 H); 3.78 (m, 1 H); 3.68 (d, 1 H);3.47 (s, 2 H); 3.45 (d, 1 H); 2.89 (m, 2 H);2.46 (m, 1 H); 2.13-1.97 (m, 2 H); 1.80-1.18 (m,11 H); 1.03-0.82 (m, 1 H). |
| (24aa) | (27aa) | 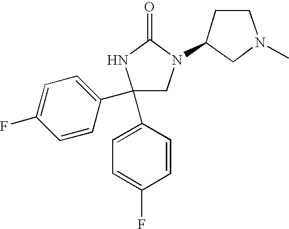 | MS (ESI POS): 358.32 (MH+) |
| (24ab) | (27ab) | 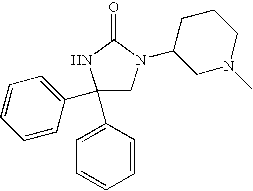 | MS (ESI POS): 336.41 (MH+) |

TABLE 7

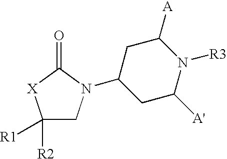

| Starting | | | | | | | | |
|---|---|---|---|---|---|---|---|---|
| material | Product | R1 | R2 | X | R3/PG | A | A′ | analytical |
| (24j) | (27j) | phenyl | H | N-benzyl | benzyl | H | H | MS (ESI POS): 426.1 (MH+) |
| | | | | | | | | ¹H-NMR (CDCl3 − D2O − Na2CO3): 7.39- |
| | | | | | | | | 7.06 (m, 15 H); 4.89 (d, |
| | | | | | | | | 1 H); 4.28 (dd, 1 H); 3.89 |
| | | | | | | | | (m, 1 H); 3.59 (d, 1 H); |
| | | | | | | | | 3.56 (d, 1 H); 3.49 (s, |
| | | | | | | | | 2 H); 3.13 (dd, 1 H); 2.93 (m, |
| | | | | | | | | 2 H); 2.11 (dt, 2 H); |
| | | | | | | | | 1.76-1.61 (m, 4 H). |
| (24o) | (27o) | phenyl | H | N-benzyl | methyl | CH₂CH₂ | | MS (ESI pos): 376.2 (MH+) |
| | | | | | | | | ¹H-NMR (CDCl3 + |
| | | | | | | | | Na2CO3 + D2O): 7.42-7.05 |
| | | | | | | | | (m, 10 H); 4.87 (d, 1 H); 4.34-4.13 (m, 2 H); |
| | | | | | | | | 3.65-3.49 (m, 2 H); |
| | | | | | | | | 3.28-3.02 (m, 3 H); 2.25 (s, |
| | | | | | | | | 3 H); 2.12-1.99 (m, 2 H); |
| | | | | | | | | 1.85-1.48 (m, 6 H). |
| (24d) | (27d) | 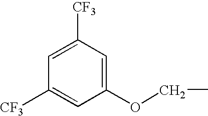 | phenyl | NH | benzyl | H | H | MS (ESI pos): 578.1 (MH+)¹H-NMR (CDCl3 + Na2CO3 +D2O): 7.55-7.18(m, 13 H); 4.28 (dd, 2 H); 3.9-3.78 (m, 1 H);3.75 (d, 1 H); 3.55 (d, 1 H);3.47 (s, 2 H); 3.0-2.83 (m, 2 H); 2.20-1.99(m, 2 H); 1.81-1.52(m, 4 H). |

### Procedure 8

### Intermediates (15) Described in Scheme 4 and Final Compounds (26) Described in Scheme 6

### Synthesis of 3-benzyl-1-(8-methyl-8-aza-bicyclo[3.2.1]oct-3-yl)-4-phenyl-1,3-dihydro-imidazol-2-one (26o)

Compound (24o) (1.0 g, 2.3 mmoles) is dissolved in dry THF (20 mL) under nitrogen atmosphere. The solution is cooled to 0° C. and a 3.5 M solution of sodium bis-(2-methoxyethoxy)aluminium hydride (Red-Al) in toluene (5.37 mL, 18.4 mmoles) is added. The mixture is then stirred at room temperature for 24 hours. The reaction is cooled again to 0° C. and quenched with water (5 mL); then 1M hydrochloric acid is added (10 mL) and the mixture is stirred at room temperature for 30 min. Sodium hydroxide 2M (15 mL) is added to the reaction mixture, which is then extracted with ethyl acetate; the organic phase is washed with water, then with brine, finally dried and concentrated under vacuum to give a yellowish oil which is purified by reverse phase chromatography (C-18 coated SiO₂, 500 g; eluent: MeOH:H₂O=1:1) to give the desired product as a white solid (0.262 g).

TABLE 8

| Starting | | | |
|---|---|---|---|
| material | Product | structure | analytical |
| (24o) | (26o) | 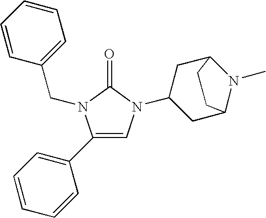 | MS (EI pos): 374.3 (MH+)1 H-NMR (CDCl3 + Na2CO3 −338 K); 7.58-6.91 (m, 10 H); 6.33 (s,1 H); 4.89 (s, 2 H); 3.31-3.02 (m,3 H); 2.35 (s, 3 H); 2.53-2.20 (m,2 H); 2.19-0.78 (m, 6 H). |
| (24i) | (26i) | 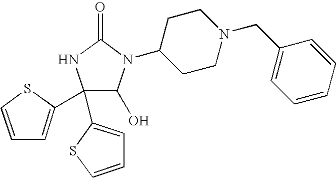 | MS (EI pos): 440.05 (MH+)¹H-NMR (CDCl3): 7.35-7.19 (m,8 H); 7.12-7.02 (m, 3 H); 6.98 (m,1 H); 5.51 (s, 1 H); 5.23 (s, 1 H); 3.78(m, 1 H); 3.49 (s, 2 H); 2.93 (m, 2 H);2.15-1.87 (m, 4 H); 1.71 (m,2 H). |

### Procedure 9

### Intermediates (17) Described in Scheme 5

### Synthesis of 4,4-diphenyl-1-piperidin-4-yl-imidazolidin-2-one (17a)

Compound (27a) (0.50 g, 1.2 mmoles) is dissolved in methanol (15 mL) and water (5 mL). Hydrochloric acid 37% (0.5 mL) and palladium on charcoal (0.20 g) are added and the solution is hydrogenated in a Parr apparatus (H₂: 20 psi) at room temperature for 8 hours. The catalyst is filtered and the clear solution is evaporated to yield the pure product as a white solid (0.380 g).

The other compounds described in Table 9 were synthesized following the same Procedure 9, starting from the corresponding tertiary benzylamines.

TABLE 9

| Starting | | | |
|---|---|---|---|
| material | Product | | analytical |
| (27a) | (17a) | 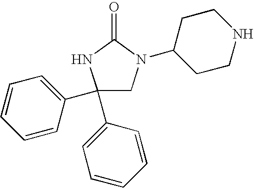 | MS (ESI POS) 322.18 (MH+)NMR(CDCl3 + D2O + Na2CO3):7.37-7.21 (m, 10 H); 3.94 (s,2 H); 3.91 (tt, 1 H); 2.11 (m,2 H); 2.69 (ddd, 2 H); 1.76 (m,2 H); 1.58 (dq, 2 H). |
| (27q) | (17b) | 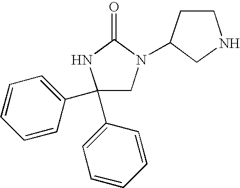 | MS (ESI POS): 307.1 (MH+) |
| (27h) | (17c) | 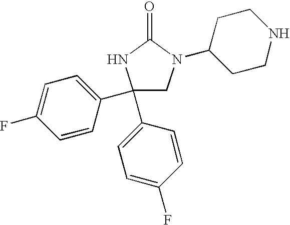 | MS (ESI POS): 358.2 (MH+) |
| (27c) | (17d) | 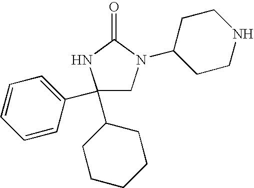 | MS (ESI POS): 328.35 (MH+) |

Compound (17a) can be prepared also following a different procedure, starting from the same intermediate:

### Procedure 10

### Intermediates (17) Described in Scheme 5

### Synthesis of 4,4-diphenyl-1-piperidin-4-yl-imidazolidin-2-one (17a)

Compound (27b) (0.125 g, 0.37 mmoles) is dissolved in dry toluene (20 mL) and added with α-chloroethyl chloroformate (0.075 mL, 0.74 mmoles) and the reaction mixture is refluxed for 3 hours. The solvent is evaporated in vacuum; the residue is dissolved in methanol (20 mL) and added with a 10% NaOH aqueous solution (5 mL). The mixture is heated to 60° C. for 3 hours, then the solvent is evaporated in vacuum. The residue is dissolved in water extracted with ethyl acetate; the organic phase is washed with water, then with brine, finally dried and concentrated under vacuum to give a yellowish solid which is purified by flash chromatography (SiO₂, 60 g; eluent: DCM:MeOH=9:1) to give the desired product as a white solid (0.085 g).

Compounds described in Table 9 bis were synthesized following the same Procedure 10, starting from the corresponding tertiary methyl or benzylamines.

TABLE 9
bis

| Starting | | | |
|---|---|---|---|
| material | Product | structure | analytical |
| (27l) | (17a′) | 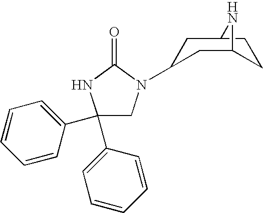 | MS (ESI POS): 348.31 (MH+) |
| (27i) | (17b′) | 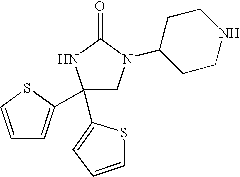 | MS (ESI POS): 334.2 (MH+) |

### Procedure 11

### Final Products (18) Described in Scheme 5

### Synthesis of 1-[1-(3,5-bis-trifluoromethyl-benzyl)-piperidin-4-yl]-4,4-diphenyl-imidazolidin-2-one (18a)

Compound (17a) (0.100 g, 0.311 mmoles) is dissolved in dry DCM (10 mL) and solid K₂CO₃ (0.20 g, 1.45 mmoles) is added. The mixture is vigorously stirred at room temperature and 3,5-bis-trifluoromethyl-benzyl bromide (0.057 mL, 0.311 moles) is added. The reaction is stirred at room temperature for 5 hours, then solid K₂CO₃ is filtered and the solution is evaporated to yield a colorless oil which is crystallized form di-isopropyl ether to afford the pure product as a white solid (0.060 g).

Compounds described in Table 10 were synthesized following the same procedure 11, starting from (17a) or from the intermediate indicated in the table by reaction with the corresponding alkylating agents.

TABLE 10

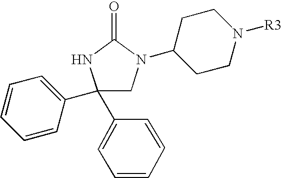

R1 = R2 = phenyl; X = NH; Y = CH₂

| Product | R3 | analytical |
|---|---|---|
| (18a) | 3,5-CF3 benzyl | MS (EI+) M+ = 547.25 |
| | | ¹H-NMR (DMSO − 343 K): 7.95 (s, 2 H); 7.89 (s, 1 H); 7.63 |
| | | (s, 1 H); 7.41-7.18 (m, 10 H); 3.93 (s, 2 H); 3.68 (s, 2 H); |
| | | 3.63-3.48 (m, 1 H); 2.90-2.78 (m, 2 H); 2.20-2.06 (m, 2 H); |
| | | 1.81-1.62 (m, 2 H); 1.60-1.48 (m, 2 H). |
| (18b) | 3-CF3 benzyl | MS (ESI pos) 480.16 (MH+) |
| | | ¹H-NMR (CDCl3 + D2O + Na2CO3): 7.59 (s, 1 H); 7.52- |
| | | 7.20 (m, 13 H); 3.95 (s, 2 H); 3.87 (m, 1 H); 3.53 (s, 2 H); |
| | | 2.93 (m, 2 H); 2.12 (m, 2 H); 1.74 (m, 4 H). |
| (18c) | cyclohexylmethyl | MS (ESI pos) 418.16 (MH+) |
| | | ¹H-NMR (CDCl3 + Na2CO3): 7.37-7.21 (m, 10 H); 4.95 |
| | | (s br, 1 H); 3.95 (s, 2 H); 3.83 (m, 1 H); 2.91 (m, 2 H); |
| | | 2.10 (d, 2 H); 1.98 (m, 2 H); 1.78-1.62 (m, 9 H); 1.44 (m, |
| | | 1 H); 1.30-1.08 (m, 3 H); 0.85 (m, 2 H) |
| (18d) | 2-Cl benzyl | MS (ESI pos): 446.26 (MH+) |
| | | ¹H-NMR (CDCl3): 7.39-7.12 (m, 14 H); 4.88 (s br, 1 H); |
| | | 3.96 (s, 2 H); 3.89 (m, 1 H); 3.61 (s br, 2 H); 2.96 (m, 2 H); |
| | | 2.22 (m, 2 H), 1.76 (m, 4H) |
| (18e) | 3-Cl benzyl | MS (ESI pos) MH + 446.4 |
| | | ¹H-NMR (CDCl3): 7.37-7.20 (m, 14 H); 4.89 (s br, 1 H); |
| | | 3.96 (s, 2 H); 3.87 (m, 1 H); 3.46 (s br, 2 H); 2.91 (m, 2 H); |
| | | 2.10 (m, 2 H), 1.74 (m, 4 H) |
| (18f) | 2-OMe benzyl | MS (ESI pos) MH + 442.3 |
| | | ¹H-NMR (CDCl3): 7.39-7.18 (m, 11 H); 6.98-6.83 (m, 3 H); |
| | | 4.89 (s br, 1 H); 3.96 (s, 2 H); 3.88 (m, 1 H); 3.82 (s, 3 H); 3.60 |
| | | (s br, 2 H); 3.03 (m, 2 H); 2.21 (m, 2 H); 1.83-1.67 (m, 4H) |
| (18g) | 3,4,5-F benzyl | MS (ESI pos) 466.09 (MH+) |
| | | ¹H-NMR (CDCl3): 7.37-7.22 (m, 10 H); 6.95 (m, 2 H); |
| | | 4.91 (s br, 1 H); 3.96 (s, 2 H); 3.87 (m, 1 H); 3.41 (s br, |
| | | 2 H); 2.87 (m, 2 H); 2.12 (m, 2 H); 1.79-1.66 (m, 4 H). |
| (18h) | 3-F benzyl | MS (ESI pos) MH + 430 |
| | | ¹H-NMR (CDCl3 + D2O + Na2CO3): 7.39-7.17 (m, 11 H); |
| | | 7.05 (d, 2 H); 6.92 (dd, 1 H); 3.96 (s, 2 H); 3.86 (m, 1 H); 3.48 |
| | | (s, 2 H); 2.91 (m, 2 H); 2.10 (m, 2 H); 1.84-1.66 (m, 4 H) |
| (18i) | 3-methylbenzyl | MS (ESI pos) 426.18 (MH+) |
| | | ¹H-NMR (CDCl3 + D2O + Na2CO3): 7.37-7.01 (m, 14 H); |
| | | 3.95 (s, 2 H); 3.85 (m, 1 H); 3.45 (s, 2 H); 2.93 (m, 2 H); |
| | | 2.33 (s, 3 H); 2.08 (m, 2 H); 1.80-1.67 (m, 4 H). |
| (18k) | 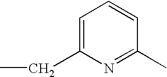 | MS¹H-NMR (CDCl3): 7.51 (dd, 1 H); 7.32-7.17 (m, 11 H);7.00 (d, 1 H); 5.27 (s br, 1 H); 3.92 (s, 2 H); 3.85 (m, 1 H);3.65 (m, 2 H); 2.98 (m, 2 H); 2.50 (s, 3 H); 2.10-2.00 (m,4 H); 1.71 (m, 2 H). |
| (18n) | Phenoxyethyl | MS¹H-NMR (CDCl3 + D2O + Na2CO3): 7.37 (m, 12 H);6.98-6.86 (m, 3 H); 4.08 (t, 2 H); 3.94 (s, 2 H); 3.86 (m,1 H); 3.06 (m, 2 H); 2.80 (t, 2 H); 2.24 (m, 2 H); 1.83-1.71(m, 4 H). |
| (18o) | phenylpropyl | MS (ESI pos) 440.12 (MH+) |
| | | ¹H-NMR (CDCl3 + D2O + Na2CO3): 7.37-7.13 (m, 15 H); |
| | | 3.94 (s, 2 H); 3.84 (m, 1 H); 2.96 (m, 2 H); 2.62 (dd, 2 H); |
| | | 2.36 (dd, 2 H); 2.04 (m, 2 H); 1.85-1.68 (m, 6 H). |
| (18p) | 3-COOMe benzyl | MS (ESI pos) MH + 470.3 |
| | | ¹H-NMR (CDCl3): 7.99 (s, 1 H), 7.37-7.20 (m, 13 H); 4.85 |
| | | (s br, 1 H); 3.96 (s, 2 H); 3.92 (s, 3 H); 3.87 (m, 1 H); 3.54 |
| | | (s br, 2 H); 2.91 (m, 2 H); 2.14 (m, 2 H), 1.74 (m, 4 H) |
| (18q) | acetyl | MS (ESI pos) MH + 364.3 |
| | | ¹H-NMR (CDCl3): 7.37-7.20 (m, 14 H); 4.89 (s br, 1 H); |
| | | 3.96 (s, 2 H); 3.87 (m, 1 H); 3.46 (s br, 2 H); 2.91 (m, 2 H); |
| | | 2.10 (m, 2 H), 1.74 (m, 4 H). |
| (18r) | 3-chlorobenzoyl | MS (ESI pos) 460.04 (MH+) |
| | | ¹H-NMR (CDCl3 + D2O + Na2CO3 333 K): 7.42-7.21 |
| | | (m, 14 H); 4.31 (m, 2 H); 4.07 (tt, 1 H); 3.94 (s, 2 H); 2.98 |
| | | (m, 2 H); 1.82 (m, 2 H); 1.62 (m, 2 H). |
| (18s) | 2-phenyl acetyl | MS (ESI pos) 440.12 (MH+) |
| | | ¹H-NMR (CDCl3 + D2O + Na2CO3): 7.38-7.17 (m, 15 H); |
| | | 4.76 (m, 1 H); 4.00 (m, 1 H); 3.91 (m, 1 H); 3.85 (d, 1 H); |
| | | 3.73 (d, 1 H); 3.73 (s, 2 H); 3.04 (ddd, 1 H); 2.60 (ddd, 1 H); |
| | | 1.78 (m, 1 H); 1.66 (m, 1 H); 1.52 (dq, 1 H); 1.18 (dq, 1 H). |
| (18t) | methansulphonyl | MS (ESI pos) 400.08 (MH+) |
| | | ¹H-NMR (CDCl3 + D2O + Na2CO3): 7.39-7.21 (m, 10 H); |
| | | 3.96 (tt, 1 H); 3.93 (s, 2 H); 3.90 (m, 2 H); 2.78 (s, 3 H); |
| | | 2.77 (ddd, 2 H); 1.86 m, 2 H); 1.78 (dq, 2 H). |
| (18u) | 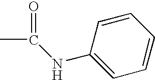 | MS (ESI pos) 441.l1 (MH+)¹H-NMR (CDCl3 + D2O + Na2CO3): 7.38-7.21 (m, 14 H);7.03 (m, 1 H); 4.16 (m, 2 H); 4.07 (tt, 1 H); 3.93 (s, 2 H);2.98 (ddd, 2 H); 1.85 (m, 2 H); 1.68 (dq, 2 H). |
| (18v) | 2-thienyl ethyl | MS (ESI pos) MH + 432.1 |
| | | ¹H-NMR (CDCl3 + D2O + Na2CO3): 7.37-7.21 (m, 10 H); |
| | | 7.16 (dd, 1 H); 6.91 (dd, 1 H); 6.81 (m, 1 H); 3.96 (s, 2 H); 3.87 |
| | | (m, 1 H); 3.00 (m, 4 H); 2.65 (dd, 2 H); 2.16 (m, 2 H); 1.85- |
| | | 1.67 (m, 4 H). |
| (18w) | 2-tetrahydrofuryl | MS (ESI pos) MH + 406.1 |
| | methyl | ¹H-NMR (CDCl3): 7.37-7.20 (m, 10 H); 4.96 (s br, 1 H); 3.99 (m, |
| | | 1 H); 3.93 (s, 2 H); 3.91-3.78 (m, 2 H); 3.72 (m, 1 H); 3.05 (m, 2 H); |
| | | 2.48 (dd, 1 H); 2.37 (dd, 1 H); 2.12 (m, 2 H); 2.03-1.65 (m, 7 H); 1.46 |
| | | (m, 1 H). |
| (18x) | 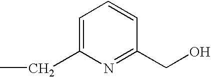 | MS (ESI pos) 443.14 (MH+)¹H-NMR (CDCl3 + D2O + Na2CO3): 7.63 (m, 1 H); 7.40-7.20 (m, 11 H); 7.09 (m, 1 H); 4.72 (s, 2 H); 3.95 (s, 2 H);3.87 (m, 1 H); 3.67 (s, 2 H); 2.96 (m, 2 H); 2.22 (m, 2 H);1.85-1.70 (m, 4 H). |
| (18y) | —CH₂COOH | MS (ESI pos) 380.2 (MH+) |
| | | ¹H-NMR (DMSO): 7.96 (s, 1 H); 7.40-7.29 (m, 8 H); 7.23 |
| | | (m, 2 H); 3.91 (s, 2 H); 3.55 (m, 1 H); 3.11 (s, 2 H); 3.03 |
| | | (m, 2 H); 2.39 (m, 2 H); 1.75 (m, 2 H); 1.52 (m, 2 H). |

| Starting | | | |
|---|---|---|---|
| material | Product | structure | analytical |
| (17c) | (l8ab) | 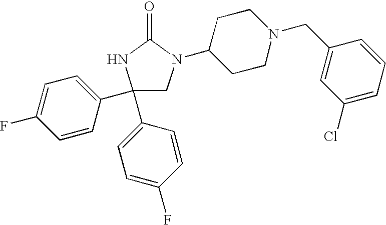 | MS (ESI POS): 482.14 (MH+)1 H NMR (CDCl3 + D2O +Na2CO3): 7.32 (s br, 1 H); 7.24-7.12 (m,7 H); 7.02 (dd, 4 H); 3.89 (s, 2 H);3.84 (m, 1 H); 3.46 (s, 2 H); 2.91 (m, 2 H);2.11 (m, 2 H); 1.77-1.68 (m, 4 H). |
| (17a) | (18ac) | 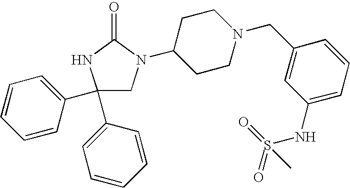 | MS (ESI POS): 519.22 (MH+)1 H NMR (1 H CDCl3): 7.39-7.18 (m,14 H); 4.91 (s br, 1 H); 3.95 (s, 2 H);3.85 (m, 1 H); 3.50 (s, 2 H); 3.32 (s,3 H); 2.91 (m, 2 H); 2.84 (s, 3 H);2.10 (m, 2 H); 1.74 (m, 4 H). |
| (17b′) | (18ad) | 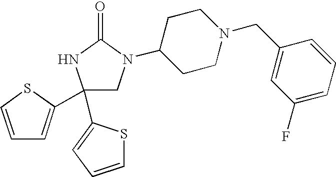 | MS (ESI POS): 441.96 (MH+)1 H NMR (CDCl3 − D2O −Na2CO3): 7.30-7.19 (m, 3 H); 7.08-6.88 (m, 7 H); 3.94 (s, 2 H); 3.86 (m,1 H); 3.48 (s, 2 H); 2.90 (m, 2 H);2.09 (ddd, 2 H); 1.76-1.62 (m, 4 H). |
| (17d) | (18ae) | 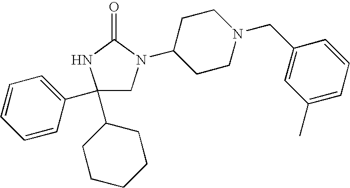 | MS (ESI POS): 432.22 (MH+)1 H NMR (CDCl3 + D2O + Na2CO3):7.34 (m, 2 H); 7.28-7.15 (m, 5 H);7.07 (m, 2 H); 3.76 (m, 1 H); 3.73 (d, 1 H);3.45 (d, 1 H); 3.43 (s, 2 H); 2.90 (m, 2 H);2.33 (s, 3 H); 2.04 (m, 2 H); 1.86-l.51 (m,9 H); 1.31-0.99 (m, 5 H); 0.75 (m, 1 H). |
| (17d) | (18af) | 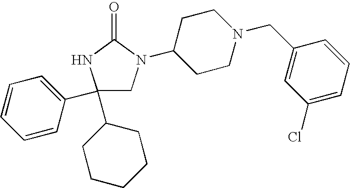 | MS (ESI POS): 452.15 (MH+)1 H NMR (1 H CDCl3 + D2O +Na2CO3): 7.38-7.12 (m, 9 H);3.79 (m, 1 H); 3.74 (d, 1 H); 3.46 (d,1 H); 3.44 (s, 2 H); 2.87 (m, 2 H);2.06 (m, 2 H); 1.86-1.50 (m, 9 H);1.31-0.99 (m, 5 H); 0.75 (m, 1 H). |
| (17b′) | (18ag) | 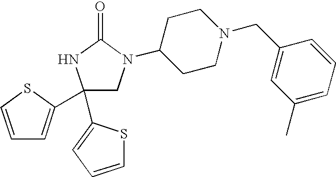 | MS (ESI POS): 438.02 (MH+)1 H NMR (CDCl3 + D2O + Na2CO3):7.19 (m, 2 H); 7.07 (m, 4 H); 6.96 (m,4 H); 3.94 (s, 2 H); 3.85 (m, 1 H);3.45 (s, 2 H); 2.93 (m, 2 H); 2.34 (s,3 H); 2.08 (m, 2 H); 1.72 (m, 4 H). |
| (17a′) | (18aj) | 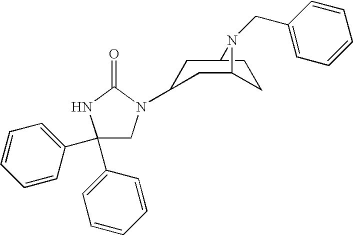 | MS (ESI POS): 438.08 (MH+)1 H NMR (CDCl3 + D2O +Na2CO3 mix of diast.): 7.41-7.20 (m, 15 H); 4.40 and 4.22 (m,1 H); 3.95 and 3.87 (s, 2 H); 3.53and 3.41 (s, 2 H); 3.25 (m, 2 H);2.32 and 2.09 (m, 1 H); 2.05 (m,1 H); 1.90-1.72 (m, 3 H); 1.63-1.45 (m, 2 H); 1.33 (m, 1 H). |
| (17a) | (18ak) | 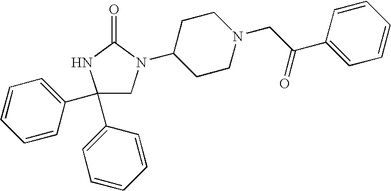 | MS (ESI POS): 440.01 (MH+)1 H NMR (1 H CDCl3 + D2O +Na2CO3 328 K): 7.98 (d, 2 H);7.55 (dd, 1 H); 7.44 (dd, 2 H); 7.38-7.22 (m, 10 H); 3.96 (s, 2 H); 3.90 (m,1 H); 3.80 (s, 2 H); 3.07 (m, 2 H);2.35 (ddd, 2 H); 1.94-1.71 (m, 4 H). |
| (17b) | (18al) | 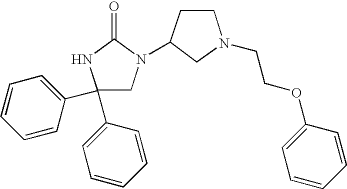 | MS (ESI POS): 428.2 (MH+)1 H NMR ‘(1 H CDCl3 + D2O +Na2CO3): 7.38-7.18 (m, 12 H);6.93 (dd, 1 H); 6.86 (d, 2 H);4.65 (m, 1 H); 4.10 and3.98 (ABq, 2 H); 4.07 (dd, 2 H);3.03-2.74 (m, 4 H); 2.60 (dd,1 H); 2.38 (dt, 1 H); 2.15 (m, 1 H);1.80 (m, 1 H). |
| (17c) | (18an) | 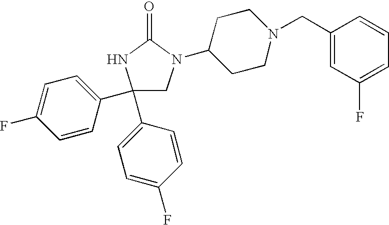 | MS (ESI POS): 466.3 (MH+)1 H NMR (1 H CDCl3 + D2O +Na2CO3): 7.24-7.15 (m, 5 H);7.08-6.97 (m, 6 H); 6.93 (m, 1 H);3.89 (s, 2 H); 3.84 (m, 1 H); 3.48 (s,2 H); 2.92 (m, 2 H); 2.10 (m, 2 H);1.78-1.67 (m, 4 H). |

### Procedure 12

### Final Products (18) Described in Scheme 5

### Synthesis of 1-[1-(4-chloro-benzyl)-piperidin-4-yl]-4,4-diphenyl-imidazolidin-2-one (18q)

Compound (17a) (0.100 g, 0.311 mmoles) is dissolved in methanol (10 mL) under nitrogen atmosphere and 4-chloro benzaldehyde (0.050 g, 0.341 mmoles) is added. The mixture is stirred at room temperature for 30 minutes, then solid NaBH₃CN (0.040 g, 0.622 mmoles) is added. The reaction is stirred at room temperature for 18 hours, then the solvent is evaporated under vacuum. The residue is dissolved in water and extracted with ethyl acetate; the organic phase is washed with water, then with brine, finally dried and concentrated under vacuum to give a yellowish solid which is purified by flash chromatography (SiO₂, 6 g; eluent: DCM:MeOH=9:1) to give the desired product as a white solid (0.074 g).

Compounds described in Table 11 were synthesized following the same procedure, starting from (17a) by reaction with the corresponding aldehydes.

TABLE 11

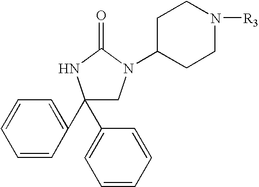

| Product | R3 | analytical |
|---|---|---|
| (18a′) | 3-OH benzyl | MS (ESI pos) 428.15 (MH+) |
| | | ¹H-NMR (CDCl3 + D2O + Na2CO3): 7.41-7.20 (m, |
| | | 10 H); 7.16 (dd, 1 H); 6.83 (d, 1 H); 6.82 (d, 1 H); |
| | | 6.71 (dd, 1 H); 3.95 (s, 2 H); 3.84 (m, 1 H); 3.44 (s, 2 H); |
| | | 2.93 (m, 2 H); 2.09 (m, 2 H); 1.79-1.69 (m, 4 H). |
| (18b′) | 2-thienyl methyl | MS (ESI pos) 418.03 (MH+) |
| | | ¹H-NMR (CDCl3 + D2O + Na2CO3): 7.38-7.17 (m, |
| | | 11 H); 6.91 (m, 2 H); 3.95 (s, 2 H); 3.84 (m, 1 H); 3.71 (s, |
| | | 2 H); 2.98 (m, 2 H); 2.12(m, 2 H); 1.79-1.68 (m, 4 H). |
| (18c′) | 2-furyl methyl | MS (ESI pos) 402.18 (MH+) |
| | | ¹H-NMR (CDCl3 + D2O + Na2CO3): 7.38-7.20 (m, 11 H); |
| | | 6.30 (m, 1 H); 6.19 (m, 1 H); 3.92 (s, 2 H); 3.84 (m, 1 H); |
| | | 3.53 (s, 2 H); 2.96 (m, 2 H); 2.12 (m, 2 H); 1.81-1.70 |
| | | (m, 4 H). |
| (18d′) | 4-Cl benzyl | MS (ESI pos) |
| | | ¹H-NMR (CDCl3 + D2O + Na2CO3): 7.37-7.20 (m, |
| | | 14 H); 3.95 (s, 2 H); 3.85 (m, 1 H); 3.44 (s, 2 H); 2.90 (m, |
| | | 2 H); 2.08 (m, 2 H); 1.78-1.68 (m, 4 H). |
| (18e′) | 3-OMe benzyl | MS (ESI pos) 442.22 (MH+) |
| | | ¹H-NMR (CDCl3): 7.37-7.17 (m, 11 H); 6.88 |
| | | (m, 2 H); 6.78 (dd, 1 H); 4.89 (s br, 1 H); |
| | | 3.95 (s, 2 H); 3.86 (m, 1 H); 3.80 |
| | | (s, 3H); 3.47 (s br, 2 H); 2.94 (m, 2 H); 2.09 (m, 2 H); |
| | | 1.80-1.68 (m, 4 H). |
| (18f′) | 4-OMe benzyl | MS (ESI pos) |
| | | ¹H-NMR |
| (18g′) | 4-OCF₃ benzyl | MS (ESI pos) MH+ 496.3 |
| | | ¹H-NMR (CDCl3): 7.37-7.21 (m, 12 H); 7.14 (d br, 2 H), |
| | | 4.88 (s br, 1 H); 3.95 (s, 2 H); 3.87 (m, 1 H); 3.48 (s br, |
| | | 2 H); 2.91 (d br, 2 H), 2.1 (m, 2 H); 1.80-1.69 (m, 4 H). |
| (18h′) | 3-COOH benzyl | MS (ESI pos) 456.10 (MH+) |
| | | ¹H-NMR (CD3OD + Na2CO3): 7.89 (s br, 1 H); 7.84 (m, |
| | | 1 H); 7.39-7.19 (m, 12 H); 3.99 (s, 2 H); 3.71 |
| | | (tt, 1 H); 3.55 (s, 2 H); 3.98 (m, 2 H); |
| | | 2.11 (ddd, 2 H); 1.78 (dq, 2 H); |
| | | 1.63 (m, 2 H). |
| (18i′) | 4-benzyloxy benzyl | MS (ESI pos) 518.29 (MH+) |
| | | ¹H-NMR (CDCl3 + D2O + Na2CO3): 7.45-7.16 (m, 17 H); |
| | | 6.91 (d, 2 H); 5.05 (s, 2 H); 3.94 (s, 2 H); |
| | | 3.85 (m, 1 H); 3.42 (s, 2 H); 2.92 (m, 2 H); 2.06 (m, |
| | | 2 H); 1.77-1.67 (m, 4 H). |
| (18j′) | 3-benzyloxy benzyl | MS (ESI pos) 518.17 (MH+) |
| | | ¹H-NMR (CDCl3 + D2O + Na2CO3): 7.45-7.16 (m, 16 H); |
| | | 6.96 (m, 1 H); 6.87 (m, 2 H); 5.06 (s, 2 H); 3.95 (s, 2 H); |
| | | 3.84 (m, 1 H); 3.46 (s, 2 H); 2.91 (m, 2 H); 2.07 |
| | | (m, 2 H); 1.76-1.66 (m, 4 H). |
| (18k′) | 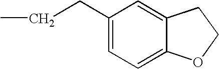 | MS (ESI pos) 454.31 (MH+) |
| (18l′) | 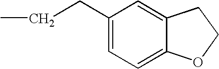 | MS (ESI pos) 456.16 (MH+)¹H-NMR (CDCl3 + D2O +Na2CO3): 7.40-7.20 (m, 11 H);6.82 (s, 1 H); 6.72 (s, 2 H);5.92 (s, 2 H); 3.94 (s, 2 H); 3.84(m, 1 H); 3.39 (s, 2 H); 2.91(m, 2 H); 2.05 (m, 2 H); 1.80-1.64 (m, 4 H). |
| (18m′) | 3-pyridyl methyl | MS (ESI pos) 413.19 (MH+) |
| | | ¹H-NMR (CDCl3 + D2O + Na2CO3): 8.53 (d br, 1 H); |
| | | 8.49 (dd br, 1 H); 7.63 (m, 1 H); 7.37-7.20 (m, 11 H); |
| | | 3.95 (s, 2H); 3.86 (m, 1 H); 3.50 (s, 2 H); 2.91 (m, 2 H); |
| | | 2.13 (m, 2 H); 1.80-1.68 (m, 4 H). |
| (18n′) | phenylethyl | MS (ESI pos) 426.18 (MH+) |
| | | ¹H-NMR (CDCl3 + D2O + Na2CO3): 7.38-7.15 (m, 15 H); |
| | | 3.95 (s, 2 H); 3.87 (m, 1 H); 3.06 (m, 2 H); 2.79 (m, 2 H); |
| | | 2.60 (m,2 H); 2.14 (m, 2 H); 1.81-1.71 (m, 4 H). |
| (18o′) | 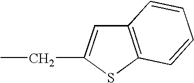 | MS (ESI pos) 468.06 (MH+)¹H-NMR (CDCl3 + D2O + Na2CO3): 7.77 (d, 1 H);7.68 (d, 1 H); 7.38-7.20 (m, 12 H); 7.13(s, 1 H); 3.97 (s, 2 H); 3.87 (m,1 H); 3.78 (s, 2 H); 3.04 (m, 2 H); 2.18 (m, 2 H);1.83-1.70 (m, 4 H). |
| (18p′) | 3,5-dichlorobenzyl | MS (ESI pos) 480.10 (MH+) |
| | | ¹H-NMR (CDCl3 + D2O + Na2CO3): 7.37-7.18 (m, 13 H); |
| | | 3.97 (s, 2 H); 3.86 (m, 1 H); 3.43 (s, 2 H); 2.88 |
| | | (m, 2 H); 2.11 (m, 2 H); 1.79-1.69 (m, 4 H). |
| (18q′) | 3-OCF₃ benzyl | MS (ESI pos) 496.14 (MH+) |
| | | ¹H-NMR (CDCl3 + D2O + Na2CO3): 7.38-7.18 (m, 13 H); |
| | | 7.09 (d br, 1 H); 3.96 (s, 2 H); 3.86 (m, 1 H); 3.50 (s, 2 H); |
| | | 2.90 (m, 2 H); 2.11 (m, 2 H); 1.80-1.69 (m, 4 H). |
| (18r′) | 3-CN benzyl | MS (ESI pos) 437.16 (MH+) |
| | | ¹H-NMR (DMSO): 7.94 (s, 1 H); 7.72 |
| | | (s, 1 H); 7.71 (d, 1 H); |
| | | 7.63 (d, 1 H); 7.53 (dd, 1 H); 7.40-7.27 (m, 8 H); 7.21 (m, |
| | | 2 H); 3.92 (s, 2 H); 3.51 (s, 2 H); |
| | | 3.50 (m, 1 H); 2.80 (m, 2 H); |
| | | 2.01 (m, 2 H); 1.67 (m, 2 H); 1.48 (m, 2 H). |
| (18s′) | 3-phenoxy benzyl | MS (ESI pos) 504.16 (MH+) |
| | | ¹H-NMR (CDCl3 + D2O + Na2CO3): 7.39-7.20 (m, 13 H); |
| | | 7.11-6.96 (m, 5 H); 6.87 (d br, 1 H); 3.94 (s, 2 H); 3.85 (m, |
| | | 1 H); 3.47 (s, 2 H); 2.92 (m, 2 H); 2.09 (m, 2 H); 1.78-1.66 |
| | | (m, 4 H). |
| (18t′) | 3-NH₂ benzyl | MS (ESI pos): 427.17 (MH+) |
| | | ¹H-NMR (CDCl3 + D2O + Na2CO3): 7.33-718 (m, 10 H); |
| | | 7.04 (dd, 1 H); 6.64 (d, 1 H); 6.63 (d, 1 H); 6.54 (dd, 1 H); |
| | | 3.91 (s, 2 H); 3.81 (m, 1 H); 3.37 (s, 2 H); 2.91 (m, 2 H); |
| | | 2.01 (m, 2 H); 1.77-1.65 (m, 4 H). |
| (18u′) | 3-thienyl methyl | MS (ESI pos): 418.15 (MH+) |
| | | ¹H-NMR (CDCl3 + D2O + Na2CO3): 7.37-7.20 (m, 11 H); |
| | | 7.10 (m, 1 H); 7.03 (m, 1 H); 3.94 (s, 2 H); 3.84 (m, 1 H); |
| | | 3.52 (s, 2 H); 2.95 (m, 2 H); 2.08 (m, 2 H); 1.79-1.68 |
| | | (m, 4 H). |

| Starting | | | |
|---|---|---|---|
| material | Product | structure | analytical |
| (17a) | (18v′) | 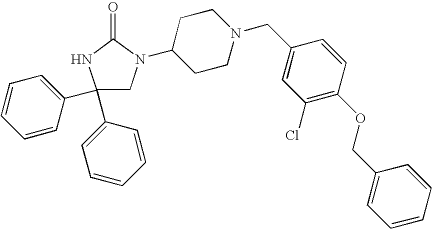 | MS (ESI POS): 552.1 (MH+)¹H NMR (CDCl3 + D2O + Na2CO3); 7.49-7.21 (m, 16 H); 7.07 (dd, 1 H); 6.89 (d, 1 H);5.13 (s, 2 H); 3.95 (s, 2 H); 3.85 (m, 1 H);3.39 (s, 2 H); 2.90 (m, 2 H); 2.06 (m, 2 H);1.77-1.67 (m 4 H). |
| (17a) | (18z′) | 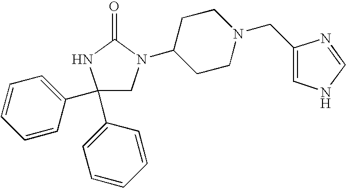 | MS (ESI POS): 402.12 (MH+)¹H NMR (CDCl3 + D2O + Na2CO3): 7.58 (sbr, 1 H); 7.37-7.20 (m, 10 H); 6.91 (s br, 1 H);3.93 (s, 2 H); 3.84 (m, 1 H); 3.52 (s, 2 H);2.98 (m, 2 H); 2.12 (m, 2 H); 1.75 (m, 4 H). |

### Procedure 13

### Final Products (19) Described in Scheme 5

### Synthesis of 1-benzyl-1-methyl-4-(2-oxo-4,4-diphenyl-imidazolidin-1-yl)-piperidinium iodide (19)

Compound (27a) (0.040 g, 0.097 mmoles) is dissolved in dichloromethane (2 ml).

Methyl iodide (0.2 ml, 3.212 mmoles) is then added and the reaction mixture is stirred for three hours at room temperature.

The reaction mixture is evaporated to afford the pure product as a white solid (0.035 g).

Compounds described in Table 12 and Table 12 bis were synthesized following the same procedure, starting from the corresponding tertiary amino derivatives and from the appropriate alkylating agent.

TABLE 12

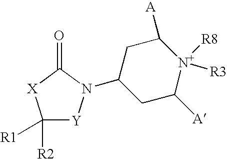

| Star- | | | | | | | | | | |
|---|---|---|---|---|---|---|---|---|---|---|
| ting | | | | | | | | | | |
| mate- | Pro- | | | | | | | | | |
| rial | duct | R1 | R2 | X | Y | R3 | R8 | A | A′ | analytical |
| (24I) | (19a) | phenyl | phenyl | NH | C═O | benzyl | methyl | —(CH2)2— | | LC-MS: 466.16 (M+) |
| | | | | | | | | | | ¹H-NMR (CDCl3): 7.60-7.30 (m, 16 H); 4.83 |
| | | | | | | | | | | (s, 2 H); 4.63 (tt, 1 H); 4.46 (m, 2 H); 3.35 (s, |
| | | | | | | | | | | 3 H); 2.88 (m, 2 H); 2.73-2.43 (m, 4 H); |
| | | | | | | | | | | 1.97 (dd, 2 H). |
| (27q) | (19b) | phenyl | phenyl | NH | CH2 | benzyl | methyl | —(CH2)2— | | LC-MS; 452.22 (M+) |
| | | | | | | | | | | ¹H-NMR (CDCl3): 7.51-7.25 (m, 15 H); 6.15 |
| | | | | | | | | | | (s br, 1 H); 4.74 (s, 2 H); 4.26 (m, 2 H); |
| | | | | | | | | | | 3.97 (s, 2 H); 3.83 (t br, 1 H); 3.24 |
| | | | | | | | | | | (s, 3 H); 2.74 (d br, 2 H); 2.54- |
| | | | | | | | | | | 2.31 (m, 4 H); 2.17 (m, 2 H). |
| (24a) | (19c) | phenyl | phenyl | NH | C═O | benzyl | methyl | H | H | LC-MS: 440.14 (M+) |
| | | | | | | | | | | ¹H-NMR (CDCl3 − 333 K): 7.72-7.30 |
| | | | | | | | | | | (m, 15 H); 6.72 (s br, 1 H); 5.10 (s, 2 H); |
| | | | | | | | | | | 4.41 (m, 1 H); 3.82 (m, 4 H); 3.34 (s, |
| | | | | | | | | | | 3 H); 2.90 (m, 2 H); 1.97 (m, 2 H). |
| (27a) | (19d) | phenyl | phenyl | NH | CH2 | benzyl | methyl | H | H | LC-MS: 426.18 (M+) |
| | | | | | | | | | | ¹H-NMR (CDCl3): 7.66-7.21 (m, 15 H); |
| | | | | | | | | | | 5.00 (s br, 1 H); 4.13 (m, 2 H); 4.09 (s, |
| | | | | | | | | | | 2 H); 3.92 (m, 1 H); 3.56 (m, 2 H); 3.23 |
| | | | | | | | | | | (s, 3 H); 2.59 (m, 2 H); 2.00 (m, 2 H). |
| (30a) | (19c) | phenyl | phenyl | N-methyl | CH2 | benzyl | methyl | H | H | LC-MS: 440.14 (M+) |
| | | | | | | | | | | ¹H-NMR (CDCl3): 7.65-7.18 (m, 15 H); |
| | | | | | | | | | | 4.97 (s, 2 H); 4.21 (m, 2 H); 3.91 (m, 1 H); |
| | | | | | | | | | | 3.90 (s, 2 H); 3.54 (m, 2 H); 3.20 (s, 3 H); |
| | | | | | | | | | | 2.62 (m, 2 H); 2.54 (s, 3 H); 2.06 (m, 2 H). |
| (27l) | (19f) | phenyl | phenyl | NH | CH2 | methyl | methyl | —(CH2)2— | | MS (ESI pos): 376.2 (M+.) |
| | | | | | | | | | | ¹H-NMR (CDCl3): 7.47-7.20 (m, 10 H); 5.40 |
| | | | | | | | | | | (s, 1 H); 4.05 (s, 2 H); 3.90-3.79 (m, 1 H); |
| | | | | | | | | | | 3.42 (s, 3 H); 3.25 (s, 3 H); 2.92-2.79 (m, |
| | | | | | | | | | | 2 H); 2.69- 2.43 (m, 2 H); 2.25-2.05 (m, |
| | | | | | | | | | | 4 H); 1.60-1.38 (m, 2 H) |
| (27b) | (19g) | phenyl | phenyl | NH | CH2 | methyl | methyl | H | H | MS (ESI pos): 350.32 (M+) |
| | | | | | | | | | | ¹H-NMR (CDCl3 − 323 K): 7.33-7.16 (m, |
| | | | | | | | | | | 10 H); 5.77 (s, 1 H); 4.03 (s, 2 H); 3.97-3.79 |
| | | | | | | | | | | (m, 3 H); 3.68-3.55 (m, 2 H); 3.38 (s, 3 H); |
| | | | | | | | | | | 3.30 (s, 3 H); 2.60-2.42 (m, 2 H); 2.04-1.80 |
| | | | | | | | | | | (m, 2 H). |
| (18v) | (19h) | phenyl | phenyl | NH | CH2 | methyl | (2-thienyl) | H | H | MS (ESI pos): 446.3 (M+) |
| | | | | | | | ethyl | | | ¹H-NMR (CDCl3 + D2O + Na2CO3): |
| | | | | | | | | | | 7.42-7.16 (m, 11 H); 7.01 (m, 2 H); 4.27 |
| | | | | | | | | | | (m, 2 H); 4.09 (s, 2 H); 3.94 (m, 1 H); 3.82 |
| | | | | | | | | | | (m, 2 H); 3.52 (m, 2 H); 3.36 (s, 3 H); 3.28 |
| | | | | | | | | | | (m, 2 H); 2.73 (m, 2 H); 2.07 (m, 2 H). |
| (18c) | (19i) | phenyl | phenyl | NH | CH2 | methyl | cyclohexyl- | H | H | MS (ESI pos): 432.3 (M+) |
| | | | | | | | methyl | | | |
| (27b) | (19j) | phenyl | phenyl | NH | CH2 | methyl | cyclopropyl- | H | H | MS (ESI pos): 390.2 (M+) |
| | | | | | | | methyl | | | ¹H-NMR (DMSO): 8.06 (s, 1 H); 7.39-7.31 (m, |
| | | | | | | | | | | 10 H); 3.98 (s, 2 H); 3.85 (m, 1 H); 3.63-3.18 |
| | | | | | | | | | | (m, 6 H); 3.09 (s, 3 H); 2.09 (m, 2 H); 1.71 (m, |
| | | | | | | | | | | 2 H); 1.11 (m, 1 H); 0.71 (m, 2 H); 0.43 (m, |
| | | | | | | | | | | 2 H). |
| (27b) | (19k) | phenyl | phenyl | NH | CH2 | methyl | 2-(N,N- | H | H | MS (ESI pos): 407.18 (M+) |
| | | | | | | | dimethyl | | | ¹H-NMR (DMSO): 7.95 (s, |
| | | | | | | | amino) ethyl | | | 1 H); 7.39-7.29 (m, 8 H); 7.23 (m, 2 H); |
| | | | | | | | | | | 3.90 (s, 2 H); 3.52 (tt, 1 H); 3.42 (dd, 2 H); |
| | | | | | | | | | | 3.10 (s, 9 H); 2.30 (m, 2 H); 2.70 (m, |
| | | | | | | | | | | 2 H); 2.00 (dd, 2 H); 1.70-1.47 (m, 4 H). |
| (18w) | (19l) | phenyl | phenyl | NH | CH2 | methyl | (2-tetra- | H | H | MS (ESI pos): 420.2 (M+) |
| | | | | | | | hydrofuryl) | | | ¹H-NMR (DMSO): 8.14 and 8.11 (s, 1 H); |
| | | | | | | | methyl | | | 7.42-7.18 (m, 10 H); 4.39 and 4.31 (m, 1 H); |
| | | | | | | | | | | 4.06-3.36 (m, 11 H); 3.13 (s, 3 H); |
| | | | | | | | | | | 2.25-1.99 (m, 3 H), 1.94-1.63 (m, 4 H); |
| | | | | | | | | | | 1.62-1.39 (m, 1 H). |

| | | | | | | | | | |
|---|---|---|---|---|---|---|---|---|---|
| (17a) | (19m) | phenyl | phenyl | NH | CH2 | —(CH2)4— | H | H | MS (ESI pos): 376.15 (M+) |
| | | | | | | | | | ¹H-NMR (DMSO): 8.08 (s, 1 H); 7.40- |
| | | | | | | | | | 7.20 (m, 10 H); 3.98 (s, 2 H); 3.87 (m, |
| | | | | | | | | | 1 H); 3.57 (m, 2 H); 3.46 (m, 4 H); 3.38 |
| | | | | | | | | | (m, 2 H); 2.05 (m, 4 H); 1.74 (m, 2 H); |
| | | | | | | | | | 1.43 (m, 2 H). |
| (17a) | (19n) | phenyl | phenyl | NH | CH2 | 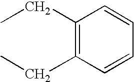 | H | H | MS (ESI pos): 424.14 (M+)¹H-NMR (DMSO): 8.12 (s, 1 H); 7.49-7.22 (m, 14 H); 5.01 (s, 2 H); 4.89 (s,2 H); 4.02 (s, 2 H); 3.95 (m, 1 H); 3.77-3.59 (m, 4 H); 2.20 (m, 2 H); 1.81 (m,2 H). |
| (17a) | (19o) | phenyl | phenyl | NH | CH2 | —(CH2)2—O—(CH2)2— | H | H | MS (ESI pos): 392.17 (M+) |
| | | | | | | | | | ¹H-NMR (DMSO): 8.08 (s, 1 H); 7.38- |
| | | | | | | | | | 7.21 (m, 10 H); 3.98 (s, 2 H); 3.98-3.80 |
| | | | | | | | | | (m, 7 H); 3.62 (m, 2 H); 3.46-3.25 (m, |
| | | | | | | | | | 4 H); 2.13 (m, 2 H); 1.71 (m, 2 H). |

| | | | | | | | | | |
|---|---|---|---|---|---|---|---|---|---|
| (27p) | (19p) | phenyl | phenyl | NH | CH2 | methyl | methyl | 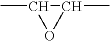 | MS (ESI pos): 390.2 (M+)¹H-NMR (DMSO): 8.24 (s, 1 H);7.45 (d, 4 H); 7.33 (dd, 4 H); 7.24 (dd,2 H); 4.40 (m, 1 H); 4.29 (s, 2 H); 4.07 (d,2 H); 4.03 (s, 2 H); 3.24 (s, 3 H); 3.01 (s,3 H); 2.63 (ddd, 2 H); 1.76 (d, 2 H) |

TABLE 12
bis

| Starting | | | |
|---|---|---|---|
| material | Product | structure | analytical |
| (27o) | (19g) | 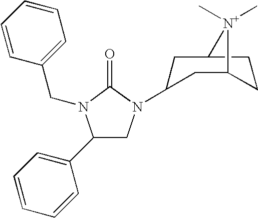 | MS (ESI POS): 390.15 (M+)1H NMR (DMSO): 7.43-7.22 (m, 8 H); 7.04 (m, 2 H); 4.62 (d, 1 H);4.39 (dd, 1 H); 4.06-3.87 (m, 3 H); 3.78 (dd, 1 H); 3.55 (d, 1 H); 3.30 (s,3 H); 3.20 (m, 1 H); 3.03 (s, 3 H); 2.61-2.25 (m, 4 H); 2.05 (m, 2 H);1.86 (m, 2 H). |
| (18d) | (19r) | 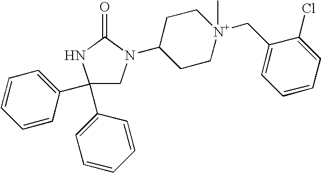 | MS (ESI POS): 460.04 (M+)1 H NMR (DMSO): 8.10 and 8.07 (s, 1 H); 7.70 (m, 1 H); 7.65 (m,1 H); 7.58 (m, 1 H); 7.51 (m, 1 H); 7.41-7.20 (m, 10 H); 4.80 and4.70 (s, 2 H); 4.08 and 3.99 (s, 2 H); 3.85 (m, 1 H); 3.70-3.43 (m, 4 H);3.08 and 2.89 (s, 3 H); 2.45-2.04 (m, 2 H); 1.89-1.69 (m, 2 H). |
| (18e) | (19s) | 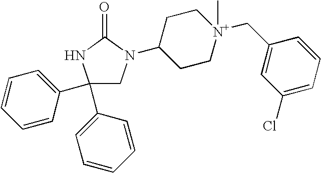 | MS (ESI POS): 460.04 (M+)1H NMR (DMSO): 8.07 (s, 1 H); 7.67-7.47 (m, 4 H); 7.41-7.20 (m,10 H); 4.67 and 4.56 (s, 2 H); 4.09 and 3.99 (s, 2 H); 3.96-3.74 (m, 1 H);3.60-3.32 (m, 4 H); 3.01 and 2.89 (s, 3 H); 2.45-2.04 (m, 2 H); 1.85-1.69 (m, 2 H). |
| (18f) | (19t) | 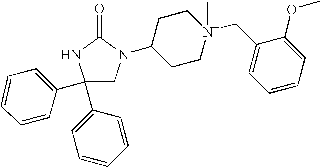 | MS (ESI POS): 456.10 (M+)1H NMR (DMSO): 8.09 and 8.06 (s, 1 H); 7.53 (dd, 1 H); 7.47 (d,1 H); 7.41-7.21 (m, 10 H); 7.19 (d, 1 H); 7.07 (dd, 1 H); 4.62 and4.51 (s, 2 H); 4.08 and 3.98 (s, 2 H); 3.85 and 3.84 (s, 3 H); 3.85-3.73 (m, 1 H); 3.59-3.33 (m, 4 H); 2.99 and 2.82 (s, 3 H); 2.50-2.02 (m, 2 H); 1.85-1.65 (m, 2 H) |
| (18g) | (19u) | 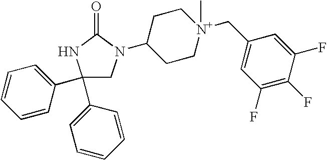 | MS (ESI POS): 480.03 (M+)1H NMR (DMSO): 8.09 and 8.08 (s, 1 H); 7.57 (m, 2 H); 7.39-7.21 (m, 10 H); 4.64 and 4.54 (s, 2 H); 4.08 and 3.99 (s, 2 H); 3.81 (m,1 H); 3.58-3.32 (m, 4 H); 3.03 and 2.92 (s, 3 H); 2.45-2.03 (m, 4 H). |
| (18m) | (19z) | 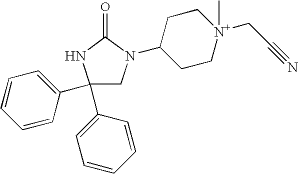 | MS (ESI POS): 375.09 (M+)1H NMR (DMSO): 8.11 and 8.10 (s, 1 H); 7.38-7.21 (m, 10 H); 5.05and 4.87 (s, 2 H); 4.00 and 3.98 (s, 2 H); 3.91-3.53 (m, 5 H); 3.27 (s,3 H); 2.18 (m, 2 H); 1.81 (m, 2 H). |
| (27q) | (19aa) | 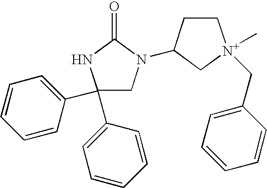 | MS (ESI POS): 412.11 (M+)1H NMR (DMSO): 8.33 (s, 1 H); 7.60-7.46 (m, 5 H); 7.41-7.20 (m, 10 H);4.81-4.61 (m, 1 H); 4,64 and 4.55 (s, 2 H); 4.09 (dd, 1 H); 3.99 (dd, 1 H);3.92-3.40 (m, 4 H); 3.04 and 2.92 (s, 3 H); 2.47-2.12 (m, 2 H). |
| (18f′) | (19ab) | 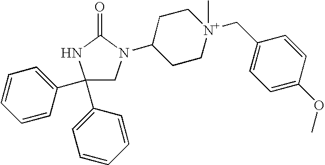 | MS (ESI POS): 456.10 (M+)1H NMR (DMSO): 8.08 (s, 1 H); 7.46 (m,2 H); 7.40-7.21 (m, 10 H); 7.06 (d,2 H); 4.60 and 4.49 (s, 2 H); 4.09 and 3.98 (s, 2 H);3.80 (s, 3 H); 3.80 (m, 1 H);3.56-3.28 (m, 4 H); 2.95 and 2.83 (s, 3 H); 2.38 and2.10 (m, 2 H); 1.75 (m, 2 H). |
| (30b) | (19ac) | 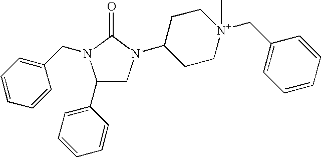 | MS (ESI POS): 440.6 (M+) |
| (26i) | (19ad) | 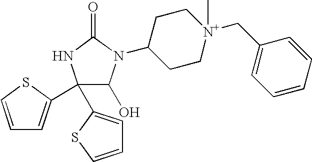 | MS (ESI POS): 453.99 (M+)1H NMR (DMSO mix of diast.): 8.29 and 8.25 (s, 1 H); 7.60-7.43 (m, 7 H); 7.16 and 7.11 (dd, 1 H); 7.06-6.92 (m, 3 H); 6.46 and6.37 (d, 1 H); 5.55 and 5.39 (d, 1 H); 4.62 and 4.55 (s, 2 H); 3.91 and3.72 (m, 1 H); 3.62-3.24 (m, 4 H); 2.96 and 2.88 (s, 3 H); 2.29 (m,2 H); 1.93 (m, 1 H); 1.77 (m, 1 H). |
| (18b′) | (19ae) | 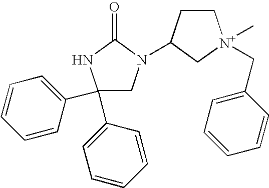 | MS (ESI POS): 432.03 (M+)1H NMR (DMSO mix of diast.): 8.08 (s, 1 H); 7.84 (m, 1 H); 7.47-7.19 (m, 12 H); 4.95 and 4.81 (s, 2 H); 4.07 and 3.99 (s, 2 H); 3.93 and3.80 (m, 1 H); 3.59-3.35 (m, 4 H); 3.03 and 2.96 (s, 3 H); 2.31 and2.13 (m, 2 H); 1.78 (m, 2 H). |
| (18ag) | (19af) | 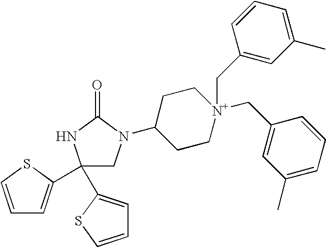 | MS (ESI POS): 542.03 (M+)1H NMR (CDCl3 + D2O + Na2CO3): 7.50 (s br, 1 H); 7.47 (m, 1 H);7.38-7.21 (m, 8 H); 6.99 (m, 4 H); 5.33 (s, 2 H); 5.77 (s, 2 H); 4.07 (s,2 H); 4.02 (m, 2 H); 3.71 (m, 1 H); 3.31 (m, 2 H); 3.58 (m, 2 H); 2.40 (s,3 H); 2.37 (s, 3 H); 1.98 (m, 2 H). |
| (18i) | (19ag) | 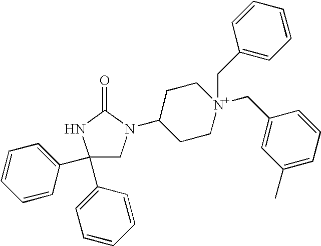 | MS (ESI POS): 530.14 (M+)1H NMR (CDCl3 + D2O + Na2CO3): 7.54-7.12 (m, 18 H); 4.75 (s,4 H); 4.09 (s, 2 H); 4.02 (d br, 2 H); 3.72 (m, 1 H); 3.31 (m, 2 H);2.59 (m, 2 H); 2.39 (s, 3 H); 2.37 (s, 3 H); 1.98 (m, 2 H); |
| (27a) | (19ah) | 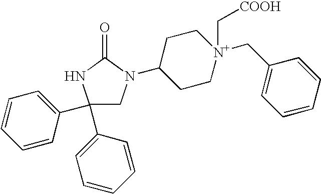 | MS (ESI POS): 470.0 (MH+)1H NMR (DMSO + TFA): 8.04 (s br, 1 H); 7.62-7.18 (m, 15 H);4.75 (s, 2 H); 4.21 (s, 2 H), 3.98 (s, 2 H); 3.90 (m, 3 H); 3.57 (m, 2 H);2.15 (m, 2 H); 1.75 (m, 2 H). |
| (30d) | (19ai) | 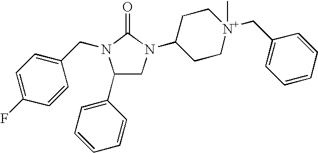 | MS (ESI POS): 458.1 (M+)1H NMR (CDCl3 + D2O + Na2CO3): 7.65 (dd, 2 H); 7.51-7.29 (m,6 H); 7.22-7.11 (m, 2 H); 7.07-6.87 (m, 4 H); 5.08 (s, 2 H); 4.31 (dd,1 H); 4.06 (m, 2 H); 3.95-3.51 (m, 6 H); 3.26 (s, 3 H); 3.18 (dd, 1 H);2.53 (m, 2 H); 2.03 (m, 2 H). |
| (30e) | (19aj) | 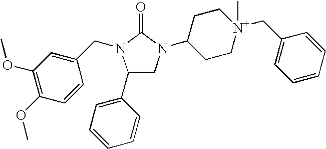 | MS (ESI POS): 500.15 (M+)1H NMR (CDCl3 + D2O + Na2CO3 mix of diast): 7.69-7.53 (m,2 H); 7.52-7.29 (m, 6 H); 7.23-7.13 (m, 2 H); 6.74 (d, 1 H); 6.56 (m,2 H); 5.02 (s, 2 H); 4.71 (d, 1 H); 4.30 (dd, 1 H); 4.05 (m, 2 H); 3.83 (s,3 H); 3.76 (s, 3 H); 3.62 (m, 3 H); 3.48 (d, 1 H); 3.23 (s, 3 H); 3.15 (dd,1 H); 2.83 (m, 1 H); 2.52 (m, 2 H); 2.04 (m, 2 H). |
| (27b) | (19ak) | 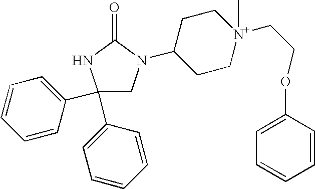 | MS (ESI POS): 456.10 (M+)1H NMR (DMSO 368 K mix of diast): 7.59 (s br, 1 H); 7.41-7.22 (m, 12 H); 7.06-6.97 (m, 3 H); 4.50 (m, 2 H); 4.03 and 4.00 (s,2 H); 3.95-3.00 (m, 7 H); 3.22 (s, 3 H); 2.35-1.77 (m, 4 H). |
| (30f) | (19al) | 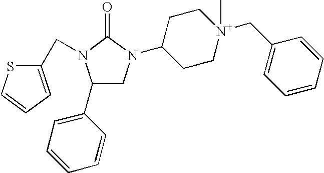 | MS (ESI POS): 446.04 (M+)1H NMR (CDCl3 + Na2CO3 + D2O mix of diast.): 7.71-7.18 (m, 11 H);6.91 (m, 1 H); 6.73 (m, 1 H); 5.04 (s, 2 H); 4.95 (m, 1 H); 4.88 (d, 1 H);4.43 (dd, 1 H); 4.24 (m, 2 H); 4.09-3.80 (m, 1 H); 3.80 (d, 1 H); 3.69 (dd,1 H); 3.58 (m, 2 H); 3.23 (s, 3 H); 2.94 and 2.63 (m, 2 H); 2.08 (m, 2 H). |
| (30g) | (19am) | 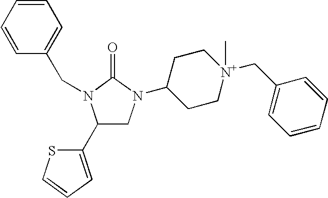 | MS (ESI POS): 446.04 (M+)1H NMR (DMSO mix of diast): 7.61-7.47 (m, 6 H); 7.38-7.21 (m, 3 H);7.16-6.97 (m, 4 H); 4.82-4.60 (m, 2 H); 4.58 (s, 2 H); 4.15-3.50 (m, 5 H); 3.49-3.32 (m, 3 H); 2.98 and 2.89 (s, 3H); 2.40and 2.14 (m, 2 H); 1.85 (m, 2 H). |
| (27z) | (19an) | 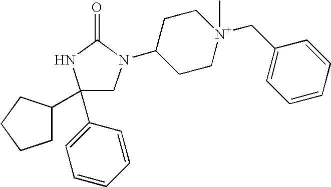 | MS (ESI POS): 418.06 (M+)1H NMR (DMSO mix of diast.): 7.58-7.16 (m, 11 H); 4.65 and4.55 (s, 2 H); 3.99-3.71 (m, 1 H); 3.83 and 3.71 (d, 1 H); 3.61-3.21 (m,5 H); 2.96 and 2.86 (s, 3 H); 2.45-0.99 (m, 13 H). |
| (30h) | (19ap) | 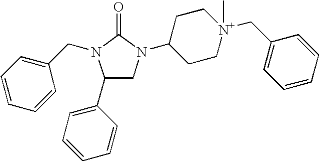 | MS (ESI POS): 440.2 (M+)1H NMR (CDCl3 + D2O + Na2CO3 mix. of diast.): 7.71-6.98 (m,15 H); 5.08 (s, 2 H); 4.98 and 4.92, 4.82-4.74 (ABq, 2 H); 4.38 and4.32 (dd, 1 H); 4.05 (m, 2 H); 3.93-3.49 (m, 5 H); 3.26 (s, 3 H); 3.26and 3.18 (dd, 1 H); 2.89 and 2.52 (m, 2 H); 2.18-1.96 (m,2 H). |
| (27p) | (19aq) | 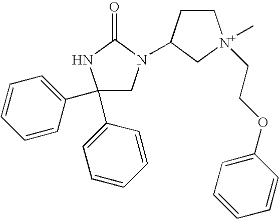 | MS (ESI POS): 442.2 (M+)1H NMR (1H CDCl3 + D2O + Na2CO3 mix. of diast.): 7.40-7.21 (m, 12 H); 7.04 (dd, 1 H); 6.90 (m, 2 H); 4.91 (m, 1 H); 4.52-4.10 (m, 10 H); 3.53 and 3.42 (s, 3 H); 2.73-2.52 (m, 2 H). |
| (27r) | (19ar) | 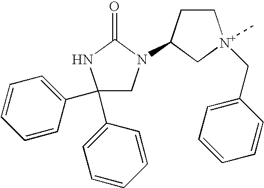 | MS (ESI POS): 412.2 (M+)1H NMR (CDCl3 + D2O + Na2CO3 mix. of diast.): 7.78-7.20 (m,15 H); 5.08-4.80 (m, 3 H); 4.56-4.14 (m, 4 H); 3.94-3.59 (m, 2 H); 3.31and 3.18 (s, 3 H); 2.72-2.44 (m, 2 H). |
| (27s) | (19as) | 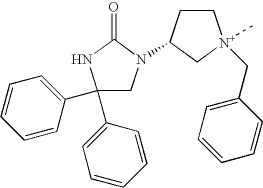 | MS (ESI POS): 412.2 (M+)1H NMR (CDCl3 +D2O + Na2CO3 mix. of diast): 7.65-7.19 (m,15 H); 5.08-4.87 (m, 3H); 4.58-4.14 (m, 4 H); 3.96-3.57 (m, 2 H); 3.31and 3.17 (s, 3 H); 2.71-2.46 (m, 2 H). |
| (27h) | (19at) | 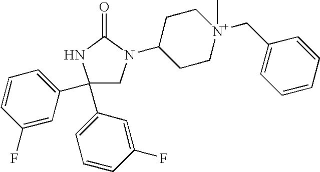 | MS (ESI POS): 462.2 (M+)1H NMR (CDCl3 + D2O + Na2CO3 mix. of diast.): 7.64-7.43 (m,6 H); 7.33 (m, 2 H); 7.12-6.92 (m, 5 H); 4.92 and 4.75 (s, 2 H); 4.23and 4.10 (s, 2 H); 4.18 (m, 2 H); 3.97 (m, 1 H); 3.68 and 3.52 (m, 2 H);3.22 (s, 3 H); 2.95 and 2.63 (m, 2 H); 2.04 (m, 2 H). |
| (27x) | (19au) | 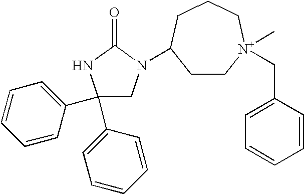 | MS (ESI POS): 440.3 (M+)1H NMR (DMSO 368 K mix of diast): 7.68(s br, 1 H); 7.61-7.46 (m, 5 H);7.43-7.20 (m, 10 H); 4.55 and 4.29 (m, 2 H);4.12-3.83 (m, 3 H); 3.67-3.22 (m,4 H); 2.97 and 2.77 (s, 3 H); 2.43-2.20(m, 2 H); 2.10-1.69 (m, 4 H). |
| (27p) | (19aw) | 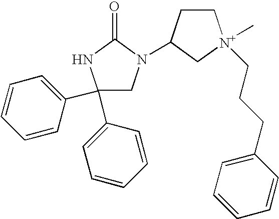 | MS (ESI POS): 440.2 (M+)1H NMR (CDCl3 + D2O + Na2CO3 328 K mix of diast.): 7.39-7.13 (m, 15 H); 4.89 (m, 1 H); 4.30-3.54 (m, 6 H); 4.21 (s, 2 H); 3.31and 3.26 (s, 3 H); 2.89-2.36 (m, 4 H); 2.11 (m, 2 H). |
| (18aI) | (19ax) | 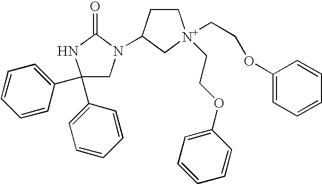 | MS (ESI POS): 548.3 (M+)1H NMR (DMSO 368 K): 7.89 (s br, 1 H); 7.41-7.23 (m, 14 H);7.05-6.94 (m, 6 H); 4.68 (m, 1 H); 4.51 (m, 4 H);4.17-3.83 (m, 10 H); 2.45 (m, 2 H). |
| (18aj) | (19ay) | 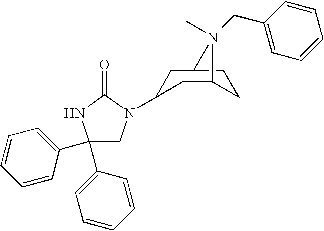 | MS (ESI POS): 452.8 (M+)1H NMR (1H DMSO mix of diast): 8.19 and 8.13 (s, 1 H); 7.60-7.44 (m, 6 H); 7.42-7.31 (m, 6 H); 7.28-7.20 (m, 3 H); 4.98 and 4.72 (s,2 H); 4.12 and 4.09 (s, 2 H); 4.02 (m, 1 H); 3.92 (m, 2 H); 2.82 (m, 2 H);2.72 and 2.62 (s, 3 H); 2.31 (m, 2 H); 2.08 (m, 2 H); 1.92 (m, 2 H). |
| (27p) | (19ba) | 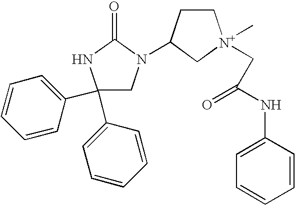 | MS (ESI POS): 455.3 (M+)1H NMR (DMSO + Na2CO3 mix of diast.): 10.53 (s br, 1 H);8.34 (s, 1 H); 7.57 (d, 2 H); 7.44-7.20 (m, 12 H); 7.13 (dd, 1 H);4.70 (m, 1 H); 4.49 and 4.40 (s, 2 H); 4.20-3.96 (m, 3 H); 3.92-3.77 (m,2 H); 3.69 (m, 1 H); 3.38 and 3.27 (s, 3 H); 2.47-2.14 (m, 2 H). |
| (27p) | (19bb) | 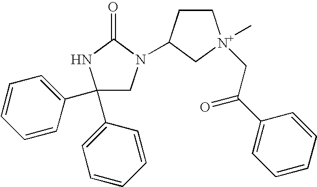 | MS (ESI POS): 440.3 (M+)1H NMR (DMSO mix of diast.): 8.33 (s, 1 H); 7.98 (m, 2 H);7.76 (m, 1 H); 7.62 (m, 2 H); 7.45-7.20 (m, 10 H); 5.44 and 5.41-5.37and 5.31 (ABq, 2 H); 4.71 (m, 1 H); 4.22-3.96 (m, 3 H); 3.86 (m, 2 H);3.74 (m, 1 H); 3.38 and 3.29 (s, 3 H); 2.48-2.19 (m, 2 H). |
| (27p) | (19bc) | 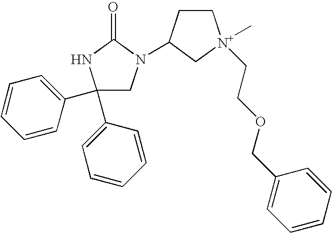 | MS (ESI POS): 456.3 (M+)1H NMR (CDCl3 mix of diast.): 7.39-7.21 (m, 15 H); 5.40 and5.37 (s, 1 H); 4.87 (m, 1 H); 4.55 (s, 2 H); 4.41-3.77 (m, 10 H); 3.42and 3.31 (s, 3 H); 2.67-2.35 (m, 2 H). |
| (27p) | (19bd) | 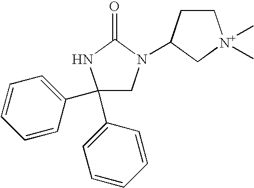 | MS (ESI POS): 336.2 (M+)1H NMR (DMSO 368 K): 7.84 (s br, 1 H); 7.43-7.22 (m, 10 H); 4.62(m, 1 H); 4.10-3.99 (ABq, 2 H); 3.86-3.56 (m, 4 H); 3.24 (s,3H); 3.16 (s, 3 H); 2.38 (m, 2 H). |
| (18ak) | (19be) | 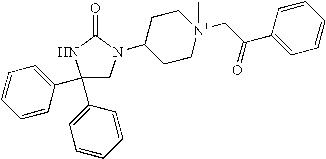 | MS (ESI POS): 454.3 (M+)1H NMR (DMSO): 8.12 (s, 1 H); 7.99 (d, 2 H); 7.76 (dd, 1 H);7.62 (dd, 2 H); 7.41-7.23 (m, 10 H); 5.28 (s, 2 H); 4.03 (s, 2 H); 3.84 (m,3 H); 3.62 (m, 2 H); 3.35 (s, 3 H); 2.25 (m, 2 H); 1.81 (m, 2 H). |
| (27s) | (19bf) | 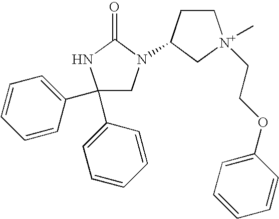 | MS (ESI POS): 442.2 (M+)1H NMR (CDCl3 mix of diast): 7.41-7.22 (m, 12 H); 7.05 (dd, 1 H);6.91 (m, 2 H); 5.27 and 5.20 (s, 1 H); 4.86 (m, 1 H); 4.55-4.00 (m, 9 H);3.89 (m, 1 H); 3.55 and 3.42 (s, 3 H); 2.62 (m, 2 H). |
| (27r) | (19bg) | 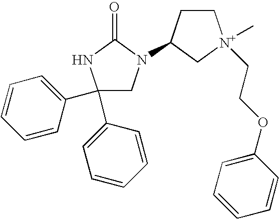 | MS (ESI POS): 442.2 (M+)1H NMR (CDCl3 mix of diast.): 7.41-7.22 (m, 12 H); 7.04 (dd, 1 H);6.91 (m, 2 H); 5.32 and 5.26 (s, 1 H); 4.88 (m, 1 H); 4.60-4.11 (m, 9 H);3.92 (m, 1 H); 3.54 and 3.42 (s, 3 H); 2.72-2.42 (m, 2 H). |
| (27ab) | (19bh) | 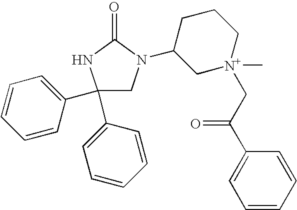 | MS (ESI POS): 454.3 (M+)1H NMR (DMSO mix of diast.): 8.42 (s, 1 H); 7.81 (dd, 2 H);7.75 (dd, 1 H); 7.56 (dd, 2 H); 7.44-7.31 (m, 9 H); 7.25 (m, 1 H); 5.26and 5.14 (Abq, 2 H); 4.16 and 4.04 (ABq, 2 H); 4.10 (m, 2 H);3.88 (dd, 1 H); 3.62 (m, 1 H); 3.49 (dd, 1 H); 3.17 (s, 3 H); 2.24-1.86 (m, 4 H). |
| (27p) | (19bi) | 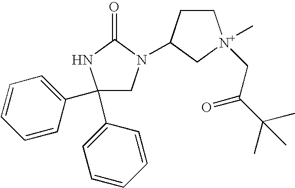 | MS (ESI POS): 420.3 (M+)1H NMR (DMSO mix of diast.): 8.35 and 8.33 (s, 1 H); 7.43-7.22 (m, 10 H); 4.96 and 4.91-4.88 and 4.82 (ABq, 2 H); 4.63 (m,1 H); 4.20-3.53 (m, 6 H); 3.23 and 3.12 (s, 3 H); 2.47-2.15 (m, 2 H);1.13 (s, 9 H). |
| (27p) | (19bj) | 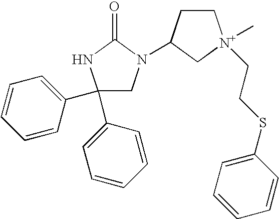 | MS (ESI POS): 458.2 (M+)1H NMR (DMSO mix of diast.): 8.34 and 8.31 (s, 1 H); 7.46-7.22 (m, 15 H); 4.79-4.59 (m, 1 H); 4.11-3.93 (m, 2 H); 3.88-3.71 (m,1 H); 3.70-3.38 (m, 7 H); 3.19 and 3.10 (s, 3 H); 2.46-2.09 (m, 2 H). |
| (27p) | (19bk) | 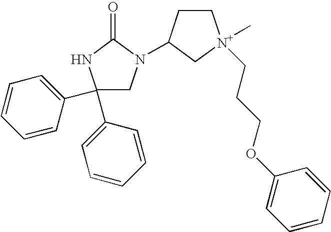 | MS (ESI POS): 456.3 (M+)1H NMR (DMSO mix of diast.): 8.34 and 8.33 (s, 1 H); 7.43-7.21 (m, 12 H); 6.95 (m, 3 H); 4.71 (m, 1 H); 4.14-3.96 (m, 4 H);3.81 (m, 11 H); 3.70-3.47 (m, 5 H); 3.17 and 3.08 (s, 3 H); 2.46-2.11 (m,4 H). |
| (27p) | (19bl) | 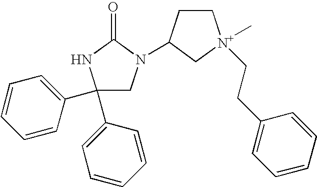 | MS (ESI POS): 426.3 (M+)1H NMR (DMSO diast.): 8.34 and 8.33 (s, 1 H); 7.44-7.21 (m, 15 H); 4.72 (m, 1 H); 4.17-3.96 (m, 2 H); 3.91 and 3.78 (dd,1 H); 3.73-3.50 (m, 5 H); 3.24 and 3.15 (s, 3 H); 3.07 (m, 2 H); 2.46-2.13 (m, 2 H). |
| (27ab) | (19bm) | 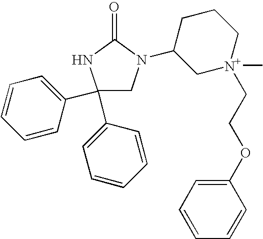 | MS (ESI POS): 456.2 (M+)1H NMR (CDCl3 mix of diast.): 8.45 (s, 1 H); 7.43-7.19 (m, 12 H);7.01 (dd, 1 H); 6.93 (d, 2 H); 4.18 (m, 2 H); 4.11 (d, 1 H); 4.00 (m, 1 H);3.93 (d, 1 H); 3.84 (m, 1 H); 3.76-3.46 (m, 3 H); 3.40-3.17 (m, 2 H);3.00 (s, 3 H); 2.19-1.84 (m, 4 H). |
| (18al) | (19bn) | 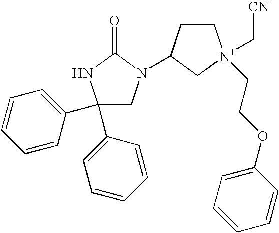 | MS (ESI POS): 467.3 (M+)1H NMR (DMSO mix of diast.): 8.38 (s, 1 H); 7.41-7.23 (m, 12 H);7.05-6.95 (m, 3 H); 5.07 and 5.01-5.00 and 4.98 (Abq, 2 H); 4.74 (m,1 H); 4.48 (m, 2 H); 4.22-3.71 (m, 8 H); 2.47-2.28 (m, 2 H). |
| (27w) | (19bo) | 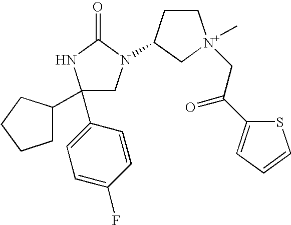 | MS (ESI POS): 438.1 (M+)1H NMR (DMSO 343 K mix of diast.): 8.58-8.49 (m, 1 H); 7.71 (m,1 H); 7.60-7.52 (m, 1 H); 7.44-7.21 (m, 5 H); 5.24 (dd, 1 H); 5.17 (m,1 H); 4.60 (m, 1 H); 4.10-3.46 (m, 5 H); 3.43 and 3.36 (s, 3 H); 3.30 (d,1 H); 2.47-2.24 (m, 2 H); 1.78-1.14 (m, 10 H). |
| (27v) | (19bp) | 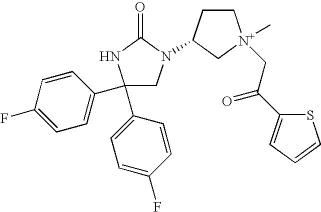 | DMSO 343 K mix of diast.): 8.16 (d, 1 H); 8.10 (s br, 1 H); 8.04 (ddd,1 H); 7.46-7.36 (m, 4 H); 7.33 (dd, 1 H); 7.21-7.10 (m, 4 H); 5.36 and5.26 (s, 2 H); 4.65 (m, 1 H); 4.16-3.73 (m, 6 H); 3.41 and 3.32 (s, 3 H);2.47-2.24 (m, 2 H). |
| (27v) | (19bq) | 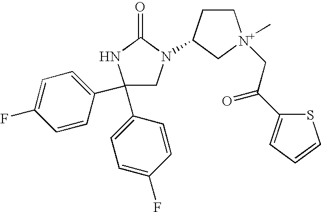 | MS (ESI POS): 482.3 (M+)1H NMR (DMSO mix of diast.): 8.60 (m, 1 H); 8.34 (s, 1 H);7.74 (m, 1 H); 7.56 (m, 1 H); 7.46-7.33 (m, 4 H); 7.25-7.12 (m, 4 H);5.32 and 5.22 (s, 2 H); 4.69 (m, 1 H); 4.18-3.93 (m, 3 H); 3.89-3.66 (m,3 H); 3.37 and 3.27 (s, 3 H); 2.47-2.17 (m, 2 H). |
| (27v) | (19br) | 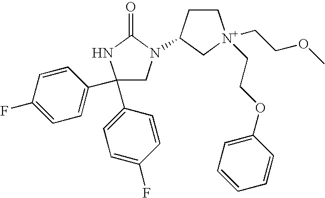 | MS (ESI POS): 522.4 (M+)1H NMR (DMSO mix of diast.): 8.35 (s, 1 H); 7.44-7.25 (m, 6 H);7.25-7.13 (m, 4 H); 7.04-6.91 (m, 3 H); 4.70 (m, 1 H); 4.40 (m, 2 H);4.12-3.74 (m, 12 H); 3.31 and 3.29 (s, 3 H); 2.45-2.15 (m, 2 H). |
| (27v) | (19bt) | 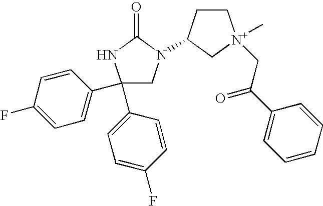 | MS (ESI POS): 476.2 (M+)1H NMR (DMSO 368 K mix of diast.): 7.97 (m, 2 H); 7.93 (s br,1 H); 7.74 (dd, 1 H); 7.60 (dd, 2 H); 7.46-7.34 (m, 4 H); 7.20-7.07 (m,4 H); 5.39-5.31 (s, 2 H); 4.64 (m, 1 H); 4.17-3.79 (m, 6 H); 3.42 and3.34 (s, 3 H); 2.48-2.29 (m, 2 H). |
| (27w) | (19bu) | 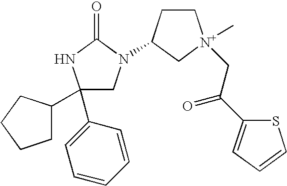 | DMSO 368 K mix of diast.): 8.12 (m, 1 H); 8.02 (m, 1 H); 7.44-7.21 (m, 7 H); 5.20 and 5.18 (s, 2 H); 4.58 (m, 1 H); 3.93-3.36 (m, 6 H);3.45 and 3.38 (s, 3 H); 2.47-2.32 (m, 2 H); 1.78-1.18 (m, 9 H). |
| (27v) | (19bv) | 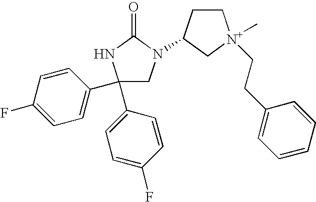 | MS (ESI POS): 462.2 (M+)1H NMR (DMSO mix of diast): 8.35 and 8.34 (s, 1 H); 7.44-7.14 (m, 13 H); 4.69 (m, 1 H); 4.11-3.48 (m, 8 H); 3.23 and 3.14 (s,3 H); 3.06 (m, 2 H); 2.46-2.14 (m, 2 H). |
| (27aa) | (19bw) | 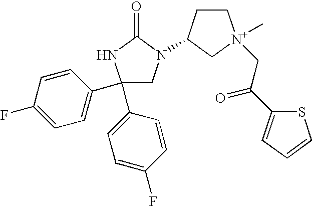 | MS (ESI POS): 482.1 (M+)1H NMR (DMSO 343 K mix of diast.): 8.16 (d, 1 H); 8.11 (s, 1 H);8.01 (m, 1 H); 7.46-7.30 (m, 5 H); 7.23-7.10 (m, 4 H); 5.29 and 5.22 (s,2 H); 4.64 (m, 1 H); 4.14-3.72 (m, 6 H); 3.39 and 3.31 (s, 3 H); 2.49-2.24 (m, 2 H). |
| (27aa) | (19bx) | 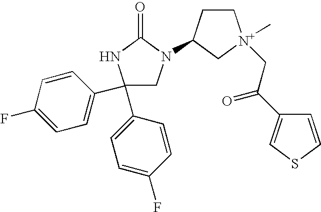 | MS (ESI POS): 482.2 (M+)1H NMR (DMSO 343 K mix of diast.): 8.54 (m, 1 H); 8.11 (s br,1 H); 7.71 (m, 1 H); 7.56 (m, 1 H); 7.46-7.33 (m, 4 H); 7.22-7.08 (m,4 H); 5.26 and 5.18 (s, 2 H); 4.65 (m, 1 H); 4.15-3.71 (m, 6 H); 3.39and 3.30 (s, 3 H); 2.45-2.24 (m, 2 H). |
| (27v) | (19by) | 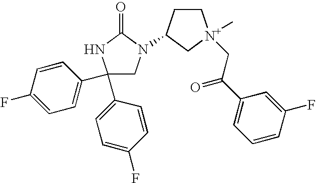 | MS (ESI POS): 494.2 (M+)1 H NMR (DMSO 343 K mix of diast.): 8.11 (s br, 1 H); 7.85-7.54 (m, 4 H); 7.46-7.33 (m, 4 H); 7.21-7.09 (m, 4 H); 5.41 and 5.37-5.33 and 5.29 (ABq, 2 H); 4.66 (m, 1 H); 4.15-3.74 (m, 6 H); 3.40 and3.32 (s, 3 H); 2.47-2.25 (m, 2 H). |
| (27v) | (19bz) | 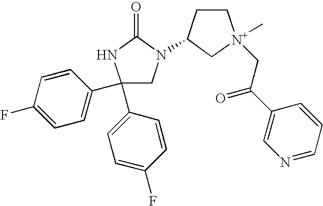 | MS (ESI POS): 477.3 (M+)1H NMR (DMSO 343 K mix of diast.): 9.13 (dd, 1 H); 8.88 (ddd,1 H); 8.29 (m, 1 H); 8.11 (s br, 1 H); 7.63 (dd, 1 H); 7.46-7.34 (m, 4 H);7.22-7.08 (m, 4 H); 5.45 and 5.40-5.37 and 5.33 (ABq, 2 H); 4.66 (m,1 H); 4.16-3.73 (m, 6 H); 3.42 and 3.33 (s, 3 H); 2.47-2.25 (m, 2 H). |
| (27v) | (19ca) | 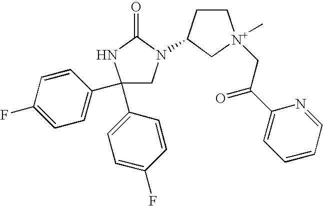 | MS (ESI POS): 477.3 (M+)1H NMR (DMSO 343 K mix of diast.): 8.75 (m, 1 H); MS (ESIPOS): 477.3 (M+)1H NMR 8.08 (m, 3 H); 7.77 (m, 1 H); 7.46-7.33 (m, 4 H); 7.22-7.09 (m, 4 H); 5.51-5.45 and 5.41 (s and ABq, 2 H); 4.75-4.56 (m,1 H); 4.17-3.76 (m, 6 H); 3.41 and 3.32 (s, 3 H); 2.46-2.24 (m, 2 H). |
| (27v) | (19cb) | 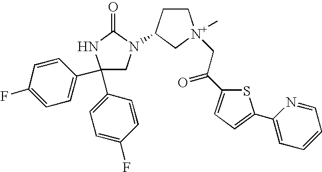 | MS (ESI POS): 559.3 (M+)1 HNMR (DMSO 343 K mix of diast.): 8.63 (m, 1 H); 8.11 (s, 1 H);8.03 (m, 2 H); 7.93 (m, 2 H); 7.46-7.33 (m, 5 H); 7.22-7.08 (m, 4 H);5.33 and 5.29-5.25 and 5.21 (ABq, 2 H); 4.67 (m, 1 H); 4.16-3.73 (m,6 H); 3.41 and 3.33 (s, 3 H); 2.48-2.26 (m, 2 H). |
| (27v) | (19cc) | 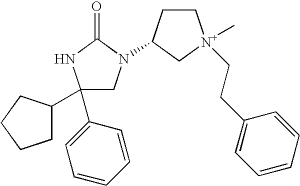 | MS (ESI POS): 418.3 (M+)1H NMR (DMSO + TFA 343 K mix of diast.): 7.45-7.20 (m, 11 H);4.58 (m, 1 H); 3.97-3.31 (m, 6 H); 3.28 and 3.22 (s, 3 H); 3.08 (m, 2 H);2.45-2.25 (m, 3 H); 1.77-1.16 (m, 10 H). |

### Procedure 13b

### Final Products (21) Described in Scheme 5

### Synthesis of 1-[1-benzyl-1-oxy)-piperidin-4-yl]-4,4-diphenyl-imidazolidin-2-one (21a)

Compound (27a) (0.100 g, 0.243 mmoles) is dissolved in dry DCM (3 ml) under nitrogen atmosphere. The reaction mixture is cooled to 0° C. and m-chloroperbenzoic acid (0.063 g, 0.364 mmoles) is added. The reaction is stirred at 0° C. for 30 minutes and then for 3 hours at room temperature.

The reaction mixture is then diluted with DCM and washed twice with a saturated solution of potassium carbonate, water and brine.

The organic phase is dried and evaporated to afford a pure product as a white solid (0.075 g).

Compound (21b) described in Table 13 was synthesized following the same procedure 13b, starting from intermediate (18ar).

TABLE 13

| Starting | | | |
|---|---|---|---|
| material | Product | | analytical |
| (27a) | (21a) | 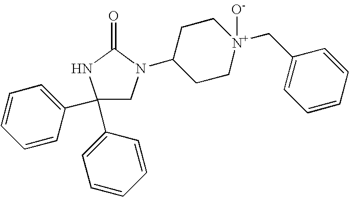 | MS (EI pos) 428.24 (M+)¹H-NMR (CDCl3): 7.53-7.38 (m,5 H); 7.36-7.19 (m, 10 H); 5.04 (s,1 H); 4.48 (s, 2 H); 4.01 (s, 2 H);3.89 (tt, 1 H); 3.34 (d br, 2 H); 3.21(ddd, 2 H); 2.65 (dddd, 2 H); 1.66 (m,2 H). |
| (18ar) | (21b) | 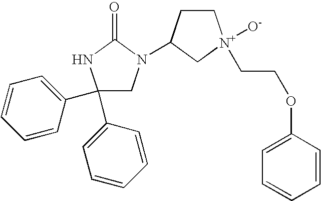 | LC-MS (ESI POS): 444.2 (MH+)NMR (DMSO, mixture ofdiastereoisomers): 8.21 and 8.03 (s,1 H); 7.43-7.14 (m, 11 H); 6.96 (m,4 H); 4.73 (m, 1 H); 4.54 (m, 2 H);4.34 (d, 1 H); 4.05 (d, 1 H); 3.80 (m,1 H); 3.72-3.51 (m, 3 H); 3.37 (m, 1 H);3.08 (m, 1 H); 2.39 (m, 1 H); 2.06 (m,1 H). |

### Procedure 14

### Final Products (44) Described in Scheme 9

### Synthesis of 1-(1-benzyl-piperidin-4-yl)-3-methyl-4-phenyl-imidazolidin-2-one (44a)

### 1. 2-(N-tertbutyloxycarbonyl)-amino-2-phenyl-N-(1-benzyl-piperidin-4-yl)-acetamide

Commercially available N—BOC protected phenyl glycine (5.0 g, 19.9 mmoles) is suspended in a mixture of acetonitrile (50 mL) and dichloromethane (50 mL). The suspension is vigorously stirred under nitrogen atmosphere. N-hydroxybenzotriazole (2.97 g, 22 mmoles) and dicyclohexylcarbodiimide (4.53 g, 22 mmoles) are added and the mixture is stirred at room temperature for 2 hours. 4-amino-N-benzylpiperidine (4.18 g, 22 mmoles) is added and the reaction is stirred at room temperature overnight.

The reaction mixture is then diluted with DCM and washed twice with a saturated solution of potassium carbonate, water and brine. The organic phase is dried and concentrated under vacuum to give an oil which is purified by flash chromatography (SiO₂, 120 g; eluent: DCM:MeOH=95:5) to give the desired product as a white solid (6.4 g).

### 2. 2-amino-N-(1-benzyl-piperidin-4-yl)2-phenylacetamide Dihydrochloride

2-(N-tertbutyloxycarbonyl)-amino-2-phenyl-N-(1-benzyl-piperidin-4-yl)-acetamide was dissolved in dichloromethane (80 mL) and added with a saturated solution of hydrogen chloride in diethyl ether (50 mL). The reaction mixture is stirred at room temperature for 4 hours. The desired product precipitates as a white solid and is then filtered and dried under vacuum (4.5 g).

### 3. N-2-(1-Benzylpiperidin-4-yl)-1-phenylethane-1,2-diamine hydrochloride

To a solution of 2-amino-N-(1-benzylpiperidin-4-yl)-2-phenylacetamide dihydrochloride (0.44 g, 1.15 mmol) in dry toluene (10 ml), borane-methylsulfide complex (0.22 ml, 2.3 mmol) was added under inert atmosphere. The mixture was refluxed for 4 hours followed by addition of MeOH (0.5 ml). After 1 h the solution was cooled and treated with 10 ml of a 10% solution of HCl in ethyl ether. The white precipitate was filtered off and washed with ether (0.45 g).

### 4. 1-(1-Benzylpiperidin-4-yl)-4-phenylimidazolidin-2-one

To a suspension of N-2-(1-benzylpiperidin-4-yl)-1-phenylethane-1,2-diamine hydrochloride (0.4 g, 0.95 mmol) in dry 1,4-dioxane (10 ml), carbonyl-diimidazole (0.2 g, 1.14 mmol) was added. The mixture was refluxed for 2 hours, and then the solvent was removed. The crude material was treated with water and extracted with ethyl acetate. The organic phase was washed with brine, dried over anidrous sodium sulphate and concentrated under reduced pressure to afford a white solid (0.22 g).

The other compounds described in Table 14 were synthesized following the same Procedure 14, starting from the corresponding commercially available starting materials.

TABLE 14

| Starting | | | |
|---|---|---|---|
| material | Product | | analytical |
| — | (44a) | 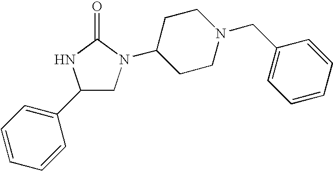 | MS (ESI pos): 360.12 (MH+) |
| — | (44b) | 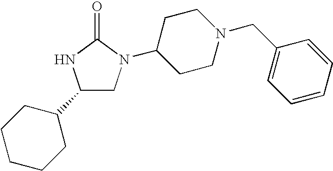 | MS (ESI POS): 342.31 (MH+) |
| — | (44c) | 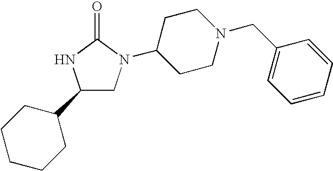 | MS (ESI POS): 342.31 (MH+) |
| — | (44d) | 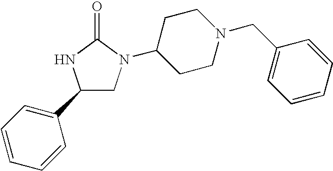 | MS (ESI POS): 336.22 (MH+) |
| — | (44e) | 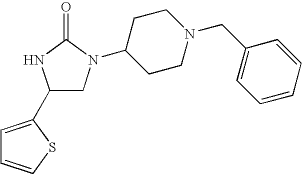 | MS (ESI POS): 342.11 (MH+) |

### Procedure 15

### Final products (30) described in Scheme 7

### Synthesis of 1-(1-benzyl-piperidin-4-yl-3-methyl-4-phenyl-imidazolidin-2-one (30a)

Compound (44a) (0.100 g, 0.243 mmoles) is dissolved in dry THF under nitrogen atmosphere: the solution is cooled to 0° C. and solid NaH (0.010 g, 0.243 mmoles) is added. The reaction is stirred at 0° C. for 15 minutes, then benzyl bromide (0.042 g, 0.243 mmoles) is added and the stirring is continued at room temperature for 3 hours. The reaction is diluted with water and extracted with ethyl acetate; the organic phase is washed with water, then with brine, finally dried and concentrated under vacuum to give an oil which is purified by preparative HPLC to give the desired product as a white solid (0.041 g).

Compounds described in Table 15 were synthesized following the same procedure, starting from the corresponding derivatives and from the appropriate alkylating agent.

TABLE 15

| Starting | | | |
|---|---|---|---|
| material | Product | Structure | analytical |
| (27a) | (30a) | 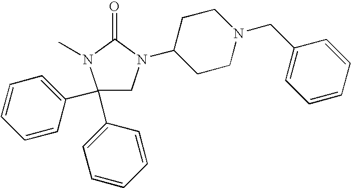 | MS (ESI pos) MH+ 426.2¹H NMR (CDCl3 − 343 K): 7.38-7.21(m, 15 H); 3.91 (m, 1 H); 3.82 (s, 2 H);3.55 (s br, 2 H); 2.97 (m, 2 H); 2.57 (s,3 H); 2.19 (m, 2 H); 1.89-1.71 (m, 4 H). |
| (44a) | (30b) | 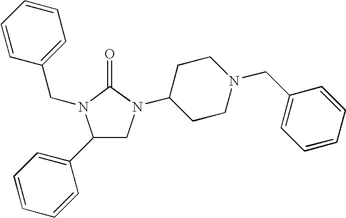 | MS (ESI pos) 426.1 (MH+)¹H NMR (CDCl3 − D2O − Na2CO3):7.39-7.06 (m, 15 H); 4.89 (d, 1 H);4.28 (dd, 1 H); 3.89 (m, 1 H); 3.59 (d,1 H); 3.56 (d, 1 H); 3.49 (s, 2 H); 3.13(dd, 1 H); 2.93 (m, 2 H); 2.11 (dt,2 H); 1.76-1.61 (m, 4 H). |
| (44a) | (30c) | 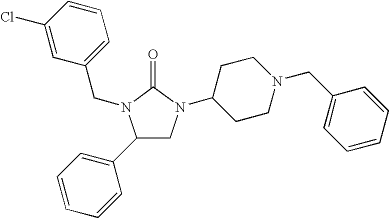 | MS (ESI POS): 460.04 (MH+)1H NMR (CDCl3 + D2O + Na2CO3):7.41-7.12 (m, 12 H); 7.07 (s, 1 H); 6.98 (m,1 H); 4.79 (d, 1 H); 4.29 (dd, 1 H); 3.88 (m,1 H); 3.61 (dd, 1 H); 3.58 (d, 1 H); 3.49 (s,2 H); 3.16 (dd, 1 H); 2.94 (m, 2 H);2.11 (ddd, 2 H); 1.79-1.61 (m, 4 H). |
| (44a) | (30d) | 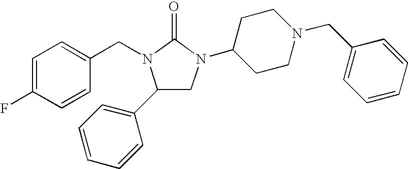 | MS (ESI POS): 444.06 (MH+)1H NMR (CDCl3 + D2O + Na2CO3)7.39-7.17 (m, 10 H); 7.06 (dd, 2 H);6.93 (dd, 2 H); 4.81 (d, 1 H); 4.25 (dd,1 H); 3.87 (m, 1 H); 3.58 (dd, 1 H);3.56 (d, 1 H); 3.49 (s, 2 H); 3.13 (dd,1 H); 2.93 (m, 2 H); 2.11 (ddd, 2 H);1.75-1.62 (m, 4 H) |
| (44a) | (30e) | 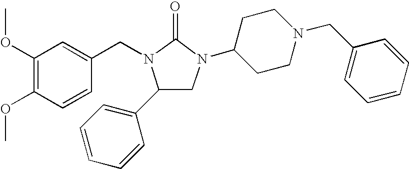 | MS (ESI POS): 486.09 (MH+)1H NMR (CDCl3 + D2O + Na2CO3):7.40-7.18 (m, 10 H); 6.75 (d, 1 H);6.64 (d, 1 H); 6.60 (dd, 1 H); 4.82 (d,1 H); 4.26 (dd, 1 H); 3.88 (m, 1 H);3.85 (s, 3 H); 3.78 (s, 3 H); 3.56 (dd,1 H); 3.49 (d, 1 H); 3.49 (s, 2 H);3.10 (dd, 1 H); 2.92 (m, 2 H); 2.11 (m,2 H); 1 .69 (m, 4 H). |
| (44a) | (30f) | 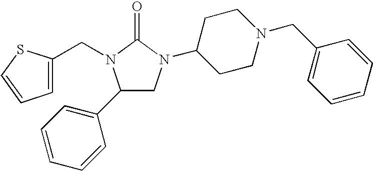 | MS (ESI POS): 432.03 (MH+)1H NMR (CDCl3): 7.42-7.16 (m,11 H); 6.90 (dd, 1 H); 6.76 (d, 1 H);4.97 (d, 1 H); 4.37 (dd, 1 H); 3.86 (m,1 H); 3.78 (d, 1 H); 3.56 (dd, 1 H);3.49 (s, 2 H); 3.12 (dd, 1 H); 2.92 (m,2 H); 2.10 (m, 2 H); 1.75-1.60 (m, 4 H). |
| (44e) | (30g) | 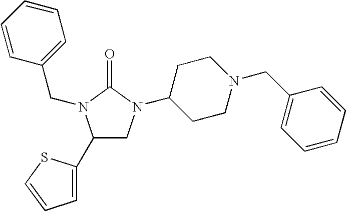 | MS (ESI POS): 432.10 (MH+)1H NMR (CDCl3): 7.33-7.20 (m,9 H); 7.19-7.13 (m, 2 H); 6.94 (dd,1 H); 6.90 (dd, 1 H); 4.88 (d, 1 H);4.59 (dd, 1 H); 3.89 (m, 1 H); 3.64 (d,1 H); 3.60 (dd, 1 H); 3.50 (s, 2 H);3.26 (dd, 1 H); 2.95 (m, 2 H); 2.11 (m,2 H); 1.81-1.64 (m, 4 H). |
| (44d) | (30h) | 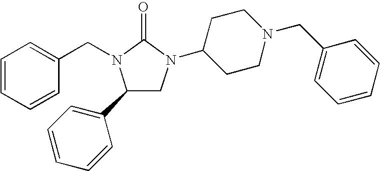 | MS (ESI POS): 425.9 (MH+)1H NMR (CDCl3 + D2O +Na2CO3): 7.40-7.17 (m, 13 H);7.09 (m, 2 H); 4.89 (d, 1 H); 4.28 (dd,1 H); 3.89 (m, 1 H); 3.58 (dd, 1 H);3.55 (d, 1 H); 3.49 (s, 2 H); 3.13 (d,1 H); 2.93 (m, 2 H); 2.11 (ddd, 2 H);1.77-1.58 (m, 4 H). |
| (44c) | (30i) | 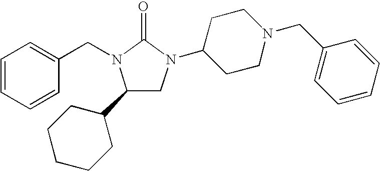 | MS (ESI POS): 432.2 (MH+)1H NMR (CDCl3): 7.63-7.38 (m,6 H); 7.37-7.15 (m, 4 H); 4.79 (d, 1 H);4.16 (s, 2 H); 4.03 (m, 1 H); 3.95 (d,1 H); 3.59 (m, 2 H); 3.34 (m, 1 H);3.16 (dd, 1 H); 3.07 (dd, 1 H); 2.75 (m,2 H); 2.27 (m, 2 H); 1.92-1.54 (m,6 H); 1.40 (m, 2 H); 1.28-1.02 (m,3 H); 0.89 (m, 2 H). |
| (44b) | (30j) | 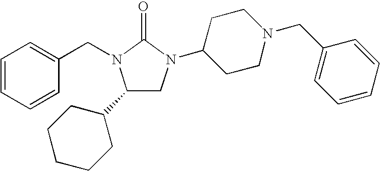 | MS (ESI POS): 432.2 (MH+)1H NMR (CDCl3 328 K): 7.67-7.30 (m, 10 H); 5.05 (s, 2 H); 4.53 (s,2 H); 3.71 (m, 2 H); 3.56 (m, 2 H);3.48-3.19 (m, 4 H); 3.68 (m, 2 H);1.98 (m, 2 H); 1.84-1.59 (m, 5 H);1.46-1.11 (m, 4 H); 0.94 (m, 2 H). |

### Procedure 16

### Final Products (30) Described in Scheme 9

### 1-(1-Benzyl-piperidin-4-yl)-3,4-diphenyl-imidazolidin-2-one (30aa)

Compound (44a) (100 mg, 0.3 mmoles) is dissolved in DMF (1 mL). Iodobenzene (0.067 mL), copper(I) iodide (60 mg) and K₂CO₃ (40 mg) are added and the resulting mixture is heated to 150° C. under nitrogen atmosphere for 4 hours. The mixture is then diluted with ethyl acetate, filtered over a celite pad and the resulting solution is dried in vacuo. The product is purified by preparative HPLC to yield 45 mg of pure product as a white solid.

Compounds described in Table 16 were synthesized following the same Procedure 18, starting from the described intermediates.

TABLE 16

| Starting | | | |
|---|---|---|---|
| material | Product | Structure | analytical |
| (44a) | (30aa) | 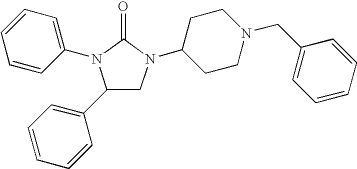 | MS (ESI POS): 412.11 (MH+)(CDCl3 + Na2CO3 + D2O):7.40 (dd, 2 H); 7.36-7.12 (m, 12 H);6.93 (tt, 1 H); 5.15 (dd, 1 H); 3.93 (m,1 H); 3.86 (dd, 1 H); 3.50 (s, 2 H);3.20 (dd, 1 H); 2.93 (m, 2 H); 2.11 (m,2 H); 1.81-1.52 (m, 4 H). |
| (17a) | (30ac) | 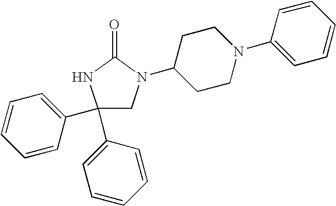 | MS (ESI POS): 398.11 (MH+)1H NMR(CDCl3 + Na2CO3 + D2O): 7.39-7.19 (m, 12 H); 6.93 (d, 2 H); 6.84(dd, 1 H); 4.02 (m, 1 H); 3.96 (s, 2 H);3.74 (m, 2 H); 2.85 (m, 2 H); 1.91-1.78 (m, 4 H). |

### Procedure 17

### Final Products (18) Described in Scheme 5

Tributyltin Azide

Sodium azide (265 mg, 4 mmoles) is dissolved in water (4 mL). The solution is cooled to 0° C. and tributyltin chloride (1.08 mL, 4 mmoles) is added dropwise. The solution is stirred at room temperature for 2 hours; then the aqueous mixture is extracted twice with methylene chloride, the organic phase is dried over MgSO₄ and the solvent is evaporated in vacuo, to yield 980 mg of tributyltin azide.

### 4,4-Diphenyl-1-[1-(1H-tetrazol-5-ylmethyl)-piperidin-4-yl]-imidazolidin-2-one

Compound (18 m) (100 mg, 0.27 mmoles) is reacted with neat tributyltin azide (980 mg) at the temperature of 100° C. for 4 hours. The reaction mixture is then cooled at room temperature, methanol (3 mL) and 2N HCl (1 mL) are added and the resulting mixture is stirred at room temperature for 1 hour. The solution is extracted with ethyl acetate; the organic phase is washed with brine, dried over anhydrous sodium sulphate and concentrated under reduced pressure to afford an oil that is purified by chromatography on silica gel (eluent: dichloromethane:methanol 95:5) to yield the desired product as a yellow solid (61 mg).

Compounds described in Table 17 were synthesized following the same procedure, starting from the corresponding cyano derivatives.

TABLE 17

| Starting | | | |
|---|---|---|---|
| material | Product | Structure | analytical |
| (18r′) | (18ao) | 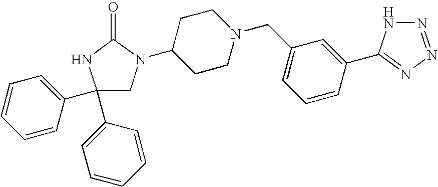 | MS (ESI POS): 479.9 (MH+)′1H NMR (DMSO 368 K): 7.99 (sbr, 1 H); 7.91 (dd, 1 H); 7.49-7.28 (m, 11 H); 7.22 (dd, 2 H);3.94 (s, 2 H); 3.85 (m, 2 H); 3.60 (s,2 H); 2.55 (m, 2 H); 2.15 (m, 2 H),1.76 (m, 2 H); 1.58 (m, 2 H). |
| (18m) | (18ap) | 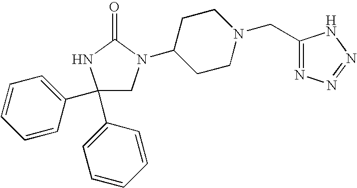 | MS (ESI POS): 404.1 (MH+)1H NMR (DMSO): 8.04 (s br,1 H); 7.39-7.19 (m, 10 H); 5.74 (s,1 H); 4.28 (s br, 2 H); 3.89 (s, 2 H);3.44-3.26 (m, 3 H); 2.73 (m, 2 H);1.83 (m, 2 H); 1.66 (m, 2 H). |

### Procedure 18

### (3-Hydroxymethyl-phenyl)-Carbamic Acid Ethyl Ester

3-amino-benzyl alcohol (1.0 g, 8.1 mmoles) (1.36 mL, 9.7 mmoles) is dissolved in dry dichloromethane (15 mL). The resulting solution is cooled to 0° C. and ethyl chloroformiate (0.86 mL, 8.9 mmoles) is added dropwise. The mixture is stirred at 0° C. for 2 hours, then aqueous K₂CO₃ (1M, 20 mL) is added and the mixture is extracted twice with dichloromethane, the organic phase is washed with brine, dried over anhydrous sodium sulphate and concentrated under reduced pressure to afford the desired product as an orange oil which is purified by chromatography on silica gel (eluent: dichloromethane:methanol 95:5) to yield the desired product as a light-yellow oil (1.0 g).

### Methanesulfonic acid 3-ethoxycarbonylamino-benzyl Ester

(3-Hydroxymethyl-phenyl)-carbamic acid ethyl ester (0.63 g, 3.2 mmoles) and triethylamine (0.59 mL, 4.2 mmoles) are dissolved in dry dichloromethane (10 mL). The resulting solution is cooled to 0° C. and mehtansulhonyl chloride (0.28 mL, 3.58 mmoles) is added dropwise. The resulting mixture is stirred at room temperature for 2 hours, then aqueous K2CO3 (1M, 20 mL) is added and the mixture is extracted twice with dichloromethane; the organic phase is washed with brine, dried over anhydrous sodium sulphate and concentrated under reduced pressure. The desired product is further purified by chromatography on silica gel (eluent: ethyl acetate:hexane 9:1), to yield a colorless oil (0.13 g).

Compound (18ag):

### 1-[1-(3-Methylamino-benzyl)-piperidin-4-yl]-4,4-diphenyl-imidazolidin-2-one

Compound (18aq) was synthesized starting from intermediate (17a) in two steps:

Step 1: Alkylation of (17a) with methanesulfonic acid 3-ethoxycarbonylamino-benzyl ester was performed as described in procedure 12, to yield {3-[4-(2-Oxo-4,4-diphenyl-imidazolidin-1-yl)-piperidin-1-ylmethyl]-phenyl}-carbamic acid ethyl ester

Step 2: Reduction of {3-[4-(2-Oxo-4,4-diphenyl-imidazolidin-1-yl)-piperidin-1-ylmethyl]-phenyl}-carbamic acid ethyl ester to the desired compound was performed as described in procedure 6.

TABLE 18

| Starting | | | |
|---|---|---|---|
| material | Product | Structure | analytical |
| (17a) | (18aq) | 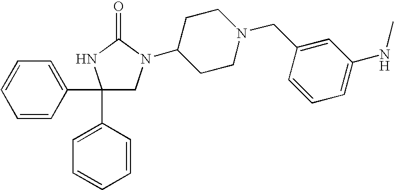 | MS (ESI POS): 441.11 (MH+)′1H NMR (CDCl3 + Na2CO3 + D2O):7.38-7.19 (m, 10 H); 7.11 (dd, 1 H); 6.64(d, 1 H); 6.57 (d, 1 H); 6.50 (dd, 1 H);3.95 (s, 2 H); 3.85 (m, 1 H); 3.42 (s, 2 H);2.95 (m, 2 H); 2.82 (s, 3 H); 2.07 (m, 2 H);1.78-1.67 (m, 4 H). |

The compounds of the present invention display antimuscarinic M₃ activity in a radioligand binding assay following the methods previously described. Binding affinities of the compounds of the invention versus M₃ receptor range from 0.1 to 2000 nM (Ki); most preferred compounds have Ki ranging from 0.1 to 100 nM. The following data illustrate some examples:

| compound | M₃ (Ki, nM) | M₂ (Ki, nM) |
|---|---|---|
| 27c | 2.5 | 73.3 |
| 27a | 1.9 | 80.1 |
| 27h | 2.77 | 206 |
| 27i | 2.7 | 96.8 |
| 18e | 3.1 | 327 |
| 18i | 2.37 | 224 |
| 18ab | 6.7 | 955 |
| 18ad | 2.75 | 187 |
| 18ag | 1.72 | 106.8 |
| 18an | 4.83 | 1141 |
| 19b1 | 0.67 | 5.4 |
| 19bq | 1.62 | 88.5 |
| 19bp | 1.96 | 80.9 |
| 19bt | 1.84 | 94.5 |
| 19bv | 0.75 | 21.9 |
| 19by | 3.01 | 108.1 |
| 19bz | 2.61 | 268 |

1. A compound of general formula (I) 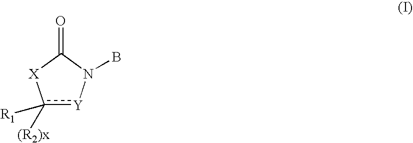
wherein:
R₁, represents
linear or branched C₁-C₇ alkyl;
C₃-C₇ cycloalkyl;
phenyl,
benzyl,
phenyloxymethyl, or
a single or fused heterocycle, optionally substituted with one or more of the following groups: F, Cl, Br, linear or branched C₁-C₆ alkyl, C₃-C₆ cycloalkyl, methylendioxy, ethylendioxy, vinyl, CF₃, NO₂, CN, COOH, OCF₃, CH₂OR₄, OR₄, NR₄R₅, SO₂NR₄R₅, CONR₄R₅, SR₄, SO₂R₄, COR₄, wherein R₄ is H, linear or branched C₁-C₆ alkyl, phenyl, benzyl or a single or fused heterocycle optionally substituted with F, Cl, Br, linear or branched C₁-C₆ alkyl, C₃-C₆ cycloalkyl, methylendioxy, ethylendioxy, vinyl, CF₃, NO₂, CN, CH₂OH and R₅ is H, linear or branched C₁-C₇ alkyl, CO-(linear or branched C₁-C₇ alkyl) or R₄ and R₅ can form a single or fused heterocycle comprising up to 8 atoms;
R₂ is H or has the same meanings as R₁
Y represents:
C═O;
CHOH;
(CH₂)ₘ, wherein m is an integer from 1 to 3; or a
X represents:
sulfur or a
NR₇ group, wherein R₇ is hydrogen or a G-R₆ group, in which G is selected from CO, SO₂, (CH₂)ₙ, (CH₂)ₙCONH with n=0-3 and R₆ is H, a COOH group or has the same meanings as R₁; B is selected from one of the following groups:
a1) 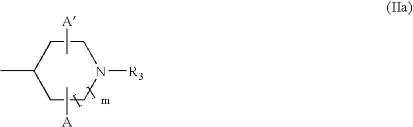
wherein A and A′ represent, independently from one another, hydrogen, linear or branched C₁-C₄ alkyl groups,
m is 0-2 and
R₃ is a M-R₆ group, wherein M is selected from CO, CONH, SO₂, (CH₂)ₙ, (CH₂)ₙCONH with n=1-3 and R₆ is H, a COOH group or has the same meanings as R₁;
a2) 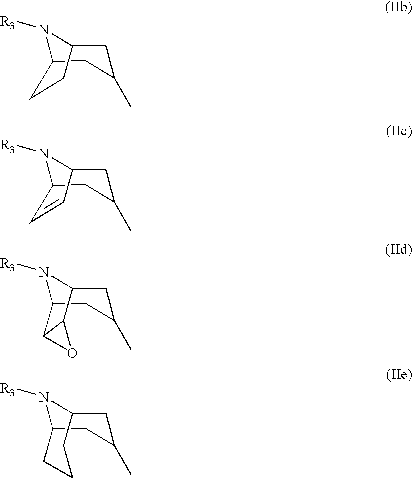
R₃ is a M-R₆ group, wherein M is selected from CO, CONH, SO₂, (CH₂)ₙ, (CH₂)ₙCONH with n=0-3 and R₆ is H, a COOH group or has the same meanings as R₁;
b) 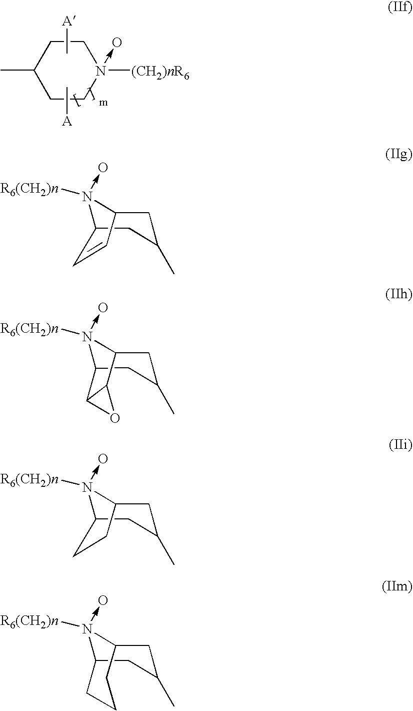
wherein R₆, m and n are as defined above;
c) 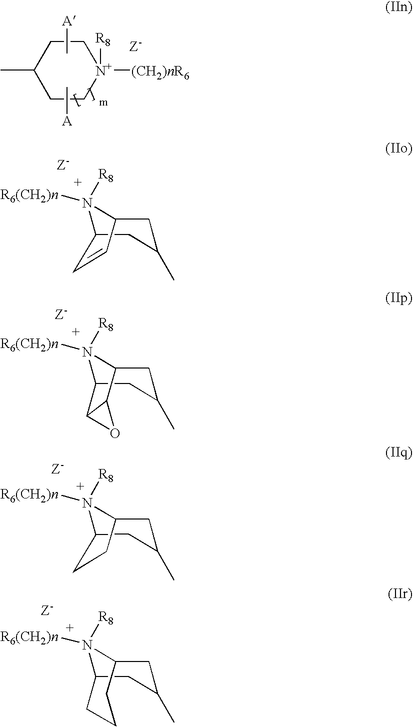
wherein:
R₆, m and n are as defined above and R₈ has the same meanings as R₁, or (CH₂)ₙR₆ and R₈, together with the nitrogen atom they are bound to, form a 4 to 7-membered heterocyclic ring, optionally substituted by a phenyl ring or optionally fused with a benzene ring or a heterocycle as defined above;
Z⁻ is a pharmaceutically acceptable anion; and wherein single or fused heterocycle is a heterocyclic ring containing from 5 to 10 ring atoms, and comprising up to 4 heteroatoms selected from S, N, O in each ring, selected from pyrrole, pyrazole, furan, thiophene, indole, benzofuran, benzothiophene, imidazole, oxazole, isoxazole, thiazole, benzimidazole, benzoxazole, benzothiazole, pyridine, pyrimidine, pyrazine, quinoline, isoquinoline, quinazoline, and all the corresponding saturated and partially saturated heterocycles.

2. A compound according to claim 1 wherein Y represents C═O.

3. A compound according to claim 1 wherein Y represents (CH₂)ₘ, and m=1.

4. A compound according to claim 2 wherein, X represents a NR₇ group.

5. A compound according to claim 2 wherein:
R₁ is selected from the group consisting of optionally substituted phenyl, cyclopentyl, cyclohexyl, benzyl, 2-thienyl;
R₂ is selected from the group consisting of hydrogen, optionally substituted phenyl or phenoxymethyl, cyclopentyl, cyclohexyl; 2-thienyl, methyl and
R₇ is hydrogen or a G-R₆ group wherein G is (CH₂)ₙ with n=1 and R₆ is hydrogen, methyl, substituted or unsubstituted phenyl or single or fused heterocycle.

6. A compound according to claim 5 wherein R₁ and R₂ are both phenyl.

7. A compound according to claim 1 wherein B is a group of formula (IIa) 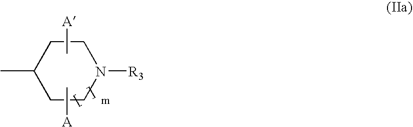
wherein A and A′ are hydrogen with m=0-2 and R₃ is a M-R₆ group, wherein M is (CH₂)ₙ with n=1-3 and R₆ is hydrogen, substituted or unsubstituted phenyl, phenoxy, cyclohexyl or single or fused heterocycle.

8. A compound according to claim 1 wherein B is a group of formula (IIb) 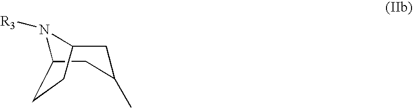
a group of formula (IId) 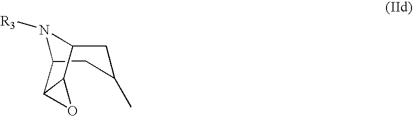
or a group of formula (IIf) 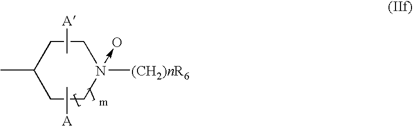
wherein A and A′ are hydrogen with m=0-2 and R₃ is a M-R₆ group, wherein M is (CH₂)ₙ with n=0-3 and R₆ is hydrogen, substituted or unsubstituted phenyl, phenoxy, cyclohexyl or single or fused heterocycle.

9. A compound according to claim 7, represented by the following formula

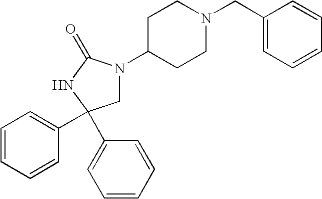

10. A compound according to claim 1 wherein B is a group of formula (IIn) 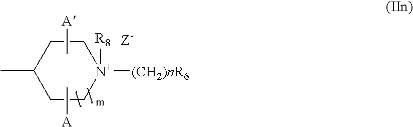
or a group of formula (IIq) 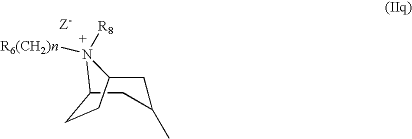
or a group of formula (IIp) 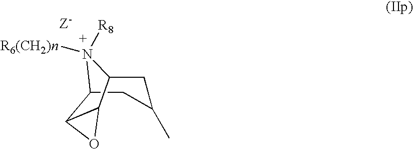
wherein A and A′ are hydrogen,
m is 0-2,
n is 1-3,
R₆ is hydrogen, phenyl, single or fused heterocycle, C₁-C₄ alkyl optionally substituted by SR₄, SO₂R₄, CN, OR₄, COR₄, CONHR₄, wherein R₄ is selected from optionally substituted phenyl, benzyl, 2- or 3-thienyl, 2-, 3- or 4-pyridinyl, C₁-C₄ alkyl and R₈ is selected from: methyl, 2-thienyl-propyl, cyclohexylmethyl, optionally substituted benzyl, phenoxyethyl, 2-N,N-dimethylaminoethyl; 2-tetrahydroturylmethyl, carboxymethyl, 2-(5-aminopiridinylmethyl), 2-(SO₃H)-ethyl or (CH₂)ₙR₆ and R₈, together with the nitrogen atom they are bound to, form one of the following groups: 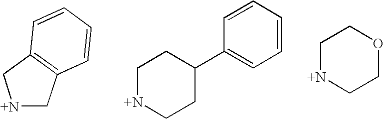
and Z is a pharmaceutically acceptable anion.

11. A compound according to claim 19 and represented by the following formula: 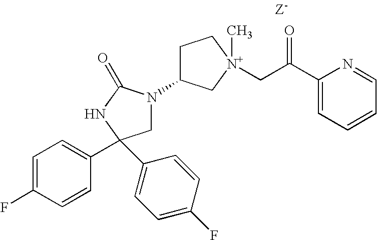
wherein Z⁻ is a pharmaceutically acceptable anion.

12. A compound according to, claim 1 wherein Z⁻ is a pharmaceutically acceptable anion selected from chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

13. A process for the preparation of a compound of general formula (I) which comprises the steps of:
(a) functionalizing intermediates of formula (a′) at the nitrogen atom in position 3 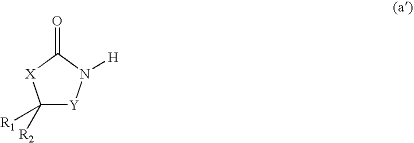
by reaction with an amino-alcohol suitably protected at the amino group with a protecting group (PG) selected from tert-butyloxycarbonyl (BOC), benzyloxycarbonyl (Cbz), benzyl or methyl derivative, or by deprotonation of the nitrogen at position 3 and subsequent reaction with a mesylate derivative of an amino-alcohol suitably protected at the amino group with a protecting group (PG) or with a similar derivative in which the alcohol group has been activated as leaving group to give compounds of formula (a″) 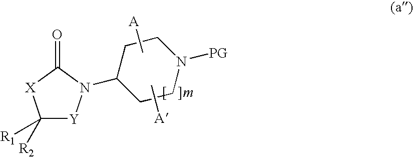
(b) removing the protecting group PG by methods described in the literature to obtain final compounds or intermediates that can be further functionalized by the introduction of a residue R₃ on the secondary amino group by methods described in the literature to give compounds of formula (I) wherein B is selected from one of the following groups:
a1) 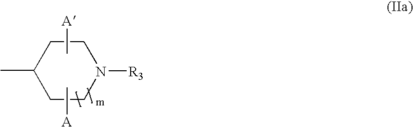
wherein A and A′ represent, independently from one another, hydrogen, linear or branched C₁-C₄ alkyl groups,
m is 0-2 and
R₃ is a M-R₆ group, wherein M is selected from CO, CONH, SO₂, (CH₂)ₙ, (CH₂)ₙCONH with n=1-3 and R₆ is H, a COOH group or has the same meanings as R₁;
a2) 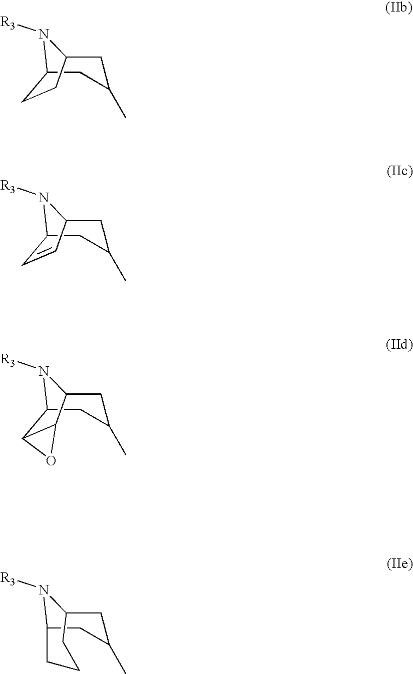
wherein A and A′ represent, independently from one another, hydrogen, linear or branched C₁-C₄ alkyl groups,
m=0-2 and
R₃ is a M-R₆ group, wherein M is selected from CO, CONH, SO₂, (CH₂)ₙ, (CH₂)ₙCONH with n=0-3 and R₆ is H, a COOH group or has the same meanings as R₁; and optionally
(c) further functionalizing said compounds on the same nitrogen atom,
by treatment with an organic or inorganic acid selected among hydrochloric acid, hydrobromic acid, oxalic acid, fumaric acid, tartaric acid to give ammonium salts;
by treatment with m-chloroperbenzoic acid or oxone to be transformed in N-oxide derivatives of formula (I) wherein B is selected from one of the following groups 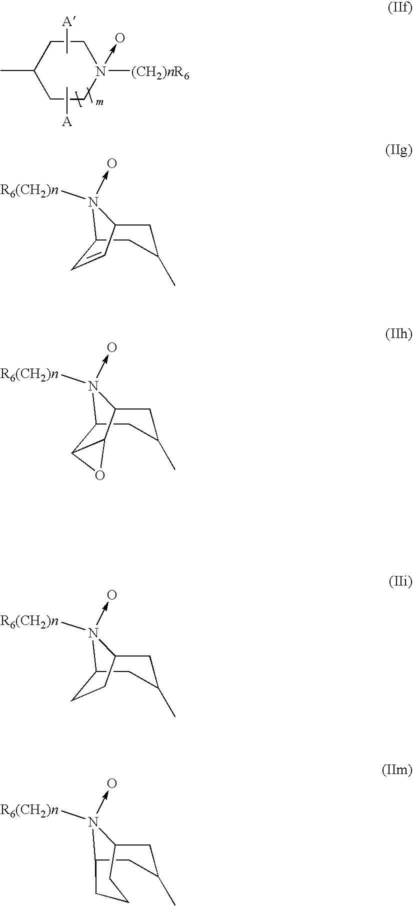
wherein R₆, m and n are as defined above;
by treatment with suitable alkylating agents to be transformed in quaternary ammonium salts of formula (I) wherein B is selected from one of the following groups 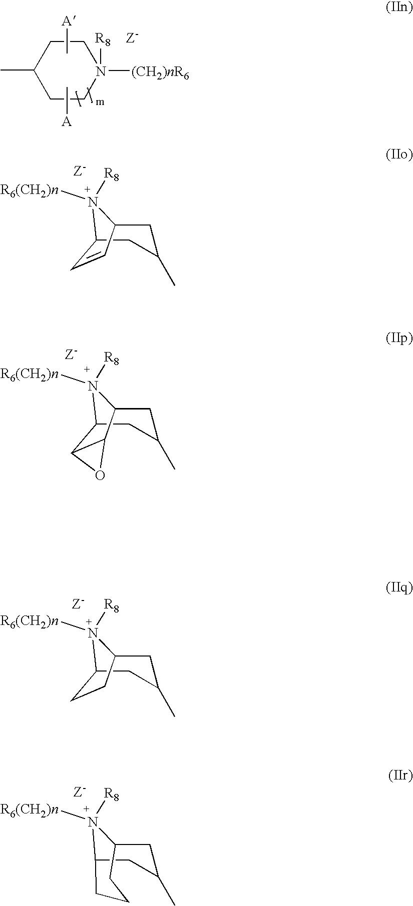
wherein:
R₆, m and n are as defined above and R₈ has the same meanings as R₁, or (CH₂)ₙR₆ and R₈, together with the nitrogen atom they are bound to, form a 4 to 7-membered heterocyclic ring, optionally substituted by a phenyl ring or optionally fused with a benzene ring or a heterocycle as defined above; and
Z⁻ is a pharmaceutically acceptable anion.

14. A method of treating a respiratory, urinary or gastrointestinal disease, urinary incontinence bladder-related diseases; or irritable bowel syndrome which comprises administering to a subject in need thereof a therapeutically effective amount of a compound as claimed in claim 1.

15. The method according to claim 14 wherein the respiratory disease is asthma, chronic obstructive pulmonary disease (COPD), chronic bronchitis, cough, emphysema or rhinitis.

16. A pharmaceutical composition containing a compound of claim 1 in admixture with suitable carriers and/or excipients.

17. A compound according to claim 3, wherein X represents a NR₇ group.

18. A compound according to claim 3 wherein:
R₁ is selected from the group consisting of optionally substituted phenyl, cyclopentyl, cyclohexyl, benzyl, 2-thienyl;
R₂ is selected from the group consisting of hydrogen, optionally substituted phenyl or phenoxymethyl, cyclopentyl, cyclohexyl; 2-thienyl, methyl and
R₇ is hydrogen or a G-R₆ group wherein G is (CH₂)ₙ with n=1 and R₆ is hydrogen, methyl, substituted or unsubstituted phenyl or single or fused heterocycle.

19. A compound according to claim 2 wherein B is a group of formula (IIa) 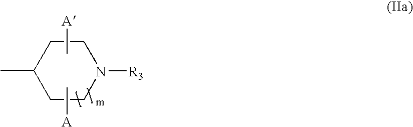
wherein A and A′ are hydrogen with m=0-2 and R₃ is a M-R₆ group, wherein M is (CH₂)ₙ with n=1-3 and R₆ is hydrogen, substituted or unsubstituted phenyl, phenoxy, cyclohexyl or single or fused heterocycle.

20. A compound according claim 2 wherein B is a group of formula (IIb) 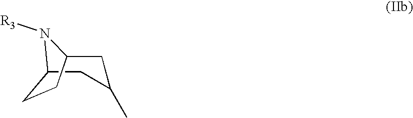
a group of formula (IId) 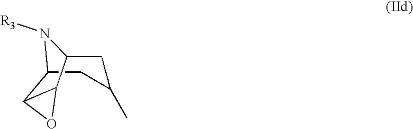
or a group of formula (IIf) 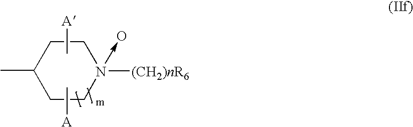
wherein A and A′ are hydrogen with m=0-2 and R₃ is a M-R₆ group, wherein M is (CH₂) n with n=0-3 and R6 is hydrogen, substituted or unsubstituted phenyl, phenoxy, cyclohexyl or single or fused heterocycle.
